SEQUENCE LISTING (102 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1260 aa, Severe acute respiratory syndrome coronavirus 2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1260 aa, Severe acute respiratory syndrome coronavirus 2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 201 aa, Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 6 - protein, 1268 aa, Severe acute respiratory syndrome coronavirus 2
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1255 aa, Severe acute respiratory syndrome coronavirus 2
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1268 aa, Severe acute respiratory syndrome coronavirus 2
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1255 aa, Severe acute respiratory syndrome coronavirus 2
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 201 aa, Severe acute respiratory syndrome coronavirus 2
NITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKST

SEQ ID NO: 11 - protein, 201 aa, synthetic construct
NITNLCPFGEVFNATRFASVYAWNRKRISNCVADYSVLYNSASFSTFKCYGVSPTELNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEDFNCYFPLQPYGFQPTNGVGYQPYRVVVLSFELLHAPATVCGPKKST

SEQ ID NO: 12 - protein, 201 aa, synthetic construct
NITNLCPFGEVFNATRFASVYAWNRKRISNCVADYSVLYNSASFSDFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGPNCYFPLQPYGFQPTNGVGYQPYRVVVLSFELLHAPATVCGPKKST

SEQ ID NO: 13 - protein, 201 aa, synthetic construct
NITNLCPFGEVFNATRFASVYAWNRKRISNCVADYSVLYNSASFSTFKCYGVSPTELNDLCFTNVYADSFVIRGDEVRQIAPGQTGKIADYNYKLPDDFTGCVIAWNSNNLDSKVGGNYNYDYRLFRKSNLKPFERDISTEIYQAGSTPCNGVEGFNCYFPLQSYGFQPTNGVGYQPYRVVVLMFELLHAPATVCGPKKST

SEQ ID NO: 14 - protein, 201 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGPNCYFPLQSYGFQPTNGVGYQPYRVVVLSFELLHAPATVCGPKKST

SEQ ID NO: 15 - protein, 201 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQPYGFQPTNGVGYQPYRVVVLSFELLHAPATVCGPKKST

SEQ ID NO: 16 - protein, 201 aa, synthetic construct
NITNLCPFGEVFNATRFASVYAWNRKRISNCVADYSVLYNSASFSDFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 17 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTELNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEDFNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 18 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSDFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGPNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 19 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTELNDLCFTNVYADSFVIRGDEVRQIAPGQTGKIADYNYKLPDDFTGCVIAWNSNNLDSKVGGNYNYDYRLFRKSNLKPFERDISTEIYQAGSTPCNGVEGFNCYFPLQSYGFQPTNGVGYQPYRVVVLMFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGPNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSDFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTELNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEDFNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 24 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSDFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGPNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTELNDLCFTNVYADSFVIRGDEVRQIAPGQTGKIADYNYKLPDDFTGCVIAWNSNNLDSKVGGNYNYDYRLFRKSNLKPFERDISTEIYQAGSTPCNGVEGFNCYFPLQSYGFQPTNGVGYQPYRVVVLMFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 26 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGPNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 27 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 28 - protein, 1260 aa, synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSDFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 29 - protein, 201 aa, synthetic construct
NITNLCPFHEVFNATTFASVYAWNRTRISNCVADYSVLYNFAPFFAFKCYGVSPTKLNDLCFTNVYADSFVIRGNEVSQIAPGQTGTIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKST

SEQ ID NO: 30 - protein, 201 aa, synthetic construct
NITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSNLKPFERDISTEIYQAGNKPCNGVAGVNCYFPLQSYGFRPTYGVGHQPYRVVVLSFELLHAPATVCGPKKST

SEQ ID NO: 31 - protein, 201 aa, synthetic construct
NITNLCPFDEVFNATRFASVYAWNRKRISNCVADYSVLYNFAPFFAFKCYGVSPTKLNDLCFTNVYADSFVIRGNEVSQIAPGQTGYIADYNYKLPDDFTGCVIAWNSNKLDSEVGGNYNYRYRLFRKSNLKPFERDISTEIYQAGNKPCNGVAGVNCYFPLQSYGFRPTYGVGHQPYRVVVLSFELLHAPATVCGPKKST

SEQ ID NO: 32 - protein, 201 aa, synthetic construct
NITNLCPFDEVFNATRFASVYAWNRKRISNCVADYSVLYNFAPFFAFKCYGVSPTKLNDLCFTNVYADSFVIRGNEVSQIAPGQTGHIADYNYKLPDDFTGCVIAWNSNKLDSNVGGNYNYRYRLFRKSNLKPFERDISTEIYQAGNKPCNGVAGVNCYFPLQSYGFRPTYGVGHQPYRVVVLSFELLHAPATVCGPKKST

SEQ ID NO: 33 - protein, 201 aa, synthetic construct
NITNLCPFDEVFNATRFASVYAWNRKRISNCVADYSVLYNFAPFFAFKCYGVSPTKLNDLCFTNVYADSFVIRGNEVSQIAPGQTGIIADYNYKLPDDFTGCVIAWNSNKLDSMVGGNYNYRYRLFRKSNLKPFERDISTEIYQAGNKPCNGVAGVNCYFPLQSYGFRPTYGVGHQPYRVVVLSFELLHAPATVCGPKKST

SEQ ID NO: 34 - protein, 201 aa, synthetic construct
NITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSQVGGNYNYRYRLFRKSNLKPFERDISTEIYQAGNKPCNGVAGVNCYFPLQSYGFRPTYGVGHQPYRVVVLSFELLHAPATVCGPKKST

SEQ ID NO: 35 - protein, 201 aa, synthetic construct
NITNLCPFDEVFNATSFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKDGGNYNYRYRLFRKSNLKPFERDISTEIYQAGNKPCNGVAGVNCYFPLQSYGFRPTYGVGHQPYRVVVLSFELLHAPATVCGPKKST

SEQ ID NO: 36 - protein, 201 aa, synthetic construct
NITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKGGGNYTYRYRLFRKSNLKPFERDISTEIYQAGNKPCNGVAGVNCYFPLQSYGFRPTYGVGHQPYRVVVLSFELLHAPATVCGPKKST

SEQ ID NO: 37 - protein, 201 aa, synthetic construct
NITNLCPFHEVFNATTFASVYAWNRTRISNCVADYSVLYNFAPFFAFKCYGVSPTKLNDLCFTNVTADSFVIRGNEVSQIAPGQTGTIADYNYKLPDDFTGCVIAWNSNKLDSTVGGNYNYRYRLFRKSKLKPFERDNSTEIYQAGNKPCNGVAGVNCYFPLQSYGFRPTYGVGHQPYRVVVLSFELLHAPATVCGPKKST

SEQ ID NO: 38 - protein, 201 aa, synthetic construct
NITNLCPFHEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKVSGNYNYRYRLFRKSNLKPFERDNSTEIYQAGNKPCNGVAGVNCYFPLQSYGFRPTYGVGHQPYRVVVLSFELLHAPATVCGPKKST

SEQ ID NO: 39 - protein, 201 aa, synthetic construct
NITNLCPFDEVFNATRFASVYAWNRKRISNCVADYSVLYNFAPFFAFKCYGVSPTKLNDLCFTNVTADSFVIRGNEVSQIAPGQTGYIADYNYKLPDDFTGCVIAWNSNKLDSEVGGNYNYRYRLFRKSNLKPFERDNSTEIYQAGNKPCNGVAGVNCYFPLQSYGFRPTYGVGHQPYRVVVLSFELLHAPATVCGPKKST

SEQ ID NO: 40 - protein, 201 aa, synthetic construct
NITNLCPFDEVFNATRFASVYAWNRKRISNCVADYSVLYNFAPFFAFKCYGVSPTKLNDLCFTNVTADSFVIRGNEVSQIAPGQTGHIADYNYKLPDDFTGCVIAWNSNKLDSNVGGNYNYRYRLFRKSNLKPFERDNSTEIYQAGNKPCNGVAGVNCYFPLQSYGFRPTYGVGHQPYRVVVLSFELLHAPATVCGPKKST

SEQ ID NO: 41 - protein, 201 aa, synthetic construct
NITNLCPFDEVFNATRFASVYAWNRKRISNCVADYSVLYNFAPFFAFKCYGVSPTKLNDLCFTNVTADSFVIRGNEVSQIAPGQTGIIADYNYKLPDDFTGCVIAWNSNKLDSMVGGNYNYRYRLFRKSNLKPFERDNSTEIYQAGNKPCNGVAGVNCYFPLQSYGFRPTYGVGHQPYRVVVLSFELLHAPATVCGPKKST

SEQ ID NO: 42 - protein, 201 aa, synthetic construct
NITNLCPFDEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSQVGGNYNYRYRLFRKSNLKPFERDNSTEIYQAGNKPCNGVAGVNCYFPLQSYGFRPTYGVGHQPYRVVVLSFELLHAPATVCGPKKST

SEQ ID NO: 43 - protein, 201 aa, synthetic construct
NITNLCPFDEVFNATSFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKDGGNYNYRYRLFRKSNLKPFERDNSTEIYQAGNKPCNGVAGVNCYFPLQSYGFRPTYGVGHQPYRVVVLSFELLHAPATVCGPKKST

SEQ ID NO: 44 - protein, 201 aa, synthetic construct
NITNLCPFDEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKGGGNYTYRYRLFRKSNLKPFERDNSTEIYQAGNKPCNGVAGVNCYFPLQSYGFRPTYGVGHQPYRVVVLSFELLHAPATVCGPKKST

SEQ ID NO: 45 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRTRISNCVADYSVLYNFAPFFAFKCYGVSPTKLNDLCFTNVYADSFVIRGNEVSQIAPGQTGTIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 46 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 47 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGYIADYNYKLPDDFTGCVIAWNSNKLDSEVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 48 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGHIADYNYKLPDDFTGCVIAWNSNKLDSNVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 49 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGIIADYNYKLPDDFTGCVIAWNSNKLDSMVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 50 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSQVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 51 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATSFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKDGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 52 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKGGGNYTYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 53 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRTRISNCVADYSVLYNFAPFFAFKCYGVSPTKLNDLCFTNVTADSFVIRGNEVSQIAPGQTGTIADYNYKLPDDFTGCVIAWNSNKLDSTVGGNYNYRYRLFRKSKLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 54 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKVSGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 55 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGYIADYNYKLPDDFTGCVIAWNSNKLDSEVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 56 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGHIADYNYKLPDDFTGCVIAWNSNKLDSNVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 57 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGIIADYNYKLPDDFTGCVIAWNSNKLDSMVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 58 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSQVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 59 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATSFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKDGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 60 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKGGGNYTYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 61 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRTRISNCVADYSVLYNFAPFFAFKCYGVSPTKLNDLCFTNVYADSFVIRGNEVSQIAPGQTGTIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 62 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 63 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGYIADYNYKLPDDFTGCVIAWNSNKLDSEVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 64 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGHIADYNYKLPDDFTGCVIAWNSNKLDSNVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 65 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGIIADYNYKLPDDFTGCVIAWNSNKLDSMVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 66 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSQVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 67 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATSFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKDGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 68 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKGGGNYTYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 69 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRTRISNCVADYSVLYNFAPFFAFKCYGVSPTKLNDLCFTNVTADSFVIRGNEVSQIAPGQTGTIADYNYKLPDDFTGCVIAWNSNKLDSTVGGNYNYRYRLFRKSKLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 70 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKVSGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 71 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGYIADYNYKLPDDFTGCVIAWNSNKLDSEVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 72 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGHIADYNYKLPDDFTGCVIAWNSNKLDSNVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 73 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGIIADYNYKLPDDFTGCVIAWNSNKLDSMVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 74 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSQVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 75 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATSFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKDGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 76 - protein, 1255 aa, synthetic construct
QCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKGGGNYTYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 77 - protein, 805 aa, Homo sapiens [sequence omitted]

SEQ ID NO: 78 - protein, 788 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 79 - protein, 4 aa, Severe acute respiratory syndrome coronavirus 2
RRAR

SEQ ID NO: 80 - protein, 4 aa, unidentified
QQAQ

SEQ ID NO: 81 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTELNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEDFNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 82 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSDFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGPNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 83 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTELNDLCFTNVYADSFVIRGDEVRQIAPGQTGKIADYNYKLPDDFTGCVIAWNSNNLDSKVGGNYNYDYRLFRKSNLKPFERDISTEIYQAGSTPCNGVEGFNCYFPLQSYGFQPTNGVGYQPYRVVVLMFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 84 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGPNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 85 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQPYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 86 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSDFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 87 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRTRISNCVADYSVLYNFAPFFAFKCYGVSPTKLNDLCFTNVYADSFVIRGNEVSQIAPGQTGTIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 88 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 89 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGYIADYNYKLPDDFTGCVIAWNSNKLDSEVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 90 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGHIADYNYKLPDDFTGCVIAWNSNKLDSNVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 91 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGIIADYNYKLPDDFTGCVIAWNSNKLDSMVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 92 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSQVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 93 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATSFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKDGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 94 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKGGGNYTYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 95 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRTRISNCVADYSVLYNFAPFFAFKCYGVSPTKLNDLCFTNVTADSFVIRGNEVSQIAPGQTGTIADYNYKLPDDFTGCVIAWNSNKLDSTVGGNYNYRYRLFRKSKLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 96 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKVSGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 97 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGYIADYNYKLPDDFTGCVIAWNSNKLDSEVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 98 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGHIADYNYKLPDDFTGCVIAWNSNKLDSNVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 99 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGIIADYNYKLPDDFTGCVIAWNSNKLDSMVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 100 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSQVGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 101 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATSFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKDGGNYNYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 102 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVTADSFVIRGNEVSQIAPGQTGNIADYNYKLPDDFTGCVIAWNSNKLDSKGGGNYTYRYRLFRKSNLKPFERDNSTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2025/0333452 A1
(43) Pub. Date: Oct. 30, 2025

(54) VACCINES AND RELATED METHODS

(71) Applicant: Flagship Pioneering Innovations VII, LLC, Cambridge, MA (US)

(72) Inventors: Ellen Lovisa Larsdotter AFZELIUS, Belmont, MA (US); Daniel William Menon ACKER, Arlington, MA (US); Alexander Patrick GOLDSMITH, Winchester, MA (US); Patrick Leo JONES, Boston, MA (US); Adam Spier DINGENS, Cambridge, MA (US); Yvonne Hoi Yan Chan, Cambridge, MA (US); Diego Souto Maior MOURAO SA, Reading, MA (US)

(21) Appl. No.: 19/257,614

(22) Filed: Jul. 2, 2025

Related U.S. Application Data
(63) Continuation of application No. PCT/US24/10810, filed on Jan. 9, 2024.
(60) Provisional application No. 63/479,073, filed on Jan. 9, 2023.
(60) Provisional application No. 63/504,508, filed on May 26, 2023.

(51) Int. Cl. C07K 14/005; A61K 39/00; A61K 39/215

(52) U.S. Cl. CPC C07K 14/005; A61K 39/215; A61K 2039/53; A61K 2039/55555; C12N 2770/20034

(57) ABSTRACT

Provided herein are SARS-CoV-2 spike proteins and polypeptides (e.g., SARS-CoV-2 spike proteins and polypeptide immunogens (and immunogenic fragments and immunogenic variants thereof)) comprising at least one set of amino acid substitutions, and nucleic acid molecules encoding the same. Further provided herein are compositions (e.g., pharmaceutical compositions) and vaccines comprising the same for use in e.g., the prevention, treatment, and/or amelioration of a SARS-CoV-2 infection; vaccination against SARS-CoV-2.

### RELATED APPLICATIONS

This application claims priority to U.S. Ser. No. 63/479,073, filed Jan. 9, 2023, and U.S. Ser. No. 63/504,508, filed May 26, 2023, the entire contents of each of which is incorporated herein by reference.

### 1. FIELD

This disclosure relates to SARS-CoV-2 spike proteins (e.g., SARS-CoV-2 spike protein immunogens (and immunogenic fragments and/or immunogenic variants thereof)), that comprise at least one set of amino acid substitutions (described herein) and nucleic acid molecules encoding the same. The disclosure further relates to compositions comprising of the same (e.g., vaccine compositions, pharmaceutical compositions) and methods of making and utilizing the same.

### 2. BACKGROUND

Coronaviruses are a family of enveloped, positive-sense, single stranded RNA viruses that infect a wide variety of mammalian and avian species. The viral genome is packaged into a capsid that is comprised of the viral nucleocapsid protein and surrounded by a lipid envelope. Embedded in the lipid envelope are several proteins, including, the membrane protein, the envelope small membrane protein, hemagglutinin-esterase, and the spike protein. Human coronaviruses typically cause respiratory illnesses, and include, e.g., severe acute respiratory syndrome coronavirus 2 (SARS-CoV-2), severe acute respiratory syndrome coronavirus 1 (SARS-CoV-1), and Middle East respiratory syndrome (MERS-CoV).

SARS-Cov-2 emerged in humans in 2019, spread rapidly, and led to an ongoing global pandemic. SARS-CoV-2 is the cause of the coronavirus disease 2019 (COVID-19). COVID-19 has caused a continuing public health crisis, with millions of deaths and severe illness attributed to COVID-19 worldwide. Protection against COVID-19 is mediated in large part by an immune response directed against the SARS-CoV-2 spike protein, a main target of SARS-CoV-2 vaccines. The spike protein mediates binding and entry into host cells, through binding of the receptor binding domain (RBD) to the host cell receptor angiotensin-converting enzyme 2 (ACE2).

### 3. SUMMARY

Provided herein are, inter alia, SARS-CoV-2 spike proteins (e.g., SARS-CoV-2 spike protein immunogens (and immunogenic fragments and/or immunogenic variants thereof)), nucleic acids encoding the same, compositions (e.g., vaccine compositions, pharmaceutical compositions) comprising the SARS-CoV-2 spike proteins (e.g., SARS-CoV-2 spike protein immunogens (and immunogenic fragments and/or immunogenic variants thereof)) or nucleic acids encoding the same, methods of manufacturing, and methods of use including, e.g., methods of preventing, reducing, or treating a SARS-CoV-2 infection, methods of vaccination against a SARS-CoV-2 infection, etc.

In one aspect, provided herein are nucleic acid molecules comprising at least one coding region encoding a SARS-CoV-2 spike protein (e.g., a SARS-CoV-2 spike protein immunogen (or an immunogenic fragment and/or immunogenic variant thereof)), wherein the amino acid sequence of the encoded SARS-CoV-2 spike protein (e.g., the SARS-CoV-2 spike protein immunogen (or an immunogenic fragment and/or immunogenic variant thereof)), comprises at least one set of amino acid substitutions set forth in Table 2 or Table 4.

In some embodiments, the amino acid sequence of the encoded SARS-CoV-2 spike protein (e.g., the SARS-CoV-2 spike protein immunogen (or an immunogenic fragment and/or immunogenic variant thereof)) comprises a SARS-CoV-2 spike protein receptor binding domain (RBD). In some embodiments, the amino acid sequence of the encoded SARS-CoV-2 spike protein (e.g., the SARS-CoV-2 spike protein immunogen (or an immunogenic fragment and/or immunogenic variant thereof)) consists of a SARS-CoV-2 spike protein receptor binding domain (RBD).

In some embodiments, the amino acid sequence of the encoded SARS-CoV-2 spike protein (e.g., the SARS-CoV-2 spike protein immunogen (or an immunogenic fragment and/or immunogenic variant thereof)) comprises a fragment of a full-length SARS-CoV-2 spike protein that retains the ability to bind ACE2. In some embodiments, the amino acid sequence of the encoded SARS-CoV-2 spike protein (e.g., the SARS-CoV-2 spike protein immunogen (or an immunogenic fragment and/or immunogenic variant thereof)) consists of a fragment of a full-length SARS-CoV-2 spike protein that retains the ability to bind ACE2.

In some embodiments, the amino acid sequence of the encoded SARS-CoV-2 spike protein (e.g., the SARS-CoV-2 spike protein immunogen (or immunogenic fragment and/or immunogenic variant thereof)) comprises a full-length SARS-CoV-2 spike protein. In some embodiments, the amino acid sequence of the encoded SARS-CoV-2 spike protein (e.g., the SARS-CoV-2 spike protein immunogen (or immunogenic fragment and/or immunogenic variant thereof)) consist of a full-length SARS-CoV-2 spike protein.

In some embodiments, the amino acid sequence of the encoded SARS-CoV-2 spike protein (e.g., the SARS-CoV-2 spike protein immunogen (or immunogenic fragment and/or immunogenic variant thereof)) comprises at least one set of amino acid substitutions set forth in Table 2.

In some embodiments, the amino acid sequence of the encoded SARS-CoV-2 spike protein (e.g., the SARS-CoV-2 spike protein immunogen (or immunogenic fragment and/or immunogenic variant thereof)) comprises at least 10, 15, 20, 30, 40, 50, 60, 70, 80, 90, 100, 150, 200, 300, 400, 500, 600, 700, 800, 900, 1000, 1100, 1200, or 1300 amino acids. In some embodiments, the amino acid sequence of the encoded SARS-CoV-2 spike protein (e.g., the SARS-CoV-2 spike protein immunogen (or immunogenic fragment and/or immunogenic variant thereof)) comprises from about 10-15, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 10-1100, 10-1200, or 10-1300 amino acids.

In some embodiments, other than the at least one set of amino acid substitutions set forth in Table 2, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, 1200, or 1300 amino acids set forth in any one of SEQ ID NOS: 1-10 (e.g., any one of SEQ ID NOS: 1-5).

In some embodiments, other than the at least one set of amino acid substitutions set forth in Table 2, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-10 (e.g., any one of SEQ ID NOS: 1-5).

In some embodiments, the amino acid sequence of the encoded SARS-CoV-2 spike protein (e.g., the SARS-CoV-2 spike protein immunogen (or immunogenic fragment and/or immunogenic variant thereof)) comprises a plurality of sets of amino acid substitutions set forth in Table 2. In some embodiments, the amino acid sequence of the encoded SARS-CoV-2 spike protein (e.g., the SARS-CoV-2 spike protein immunogen (or immunogenic fragment and/or immunogenic variant thereof)) comprises at least 2, 3, 4, 5, or 6 or more sets of amino acid substitutions set forth in Table 2.

In some embodiments, the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not set forth in Table 2. In some embodiments, the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-10 (e.g., any one of SEQ ID NOS: 1-5).

In some embodiments, the encoded SARS-CoV-2 spike protein (e.g., the SARS-CoV-2 spike protein immunogen (or immunogenic fragment and/or immunogenic variant thereof)) is stabilized in a prefusion state. In some embodiments, the amino acid sequence of the encoded SARS-CoV-2 spike protein (e.g., the SARS-CoV-2 spike protein immunogen (or immunogenic fragment and/or immunogenic variant thereof)) comprises at least one amino acid variation relative to the amino acid sequence set forth in any one of SEQ ID NOS: 1-2, that stabilizes the SARS-CoV-2 spike protein (e.g., the SARS-CoV-2 spike protein immunogen (or immunogenic fragment and/or immunogenic variant thereof)) in a prefusion state. In some embodiments, the amino acid sequence of the encoded SARS-CoV-2 spike protein (e.g., the SARS-CoV-2 spike protein immunogen (or immunogenic fragment and/or immunogenic variant thereof)) comprises a proline at amino acid position 986 and/or a proline at amino acid position 987, amino acid numbering relative to the amino acid positions set forth in SEQ ID NO: 1.

In some embodiments, the encoded SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site. In some embodiments, the amino acid sequence of the encoded SARS-CoV-2 spike protein (e.g., the SARS-CoV-2 spike protein immunogen (or immunogenic fragment and/or immunogenic variant thereof)) comprises the addition of one or more non-naturally occurring glycosylation motif comprising a non-naturally occurring glycosylation site relative to the amino acid sequence set forth in any one of SEQ ID NOS: 1-10. In some embodiments, the non-naturally occurring glycosylation site is an N-glycosylation site.

In some embodiments, the amino acid sequence of encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of a protein set forth in Table 3.

In some embodiments, the amino acid sequence of encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 11-28. In some embodiments, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 11-16. In some embodiments, the amino acid sequence of encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 17-22. In some embodiments, the amino acid sequence of encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 23-28.

In some embodiments, the amino acid sequence of the encoded SARS-CoV-2 spike protein (e.g., the SARS-CoV-2 spike protein immunogen (or immunogenic fragment and/or immunogenic variant thereof)) comprises at least one set of amino acid substitutions set forth in Table 4.

In some embodiments, other than the at least one set of amino acid substitutions set forth in Table 4, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, 1200, or 1300 amino acids set forth in any one of SEQ ID NOS: 1-10 (e.g., any one of SEQ ID NOS: 6-10).

In some embodiments, other than the at least one set of amino acid substitutions set forth in Table 4, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one set forth in any one of SEQ ID NOS: 1-10 (e.g., any one of SEQ ID NOS: 6-10).

In some embodiments, the amino acid sequence of the encoded SARS-CoV-2 spike protein (e.g., the SARS-CoV-2 spike protein immunogen (or immunogenic fragment and/or immunogenic variant thereof)) comprises a plurality of sets of amino acid substitutions set forth in Table 4. In some embodiments, the amino acid sequence of the encoded SARS-CoV-2 spike protein (e.g., the SARS-CoV-2 spike protein immunogen (or immunogenic fragment and/or immunogenic variant thereof)) comprises at least 2, 3, 4, 5, or 6 or more sets of amino acid substitutions set forth in Table 4.

In some embodiments, the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not set forth in Table 4. In some embodiments, the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 1-10 (e.g., any one of SEQ ID NOS: 6-10).

In some embodiments, the amino acid sequence of encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of a protein set forth in Table 5.

In some embodiments, the amino acid sequence of encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 29-76. In some embodiments, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 29-44. In some embodiments, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 45-60. In some embodiments, the amino acid sequence of encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 61-76.

In some embodiments, the amino acid sequence of the encoded SARS-CoV-2 spike protein (e.g., the SARS-CoV-2 spike protein immunogen (or immunogenic fragment and/or immunogenic variant thereof)) comprises at least one set of amino acid substitutions set forth in Table 2 and at least one set of amino acid substitutions set forth in Table 4.

In some embodiments, other than the at least one set of amino acid substitutions set forth in Table 2 and the at least one set of amino acid substitutions set forth in Table 4, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-10.

In some embodiments, the amino acid sequence of the encoded SARS-CoV-2 spike protein (e.g., the SARS-CoV-2 spike protein immunogen (or immunogenic fragment and/or immunogenic variant thereof)) comprises a plurality of sets of amino acid substitutions set forth in Table 2. In some embodiments, the amino acid sequence of the encoded SARS-CoV-2 spike protein (e.g., the SARS-CoV-2 spike protein immunogen (or immunogenic fragment and/or immunogenic variant thereof)) comprises at least 2, 3, 4, 5, or 6 or more sets of amino acid substitutions set forth in Table 2.

In some embodiments, the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not set forth in Table 2.

In some embodiments, the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-5.

In some embodiments, the amino acid sequence of the encoded SARS-CoV-2 spike protein (e.g., the SARS-CoV-2 spike protein immunogen (or immunogenic fragment and/or immunogenic variant thereof)) comprises a plurality of sets of amino acid substitutions set forth in Table 4. In some embodiments, the amino acid sequence of the encoded SARS-CoV-2 spike protein (e.g., the SARS-CoV-2 spike protein immunogen (or immunogenic fragment and/or immunogenic variant thereof)) comprises at least 2, 3, 4, 5, or 6 or more sets of amino acid substitutions set forth in Table 4.

In some embodiments, the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not set forth in Table 4.

In some embodiments, the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 6-10.

In some embodiments, the amino acid sequence of the encoded SARS-CoV-2 spike protein (e.g., the SARS-CoV-2 spike protein immunogen (or the immunogenic fragment and/or immunogenic variant thereof)) comprises an inactive furin cleavage site. In some embodiments, the amino acid sequence of the encoded SARS-CoV-2 spike protein (e.g., the SARS-CoV-2 spike protein immunogen (or the immunogenic fragment and/or immunogenic variant thereof)) comprises at least one amino acid variation in the furin cleavage site that inactivates the furin cleavage site.

In some embodiments, the encoded SARS-CoV-2 spike protein is an immunogen (or an immunogenic fragment and/or immunogenic variant thereof).

In some embodiments, the nucleic acid molecule is an RNA molecule, a DNA molecule, or a DNA/RNA molecule. In some embodiments, the nucleic acid molecule is an RNA molecule. In some embodiments, the RNA molecule is a translatable RNA molecule. In some embodiments, the RNA molecule is a messenger RNA (mRNA) molecule. In some embodiments, the RNA molecule is a circular RNA molecule.

In some embodiments, the nucleotide sequence of the nucleic acid molecule is codon optimized. In some embodiments, the nucleic acid molecule comprises at least one modified nucleotide. In some embodiments, the nucleic acid molecule comprises N1-methyl-pseudouridine, cytosine, adenine, and guanine.

In some embodiments, the nucleic acid molecule comprises a heterologous 5′-untranslated region (UTR), 3′-UTR, or both a 5′-UTR and 3′-UTR. In some embodiments, the nucleic acid molecule comprises a poly(A) sequence. In some embodiments, the nucleic acid molecule comprises a 5′cap structure.

In some embodiments, the nucleic acid molecule further encodes a heterologous protein.

In some embodiments, the nucleic acid molecule encodes a signal peptide. In some embodiments, the nucleic acid molecule encodes a homologous or heterologous signal peptide. In some embodiments, the nucleic acid molecule does not encode a signal peptide.

In one aspect, provided herein are vectors comprising a nucleic acid molecule described herein. In some embodiments, the vector is a non-viral vector (e.g., a plasmid) or a viral vector.

In one aspect, provided herein are conjugates comprising a nucleic acid molecule described herein operably connected (e.g., directly or indirectly (e.g., via a linker)) to a heterologous moiety (e.g., a heterologous protein).

In one aspect, provided herein are compositions comprising at least one nucleic acid molecule described herein, a vector described herein, or a conjugate described herein.

In some embodiments, the composition comprises a plurality of nucleic acid molecules described herein, wherein the amino acid sequence of each of the SARS-CoV-2 spike proteins (e.g., SARS-CoV-2 spike protein immunogens (or immunogenic fragments and/or immunogenic variants thereof)) encoded by each nucleic acid molecule of the plurality is different. In some embodiments, the plurality comprises at least two nucleic acid molecules described herein, wherein the amino acid sequence of each of the SARS-CoV-2 spike proteins (e.g., SARS-CoV-2 spike protein immunogens (or immunogenic fragments and/or immunogenic variants thereof)) encoded by of each of the at least 2 nucleic acid molecules comprises a different set of amino acid substitutions set forth in Table 2 or Table 4 or a different combination of sets of amino acid substitutions set forth in Table 2 or Table 4.

In some embodiments, the composition further comprises a nucleic acid molecule encoding a SARS-CoV-2 spike protein (e.g., the SARS-CoV-2 spike protein immunogen (or the immunogenic fragment and/or immunogenic variant thereof)) that does not comprise a set of amino acid substitutions set forth in Table 2 or Table 4.

In some embodiments, the nucleic acid molecule(s) or the one or more vectors are formulated in one or more carrier. In some embodiments, the composition is a pharmaceutical composition comprising a pharmaceutically acceptable excipient or a vaccine composition. In some embodiments, the composition further comprising an adjuvant.

In some embodiments, the nucleic acid molecule(s) are comprised in one or more vectors.

In some embodiments, the composition further comprises a carrier. In some embodiments, the carrier is a lipid nanoparticle (LNP), liposome, lipoplex, or nanoliposome. In some embodiments, the carrier is an LNP. In some embodiments, the LNP comprises a cationic lipid, a neutral lipid, a cholesterol, and/or a PEG lipid. In some embodiments, the LNP has a mean particle size of between 80 nm and 160 nm.

In one aspect, provided herein are compositions comprising (a) at least one nucleic acid molecule comprising at least one set of amino acid substitutions set forth in Table 2; (b) at least one nucleic acid molecule comprising at least one set of amino acid substitutions set forth in Table 4; and/or (c) at least one nucleic acid molecule comprising at least one set of amino acid substitutions set forth in Table 2 and at least one set of amino acid substitutions set forth in Table 4.

In some embodiments, the composition further comprises a nucleic acid molecule encoding a SARS-CoV-2 spike protein (e.g., the SARS-CoV-2 spike protein immunogen (or the immunogenic fragment and/or immunogenic variant thereof)) that does not comprise a set of amino acid substitutions set forth in Table 2 or Table 4.

In some embodiments, the nucleic acid molecule(s) or the one or more vectors are formulated in one or more carrier. In some embodiments, the composition is a pharmaceutical composition comprising a pharmaceutically acceptable excipient or a vaccine composition. In some embodiments, the composition further comprising an adjuvant.

In some embodiments, the nucleic acid molecule(s) are comprised in one or more vectors.

In some embodiments, the composition further comprises a carrier. In some embodiments, the carrier is a lipid nanoparticle (LNP), liposome, lipoplex, or nanoliposome. In some embodiments, the carrier is an LNP. In some embodiments, the LNP comprises a cationic lipid, a neutral lipid, a cholesterol, and/or a PEG lipid. In some embodiments, the LNP has a mean particle size of between 80 nm and 160 nm.

In one aspect, provided herein are SARS-CoV-2 spike protein (e.g., a SARS-CoV-2 spike protein immunogen (or an immunogenic fragment and/or immunogenic variant thereof)), wherein the amino acid sequence of the SARS-CoV-2 spike protein (e.g., the SARS-CoV-2 spike protein immunogen (or immunogenic fragment and/or immunogenic variant thereof)), comprises at least one set of amino acid substitutions set forth in Table 2 or Table 4.

In some embodiments, the amino acid sequence of the SARS-CoV-2 spike protein (e.g., the SARS-CoV-2 spike protein immunogen (or the immunogenic fragment and/or immunogenic variant thereof)) comprises a SARS-CoV-2 spike protein receptor binding domain (RBD). In some embodiments, the amino acid sequence of the SARS-CoV-2 spike protein (e.g., the SARS-CoV-2 spike protein immunogen (or the immunogenic fragment and/or immunogenic variant thereof)) consists of a SARS-CoV-2 spike protein receptor binding domain (RBD).

In some embodiments, the amino acid sequence of the SARS-CoV-2 spike protein (e.g., the SARS-CoV-2 spike protein immunogen (or the immunogenic fragment and/or immunogenic variant thereof)) comprises a fragment of a full-length SARS-CoV-2 spike protein that retains the ability to bind ACE2. In some embodiments, the amino acid sequence of the SARS-CoV-2 spike protein (e.g., the SARS-CoV-2 spike protein immunogen (or the immunogenic fragment and/or immunogenic variant thereof)) consists of a fragment of a full-length SARS-CoV-2 spike protein that retains the ability to bind ACE2.

In some embodiments, the amino acid sequence of the SARS-CoV-2 spike protein (e.g., the SARS-CoV-2 spike protein immunogen (or the immunogenic fragment and/or immunogenic variant thereof)) comprises a full-length SARS-CoV-2 spike protein. In some embodiments, the amino acid sequence of the SARS-CoV-2 spike protein (e.g., the SARS-CoV-2 spike protein immunogen (or the immunogenic fragment and/or immunogenic variant thereof)) consists of a full-length SARS-CoV-2 spike protein.

In some embodiments, the amino acid sequence of the SARS-CoV-2 spike protein (e.g., the SARS-CoV-2 spike protein immunogen (or the immunogenic fragment and/or immunogenic variant thereof)) comprises at least one set of amino acid substitutions set forth in Table 2.

In some embodiments, the amino acid sequence of the SARS-CoV-2 spike protein (e.g., the SARS-CoV-2 spike protein immunogen (or the immunogenic fragment and/or immunogenic variant thereof)) comprises at least 10, 15, 20, 30, 40, 50, 60, 70, 80, 90, 100, 150, 200, 300, 400, 500, 600, 700, 800, 900, 1000, 1100, 1200, or 1300 amino acids.

In some embodiments, the amino acid sequence of the SARS-CoV-2 spike protein (e.g., the SARS-CoV-2 spike protein immunogen (or the immunogenic fragment and/or immunogenic variant thereof)) comprises from about 10-15, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 10-1100, 10-1200, or 10-1300 amino acids.

In some embodiments, other than the at least one set of amino acid substitutions, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, 1200, or 1300 amino acids set forth in any one of SEQ ID NOS: 1-5.

In some embodiments, other than the at least one set of amino acid substitutions set forth in Table 2, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-5.

In some embodiments, the amino acid sequence of the SARS-CoV-2 spike protein (e.g., the SARS-CoV-2 spike protein immunogen (or the immunogenic fragment and/or immunogenic variant thereof)) comprises a plurality of sets of amino acid substitutions set forth in Table 2. In some embodiments, the amino acid sequence of the SARS-CoV-2 spike protein (e.g., the SARS-CoV-2 spike protein immunogen (or the immunogenic fragment and/or immunogenic variant thereof)) comprises at least 2, 3, 4, 5, or 6 sets of amino acid substitutions set forth in Table 2. In some embodiments, the amino acid sequence of the SARS-CoV-2 spike protein (e.g., the SARS-CoV-2 spike protein immunogen (or the immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not set forth in Table 2. In some embodiments, the amino acid sequence of the SARS-CoV-2 spike protein (e.g., the SARS-CoV-2 spike protein immunogen (or the immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-5.

In some embodiments, the SARS-CoV-2 spike protein (e.g., the SARS-CoV-2 spike protein immunogen (or the immunogenic fragment and/or immunogenic variant thereof)) is stabilized in a prefusion state. In some embodiments, the amino acid sequence of the SARS-CoV-2 spike protein (e.g., the SARS-CoV-2 spike protein immunogen (or the immunogenic fragment and/or immunogenic variant thereof)) comprises at least one amino acid variation relative to the amino acid sequence set forth in SEQ ID NO: 1 or SEQ ID NO: 2 that stabilizes the SARS-CoV-2 spike protein (e.g., the SARS-CoV-2 spike protein immunogen (or the immunogenic fragment and/or immunogenic variant thereof)) in a prefusion state. In some embodiments, the amino acid sequence of the SARS-CoV-2 spike protein (e.g., the SARS-CoV-2 spike protein immunogen (or the immunogenic fragment and/or immunogenic variant thereof)) comprises a proline at amino acid position 986 and/or a proline at amino acid position 987, amino acid numbering relative to the amino acid positions set forth in SEQ ID NO: 1.

In some embodiments, the SARS-CoV-2 spike protein (e.g., the SARS-CoV-2 spike protein immunogen (or the immunogenic fragment and/or immunogenic variant thereof)) comprises one or more non-naturally occurring glycosylation motif comprising a non-naturally occurring glycosylation site. In some embodiments, the amino acid sequence of the SARS-CoV-2 spike protein (e.g., the SARS-CoV-2 spike protein immunogen (or the immunogenic fragment and/or immunogenic variant thereof)) comprises the addition of one or more non-naturally occurring glycosylation motif comprising a non-naturally occurring glycosylation site relative to the amino acid sequence set forth in any one of SEQ ID NOS: 1-10. In some embodiments, the non-naturally occurring glycosylation site is an N-glycosylation site.

In some embodiments, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of a protein set forth in Table 3.

In some embodiments, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 11-28. In some embodiments, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 11-16. In some embodiments, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 17-22. In some embodiments, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 23-28.

In some embodiments, the amino acid sequence of the SARS-CoV-2 spike protein (e.g., the SARS-CoV-2 spike protein immunogen (or the immunogenic fragment and/or immunogenic variant thereof)) comprises at least one set of amino acid substitutions set forth in Table 4.

In some embodiments, other than the at least one set of amino acid substitutions the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, 1200, or 1300 amino acids set forth in any one of SEQ ID NOS: 6-10.

In some embodiments, other than the at least one set of amino acid substitutions the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 6-10.

In some embodiments, the amino acid sequence of the SARS-CoV-2 spike protein (e.g., the SARS-CoV-2 spike protein immunogen (or the immunogenic fragment and/or immunogenic variant thereof)) comprises a plurality of sets of amino acid substitutions set forth in Table 4. In some embodiments, the amino acid sequence of the SARS-CoV-2 spike protein (e.g., the SARS-CoV-2 spike protein immunogen (or the immunogenic fragment and/or immunogenic variant thereof)) comprises at least 2, 3, 4, 5, or 6 sets of amino acid substitutions set forth in Table 4.

In some embodiments, the amino acid sequence of the SARS-CoV-2 spike protein (e.g., the SARS-CoV-2 spike protein immunogen (or the immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4. In some embodiments, the amino acid sequence of the SARS-CoV-2 spike protein (e.g., the SARS-CoV-2 spike protein immunogen (or the immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 1-5.

In some embodiments, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of a protein set forth in Table 5.

In some embodiments, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 29-76. In some embodiments, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 29-44. In some embodiments, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 45-60. In some embodiments, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 61-76.

In some embodiments, the amino acid sequence of the SARS-CoV-2 spike protein (e.g., the SARS-CoV-2 spike protein immunogen (or the immunogenic fragment and/or immunogenic variant thereof)) comprises at least one set of amino acid substitutions set forth in Table 2 and at least one set of amino acid substitutions set forth in Table 4.

In some embodiments, other than the at least one set of amino acid substitutions, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, 1200, or 1300 amino acids set forth in any one of SEQ ID NOS: 1-10.

In some embodiments, other than the at least one set of amino acid substitutions set forth in Table 2 and the at least one set of amino acid substitutions set forth in Table 4, the amino acid sequence of the SARS-CoV-2 spike protein (e.g., the SARS-CoV-2 spike protein immunogen (or the immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-10.

In some embodiments, the amino acid sequence of the SARS-CoV-2 spike protein (e.g., the SARS-CoV-2 spike protein immunogen (or the immunogenic fragment and/or immunogenic variant thereof)) comprises a plurality of sets of amino acid substitutions set forth in Table 2. In some embodiments, the amino acid sequence of the SARS-CoV-2 spike protein (e.g., the SARS-CoV-2 spike protein immunogen (or the immunogenic fragment and/or immunogenic variant thereof)) comprises at least 2, 3, 4, 5, or 6 sets of amino acid substitutions set forth in Table 2.

In some embodiments, the amino acid sequence of the SARS-CoV-2 spike protein (e.g., the SARS-CoV-2 spike protein immunogen (or the immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not set forth in Table 2. In some embodiments, the amino acid sequence of the SARS-CoV-2 spike protein (e.g., the SARS-CoV-2 spike protein immunogen (or the immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-5.

In some embodiments, the amino acid sequence of the SARS-CoV-2 spike protein (e.g., the SARS-CoV-2 spike protein immunogen (or the immunogenic fragment and/or immunogenic variant thereof)) comprises a plurality of sets of amino acid substitutions set forth in Table 4. In some embodiments, the amino acid sequence of the SARS-CoV-2 spike protein (e.g., the SARS-CoV-2 spike protein immunogen (or the immunogenic fragment and/or immunogenic variant thereof)) comprises at least 2, 3, 4, 5, 6, 7, 8, 9, 10, 11, 12, 13, 14, 15, or 16 sets of amino acid substitutions set forth in Table 4.

In some embodiments, the amino acid sequence of the SARS-CoV-2 spike protein (e.g., the SARS-CoV-2 spike protein immunogen (or the immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4. In some embodiments, the amino acid sequence of the SARS-CoV-2 spike protein (e.g., the SARS-CoV-2 spike protein immunogen (or the immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 6-10.

In some embodiments, the amino acid sequence of the SARS-CoV-2 spike protein (e.g., the SARS-CoV-2 spike protein immunogen (or the immunogenic fragment and/or immunogenic variant thereof)) comprises an inactive furin cleavage site. In some embodiments, the amino acid sequence of the SARS-CoV-2 spike protein (e.g., the SARS-CoV-2 spike protein immunogen (or the immunogenic fragment and/or immunogenic variant thereof)) comprises at least one amino acid variation in the furin cleavage site that inactivates the furin cleavage site.

In some embodiments, the SARS-CoV-2 spike protein (e.g., the SARS-CoV-2 spike protein immunogen (or the immunogenic fragment and/or immunogenic variant thereof)) further comprises a heterologous protein.

In some embodiments, the SARS-CoV-2 spike protein (e.g., the SARS-CoV-2 spike protein immunogen (or the immunogenic fragment and/or immunogenic variant thereof)) comprises a signal peptide. In some embodiments, the SARS-CoV-2 spike protein (e.g., the SARS-CoV-2 spike protein immunogen (or the immunogenic fragment and/or immunogenic variant thereof)) comprises a homologous or heterologous signal peptide. In some embodiments, the SARS-CoV-2 spike protein (e.g., the SARS-CoV-2 spike protein immunogen (or the immunogenic fragment and/or immunogenic variant thereof)) does not comprise a signal peptide.

In some embodiments, the SARS-CoV-2 spike protein is an immunogen (or an immunogenic fragment and/or immunogenic variant thereof).

In one aspect, provided herein are fusion proteins comprising a SARS-CoV-2 spike protein (e.g., the SARS-CoV-2 spike protein immunogen (or immunogenic fragment and/or immunogenic variant thereof)) described herein operably connected (e.g., directly or indirectly (e.g., via a linker)) to a heterologous protein.

In one aspect, provided herein are conjugates comprising a SARS-CoV-2 spike protein (e.g., the SARS-CoV-2 spike protein immunogen (or immunogenic fragment and/or immunogenic variant thereof)) described herein operably connected (e.g., directly or indirectly (e.g., via a linker)) to a heterologous moiety.

In one aspect, provided herein are compositions comprising at least one SARS-CoV-2 spike protein (e.g., at least one of the SARS-CoV-2 spike protein immunogen (or immunogenic fragment and/or immunogenic variant thereof)) described herein, a fusion protein described herein, a conjugate described herein, or a nucleic acid molecule described herein.

In some embodiments, the composition comprises a plurality of SARS-CoV-2 spike proteins (e.g., a plurality of SARS-CoV-2 spike protein immunogens (or immunogenic fragments and/or immunogenic variants thereof)) described herein, wherein the amino acid sequence of each of the plurality of SARS-CoV-2 spike proteins (e.g., the SARS-CoV-2 spike protein immunogens (or immunogenic fragments and/or immunogenic variants thereof)) comprises a different set of amino acid substitutions set forth in Table 2 or Table 4 or a different combination of sets of amino acid substitutions set forth in Table 2 or Table 4.

In some embodiments, the composition further comprises a SARS-CoV-2 spike proteins (e.g., the SARS-CoV-2 spike protein immunogen (or the immunogenic fragment and/or immunogenic variant thereof)) that does not comprise a set of amino acid substitutions set forth in Table 2 or Table 4.

In some embodiments, the composition is a pharmaceutical composition comprising a pharmaceutically acceptable excipient. In some embodiments, the composition further comprises an adjuvant.

In some embodiments, the SARS-CoV-2 spike proteins (e.g., the SARS-CoV-2 spike protein immunogen (or the immunogenic fragment and/or immunogenic variant thereof)) are formulated in one or more carrier. In some embodiments, the carrier is a lipid nanoparticle (LNP), liposome, lipoplex, or nanoliposome. In some embodiments, the carrier is an LNP. In some embodiments, the LNP comprises a cationic lipid, a neutral lipid, a cholesterol, and/or a PEG lipid. In some embodiments, the LNP has a mean particle size of between 80 nm and 160 nm.

In one aspect, provided herein are compositions comprising (a) at least one SARS-CoV-2 spike proteins (e.g., the SARS-CoV-2 spike protein immunogen (or the immunogenic fragment and/or immunogenic variant thereof)) described herein that comprises at least one set of amino acid substitutions set forth in Table 2; (b) at least one SARS-CoV-2 spike proteins (e.g., the SARS-CoV-2 spike protein immunogen (or the immunogenic fragment and/or immunogenic variant thereof)) described herein that comprises at least one set of amino acid substitutions set forth in Table 4; and/or (c) at least one SARS-CoV-2 spike proteins (e.g., the SARS-CoV-2 spike protein immunogen (or the immunogenic fragment and/or immunogenic variant thereof)) described herein that comprises at least one set of amino acid substitutions set forth in Table 2 and at least one set of amino acid substitutions set forth in Table 4.

In some embodiments, the composition further comprises a SARS-CoV-2 spike proteins (e.g., the SARS-CoV-2 spike protein immunogen (or the immunogenic fragment and/or immunogenic variant thereof)) that does not comprise a set of amino acid substitutions set forth in Table 2 or Table 4.

In some embodiments, the composition is a pharmaceutical composition comprising a pharmaceutically acceptable excipient. In some embodiments, the composition further comprises an adjuvant.

In some embodiments, the SARS-CoV-2 spike proteins (e.g., the SARS-CoV-2 spike protein immunogen (or the immunogenic fragment and/or immunogenic variant thereof)) are formulated in one or more carrier. In some embodiments, the carrier is a lipid nanoparticle (LNP), liposome, lipoplex, or nanoliposome. In some embodiments, the carrier is an LNP. In some embodiments, the LNP comprises a cationic lipid, a neutral lipid, a cholesterol, and/or a PEG lipid. In some embodiments, the LNP has a mean particle size of between 80 nm and 160 nm.

In one aspect, provided herein are nucleic acid molecules encoding a SARS-CoV-2 spike protein (e.g., the SARS-CoV-2 spike protein immunogen (or the immunogenic fragment and/or immunogenic variant thereof)) described herein.

In one aspect, provided herein are SARS-CoV-2 spike proteins (e.g., a SARS-CoV-2 spike protein immunogen (or an immunogenic fragment and/or immunogenic variant thereof)) encoded by a nucleic acid molecule described herein.

In one aspect, provided herein are carriers comprising a nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a vaccine composition described herein, or a pharmaceutical composition described herein.

In some embodiments, the carrier is a lipid nanoparticle (LNP), liposome, lipoplex, or nanoliposome. In some embodiments, the carrier is an LNP. In some embodiments, the LNP comprises a cationic lipid, a neutral lipid, a cholesterol, and/or a PEG lipid. In some embodiments, the LNP has a mean particle size of between 80 nm and 160 nm.

In one aspect, provided herein are vaccine compositions comprising a nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, or a pharmaceutical composition described herein.

In some embodiments, the vaccine composition is a prime vaccine composition. In some embodiments, the vaccine composition is a boost vaccine composition. In some embodiments, the vaccine composition is a prime vaccine composition and a boost vaccine composition. In some embodiments, the vaccine composition can be utilized as a prime vaccine composition and/or a booster vaccine composition in a homologous or heterologous prime boost vaccine regimen. In some embodiments, the vaccine composition further comprises an adjuvant.

In one aspect, provided herein are vaccine compositions comprising a messenger ribonucleic acid (mRNA) encoding a SARS-CoV-2 spike protein (e.g., a SARS-CoV-2 spike protein immunogen (or an immunogenic fragment and/or immunogenic variant thereof)) that comprises at least one amino acid substitution set forth in Table 2 or Table 4, formulated in a lipid nanoparticle, the vaccine composition having the following characteristics: (a) the LNPs comprise a cationic lipid, a neutral lipid, a cholesterol, and a PEG lipid, (b) the LNPs have a mean particle size of between 80 nm and 160 nm, and (c) the mRNA comprises: (i) a 5′-cap structure; (ii) a 5′-UTR; (iii) N1-methyl-pseudouridine, cytosine, adenine, and guanine; (iv) a 3′-UTR; and (v) a poly-A region. In some embodiments, the vaccine composition further comprises an adjuvant.

In one aspect, provided herein are pharmaceutical compositions comprising a nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, or a vaccine composition described herein, and a pharmaceutically acceptable excipient. In some embodiments, the pharmaceutical composition further comprises an adjuvant.

In one aspect, provided herein are pharmaceutical compositions comprising a messenger ribonucleic acid (mRNA) encoding a SARS-CoV-2 spike protein (e.g., a SARS-CoV-2 spike protein immunogen (or an immunogenic fragment and/or immunogenic variant thereof)) that comprises at least one amino acid substitution set forth in Table 2 or Table 4, formulated in a lipid nanoparticle, the pharmaceutical composition having the following characteristics: (a) the LNPs comprise a cationic lipid, a neutral lipid, a cholesterol, and a PEG lipid, (b) the LNPs have a mean particle size of between 80 nm and 160 nm, and (c) the mRNA comprises: (i) a 5′-cap structure; (ii) a 5′-UTR; (iii) N1-methyl-pseudouridine, cytosine, adenine, and guanine; (iv) a 3′-UTR; and (v) a poly-A region. In some embodiments, the pharmaceutical composition further comprises an adjuvant.

In one aspect, provided herein are host cells comprising a nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a composition described herein, a carrier described herein, a vaccine composition described herein, or a pharmaceutical composition described herein.

In one aspect, provided herein are kits comprising a nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, a vaccine composition described herein, or a pharmaceutical composition described herein.

In some embodiments, the kit comprises instructions for use of the nucleic acid molecule, vector, protein (or immunogenic fragment or immunogenic variant thereof), conjugate, fusion protein, carrier, composition, vaccine composition, or pharmaceutical composition.

In one aspect, provided herein are methods of delivering a nucleic acid molecule, vector, protein (or immunogenic fragment or immunogenic variant thereof), conjugate, fusion protein, host cell, carrier, composition, vaccine composition, or pharmaceutical composition to a subject in need thereof, the method comprising administering to the subject a nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, a vaccine composition described herein, or a pharmaceutical composition described herein, to thereby deliver the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the host cell, the carrier, the composition, the vaccine composition, or the pharmaceutical composition to the subject.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

In one aspect, provided herein are methods of inducing or enhancing an immune response in a subject in need thereof, the method comprising administering to a nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, a vaccine composition described herein, or a pharmaceutical composition described herein, to thereby induce or enhance an immune response the subject.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

In one aspect, provided herein are methods of preventing, ameliorating, or treating a SARS-CoV-2 infection in a subject in need thereof, the method comprising administering to the subject a nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, a vaccine composition described herein, or a pharmaceutical composition described herein, to thereby prevent, ameliorate, or treat the SARS-CoV-2 infection the subject.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

In one aspect, provided herein are methods of vaccinating a subject against SARS-CoV-2, the method comprising administering to the subject a nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, a vaccine composition described herein, or a pharmaceutical composition described herein, to thereby vaccinate the subject against SARS-CoV-2.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

In one aspect, provided herein are methods of vaccinating a subject against SARS-CoV-2, the method comprising administering to the subject (a) an mRNA molecule (e.g., an mRNA molecule described herein) encoding the SARS-CoV-2 spike protein (e.g., a SARS-CoV-2 spike protein or polypeptide immunogen (or immunogenic fragment and/or immunogenic variant thereof)) of any one of claims 99-171 or 186 (or a conjugate or fusion protein thereof), (b) a vector comprising the mRNA molecule, (c) a carrier comprising the mRNA molecule or the vector, (d) a vaccine composition comprising the mRNA molecule, the vector, or the carrier, or (e) a pharmaceutical composition comprising the mRNA molecule, the vector, the carrier, or the vaccine composition, to thereby vaccinate the subject against SARS-CoV-2, to thereby vaccinate the subject against SARS-CoV-2.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

In one aspect, provided herein are methods of vaccinating a subject against SARS-CoV-2, the method comprising administering to the subject a vaccine composition described herein or a pharmaceutical composition described herein, to thereby vaccinate the subject against SARS-CoV-2.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

A nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, a vaccine composition described herein, or a pharmaceutical composition described herein for use in the manufacture of a medicament for the inducement and/or enhancement of an immune response in a subject in need thereof.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

A nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, a vaccine composition described herein, or a pharmaceutical composition described herein for use in the manufacture of a medicament for the prevention, amelioration, and/or treatment of a SARS-CoV-2 infection in a subject in need thereof.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

A nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, a vaccine composition described herein, or a pharmaceutical composition described herein for use in the manufacture of a medicament for the vaccination a subject against SARS-CoV-2.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

A nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, a vaccine composition described herein, or a pharmaceutical composition described herein for use a method of inducing or enhancing an immune response in a subject in need thereof, the method comprising administering the nucleic acid molecule, the protein, the vector, the conjugate, the fusion protein, the host cell, the carrier, the composition, the vaccine composition, or the pharmaceutical composition to the subject in need thereof.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

A nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, a vaccine composition described herein, or a pharmaceutical composition described herein for use in a method of preventing, ameliorating, and/or treating a SARS-CoV-2 infection in a subject in need thereof, the method comprising administering the nucleic acid molecule, the protein, the vector, the conjugate, the fusion protein, the host cell, the carrier, the composition, the vaccine composition, or the pharmaceutical composition to the subject in need thereof.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

A nucleic acid molecule described herein, a protein (e.g., the SARS-CoV-2 spike protein immunogen (or the immunogenic fragment and/or immunogenic variant thereof)) described herein, a vector described herein, a conjugate described herein, a fusion protein described herein, a host cell described herein, a composition described herein, a carrier described herein, a vaccine composition described herein, or a pharmaceutical composition described herein for use in a method of vaccinating a subject against SARS-CoV-2, the method comprising administering the nucleic acid molecule, the protein, the vector, the conjugate, the fusion protein, the host cell, the carrier, the composition, the vaccine composition, or the pharmaceutical composition to the subject in need thereof.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the subject is a human. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

### 4. BRIEF DESCRIPTION OF THE FIGURES

FIGS. 1A-1C depict a sequence alignment of the amino acid sequence of a reference SARS-CoV-2 spike protein Wuhan-Hu-1 Spike reference sequence (SEQ ID NO: 1) and a variant thereof (SEQ ID NO: 6).

### 5. DETAILED DESCRIPTION

SARS-CoV-2 continues to evolve into new variants comprising a variety of amino acid variations, e.g., substitutions, deletions, and insertions. Many of the variations are found in the RBD of the spike protein, which is vital for SARS-CoV-2 entry into the cell. As most of the SARS-CoV-2 vaccines and current antibody therapies target the RBD of the spike protein, this creates the potential for the evolution of SARS-CoV-2 variants that evade vaccine-induced, infection-induced immunity, or current antibody therapies. The inventors have identified sequence variations in the SARS-CoV-2 spike protein (e.g., in the RBD) that e.g., counter SARS-CoV-2 resistance to vaccine induced immunity and/or are potential SARS-CoV-2 variants. Accordingly, novel SARS-CoV-2 spike proteins (e.g., SARS-CoV-2 spike protein immunogens (and immunogenic fragments and/or immunogenic variants thereof)) (or nucleic acid molecules, e.g., mRNAs, encoding such SARS-CoV-2 spike proteins) comprising at least one such sequence variation are good candidates for variant-proof or variant-resistant vaccines against COVID-19. A such, the current disclosure provides, inter alia, novel immunogens for use in pharmaceutical compositions and vaccines to induce a desired immune response against one or more variants of SARS-CoV-2.

TABLE OF CONTENTS

| | |
|---|---|
| 5.1 | Definitions |
| 5.2 | SARS-CoV-2 Spike Proteins (e.g., Immunogens) |
| 5.2.1 | Exemplary Additional Features of SARS-CoV-2 Spike Proteins |
| | (e.g., Immunogens) |
| 5.2.1.1 | Full-Length Spike Proteins and Receptor Binding Fragments |
| 5.2.1.2 | Stabilization |
| 5.2.1.3 | Multimerization (e.g., Trimerization) Domains |
| 5.2.1.4 | Engineered Glycosylation Sites |
| 5.2.1.5 | Hyperglycosylation |
| 5.2.1.6 | Additional Variations |
| 5.2.1.7 | Variations from Different Strains of SARS-CoV-2 |
| 5.3 | Nucleic Acid Molecules |
| 5.3.1 | DNA Molecules |
| 5.3.2 | RNA Molecules |
| 5.4 | Fusions & Conjugates |
| 5.5 | Vaccine Compositions |
| 5.5.1 | Peptide and Protein-Based Vaccines |
| 5.5.2 | Nucleic Acid-Based Vaccines |
| 5.6 | Vectors |
| 5.7 | Carriers |
| 5.7.1 | Lipid Based Carriers/Lipid Nanoformulations |
| 5.7.1.1 | Cationic Lipids (Positively Charged) and Ionizable Lipids |
| 5.7.1.2 | Non-Cationic Lipids (e.g., Phospholipids) |
| 5.7.1.3 | Structural Lipids |
| 5.7.1.4 | Polymers and Polyethylene Glycol (PEG) - Lipids |
| 5.7.1.5 | Percentages of Lipid Nanoformulation Components |
| 5.8 | Pharmaceutical Compositions |
| 5.9 | Adjuvants |
| 5.10 | Host Cells |
| 5.11 | Methods of Making Polypeptides & Proteins |
| 5.12 | Methods of Making Nucleic Acid Molecules |
| 5.13 | Methods of Use |
| 5.13.1 | Methods of Delivery |
| 5.13.2 | Methods of Inducing or Enhancing an Immune Response |
| 5.13.3 | Methods of Preventing, Ameliorating, and/or Treating |
| | Infection |
| 5.13.4 | Methods of Vaccination against SARS-CoV-2 |
| 5.13.5 | Methods of Vaccinating a Subject against SARS-CoV-2 |
| 5.13.6 | Methods of Vaccinating a Subject Utilizing a SARS-CoV-2 |
| | mRNA Vaccine |
| 5.14 | Kits |

### 5.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” refers to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” should be assumed to be within an acceptable error range for that particular value or composition.

Where proteins are described herein, it is understood that polynucleotides (e.g., RNA (e.g., mRNA) or DNA nucleic acid molecules) encoding the protein are also provided herein.

Where proteins, polypeptides, nucleic acid molecules, vectors, carriers, etc. are described herein, it is understood that isolated forms of the proteins, nucleic acid molecules, vectors, carriers, etc. are also provided herein.

Where proteins, polypeptides, nucleic acid molecules, etc. are described herein, it is understood that recombinant forms of the proteins, nucleic acid molecules, etc. are also provided herein.

Where polypeptides or sets of polypeptides are described herein, it is understood that proteins comprising the polypeptides or sets of polypeptides folded into their three-dimensional structure (i.e., tertiary or quaternary structure) are also provided herein and vice versa.

As used herein, the term “adjuvant” refers to a substance that causes stimulation of the immune system of a subject when administered to the subject.

As used herein, the term “administering” refers to the physical introduction of an agent, e.g., a vaccine or therapeutic agent (or a precursor of the therapeutic agent that is metabolized or altered (e.g., translation of a nucleic acid molecule) within the body of the subject to produce the therapeutic agent in vivo) to a subject, using any of the various methods and delivery systems known to those skilled in the art. Administering can also be performed, for example, once, a plurality of times, and/or over one or more extended periods.

As used herein, the term “agent” is used generically to describe any macro or micro molecule. Exemplary moieties include, but are not limited to polypeptides, proteins, peptides, nucleic acid molecules (e.g., DNA, RNA), small molecules, carbohydrates, lipids, synthetic polymers (e.g., polymers of PEG).

As used herein, the term “circular RNA” refers to a translatable RNA molecule that forms a circular structure through covalent or non-covalent bonds.

As used herein, the term “derived from,” with reference to a polynucleotide refers to a polynucleotide that has at least 70% sequence identity to a reference polynucleotide (e.g., a naturally occurring polynucleotide) or a fragment thereof. The term “derived from,” with reference to a polypeptide or protein refers to a polypeptide or protein that comprises an amino acid sequence that has at least 70% sequence identity to the amino acid sequence of a reference polypeptide or protein (e.g., a naturally occurring polypeptide or protein). The term “derived from” as used herein does not denote any specific process or method for obtaining the polynucleotide, polypeptide, or protein. For example, the polynucleotide, polypeptide, or protein can be recombinant produced or chemically synthesized.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology. The term disease includes infection (e.g., a viral (e.g., a SARS-Cov-2 infection), bacterial, fungal, protozoal infection).

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

As used herein, the term “full-length” with reference to a SARS-CoV-2 spike protein refers to a SARS-CoV-2 spike protein, wherein the amino acid sequence of the SARS-CoV-2 spike protein comprises substantially the same number of amino acids as a reference SARS-CoV-2 spike protein.

As used herein, the term “fuse” and grammatical equivalents thereof refer to the operable connection of at least a first polypeptide or protein to a second polypeptide or protein, wherein the first and second polypeptides or proteins are not naturally found operably connected together. For example, the first and second polypeptides or proteins are derived from different proteins. The term fuse encompasses both a direct connection of the at least two polypeptides or proteins through a peptide bond, and the indirect connection through a linker (e.g., a peptide linker).

As used herein, the term “heterologous”, when used to describe a first element in reference to a second element means that the first element and second element do not exist in nature disposed as described. For example, a polypeptide comprising a “heterologous moiety” means a polypeptide that is joined to a moiety (e.g., small molecule, polypeptide, polynucleotide, carbohydrate, lipid, synthetic polymer (e.g., polymers of PEG), etc.) that is not joined to the polypeptide in nature.

As used herein, the term “fusion protein” and grammatical equivalents thereof refers to a protein that comprises at least one polypeptide operably connected to another polypeptide, wherein the first and second polypeptides are different and not naturally found operably connected together. For example, the first and second polypeptides of the fusion protein are each derived from different proteins. The at least two polypeptides of the fusion protein can be directly operably connected through a peptide bond; or can be indirectly operably connected through a linker (e.g., a peptide linker). Therefore, for example, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “glycosylation site” refers to the amino acid residue of a protein or peptide which is the attachment point for a glycan. For example, in N-linked glycosylation, the glycosylation site will be the asparagine residue to which the glycan is attached. For further example, in O-linked glycosylation, the glycosylation site will be the serine or threonine residue to which the glycan is attached. The term “non-naturally occurring glycosylation site” within a protein or peptide refers to a glycosylation site that is not found in a specific protein or peptide in nature. The non-naturally occurring glycosylation site can be generated by creating a non-naturally occurring glycosylation motif, which does not necessarily require varying the amino acid of the glycosylation site. For example, a non-naturally occurring glycosylation site would include a glycosylation site generated from the variation of a N-A-P amino acid sequence of a protein to a N-A-T amino acid sequence (creating an N-linked glycosylation motif) even though the amino acid that mediates attachment of the glycan (N) was not varied.

As used herein, the term “glycosylation motif” refers to the amino acid or sequence of amino acids that comprises the glycosylation site and facilities glycosylation. For example, in N-linked glycosylation, the canonical glycosylation motifs are NXT and NXS, wherein X is any amino acid except for proline and the asparagine (N) residue is the glycosylation site. For further example, in O-linked glycosylation, the glycosylation motif is a S or T residue that also serves as the glycosylation site.

As used herein, the term “half-life extension protein or “half-life extension polypeptide” refers to a polypeptide or protein that when operably connected to another moiety (e.g., a subject moiety) (e.g., a protein), increases the half-life of the subject moiety (e.g., the subject protein) in vivo when administered to a subject (e.g., a human subject). The pharmacokinetic properties of the protein can be evaluated utilizing in vivo models known in the art.

As used herein, the term “heterologous”, when used to describe a first element in reference to a second element means that the first element and second element do not exist in nature disposed as described. For example, a polypeptide comprising a “heterologous moiety” means a polypeptide that is joined to a moiety (e.g., small molecule, polypeptide, nucleic acid molecule, carbohydrate, lipid, synthetic polymer (e.g., polymers of PEG), etc.) that is not joined to the polypeptide in nature.

As used, herein the term “heterologous signal peptide” refers to a signal peptide that is not operably connected to a subject polypeptide or protein in nature. For example, in reference to a polypeptide comprising a signal peptide from human IL-2 operably connected to human IL-12, the human IL-2 signal peptide would constitute a heterologous signal peptide.

As used herein, the term “heterologous prime boost” refers to a prime boost vaccine regimen wherein the prime vaccine composition and the boost (or booster) vaccine composition are different (e.g., the immunogen is different, the form of the immunogen is different (e.g., a nucleic acid (e.g., mRNA) molecule-based vaccine versus a protein-based vaccine), the immunogen is expressed from a different vector (e.g., plasmid, viral vector), the method of delivering the immunogen to the subject is different, etc.).

As used herein, the term “homologous signal peptide” refers to a signal peptide that is operably connected to a subject polypeptide or protein in nature. For example, in reference to a polypeptide comprising a signal peptide from human IL-2 operably connected to human IL-2, the human IL-2 signal peptide would constitute a homologous signal peptide.

As used herein, the term “homologous prime boost” refers to a prime boost vaccine regimen wherein the prime vaccine composition and the boost (or booster) vaccine composition are the same.

As used herein, the term “immunogen” refers to a substance that is capable of inducing an immune response (e.g., an adaptive immune response) in a subject (e.g., a human subject).

As used herein, the term “immunogenic fragment” refers to a fragment of a reference polypeptide or protein that retains an immunogen.

As used herein, the term “immunogenic variant” refers to a variant of a reference polypeptide or protein that retains an immunogen. In some embodiments, the polypeptide or protein comprises at least one but no more than 25%, (e.g., no more than 20%, no more than 15%, no more than 12%, no more than 10%, no more than 8%) amino acid variation (e.g., substitutions, deletions, additions) compared to the amino acid sequence of a reference polypeptide or protein.

As used herein, the term “in combination with” means that two (or more) different agents or treatments are administered to a subject as part of a defined treatment regimen for a particular disease. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen (e.g., a prime-boost vaccine regimen). In some embodiments, administration of two or more agents or treatments in combination is such that the reduction in a symptom, or other parameter related to the disease is greater than what would be observed with one agent or treatment delivered alone or in the absence of the other. The effect of the two treatments can be partially additive, wholly additive, or greater than additive (e.g., synergistic). Sequential or substantially simultaneous administration of each agent can be effected by any appropriate route including, but not limited to, oral routes, intravenous routes, intramuscular routes, and direct absorption through mucous membrane tissues. The agents can be administered by the same route or by different routes.

As used herein, the term “isolated” with reference to an agent (e.g., a polypeptide, protein, or nucleic acid molecule) refers to the agent (e.g., the polypeptide, protein, or nucleic acid molecule) that is substantially free of other cellular components with which it is associated in the natural state.

As used herein, the term “modification” in reference to a nucleic acid sequence refers to a nucleic acid molecule that comprises at least one nucleotide comprising a chemical modification, e.g., a modified sugar moiety, a modified nucleobase, and/or a modified internucleotide linkage, or any combination thereof. Exemplary nucleotide modifications are provided herein, see, e.g., § 5.3 (e.g., § 5.3.2). In certain embodiments of the instant disclosure, inclusion of a deoxynucleotide—which is acknowledged as a naturally occurring form of nucleotide—if present within an RNA molecule (e.g., an mRNA molecule) is considered to constitute a modified nucleotide.

The terms “nucleic acid molecule” and “polynucleotide” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “operably connected” refers to the linkage of two moieties in a functional relationship. For example, a polypeptide is operably connected to another polypeptide when they are linked (either directly or indirectly via a peptide linker) in frame such that both polypeptides are functional (e.g., a fusion protein described herein). Or for example, a transcription regulatory polynucleotide e.g., a promoter, enhancer, or other expression control element is operably connected to a polynucleotide that encodes a protein if it affects the transcription of the polynucleotide that encodes the protein. The term “operably connected” can also refer to the conjugation of a moiety to e.g., a polynucleotide or polypeptide (e.g., the conjugation of a PEG polymer to a protein).

The determination of “percent identity” between two sequences (e.g., peptide or protein (amino acid sequences) or nucleic acid sequences)) can be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance for use in contact with the tissues of human beings and/or non-human animals without excessive toxicity, irritation, allergic response, or other problem or complication, commensurate with a reasonable therapeutic benefit/risk ratio.

As used herein, the term “plurality” means 2 or more (e.g., 3 or more, 4 or more, 5 or more, 6 or more, 7 or more, 9 or more, or 10 or more).

As used herein, the term “poly(A) sequence,” refers to a sequence of adenosine nucleotides, typically located at the 3′-end of a linear RNA (or in a circular RNA), of up to about 1000 adenosine nucleotides. In some embodiments, the poly(A) sequence is essentially homopolymeric, e.g., a poly(A) sequence of e.g., 100 adenosine nucleotides has essentially the length of 100 nucleotides. In other embodiments, the poly(A) sequence may be interrupted by at least one nucleotide different from an adenosine nucleotide, e.g., a poly(A) sequence of e.g., 100 adenosine nucleotides may have a length of more than 100 nucleotides (comprising 100 adenosine nucleotides and in addition said at least one nucleotide—or a stretch of nucleotides—different from an adenosine nucleotide). It has to be understood that “poly(A) sequence” as defined herein typically relates to RNA—however in the context of the invention, the term likewise relates to corresponding sequences in a DNA molecule (e.g. a “poly(T) sequence”).

As used herein, the term “prime boost” refers to a vaccine regimen comprising at least an initial vaccine dose and one or more subsequent vaccine doses. The initial vaccine dose comprises the prime vaccine composition and the one or more subsequent vaccine doses are referred to as boost (or booster) vaccine compositions. Prime boost vaccine regimens can comprise more than one booster (e.g., 2, 3, 4, 5, 6, or more, etc.).

A “prophylactic” treatment is a treatment administered to a subject who does not exhibit signs of a disease or exhibits only early signs for the purpose of decreasing the risk of developing pathology.

As used herein, the term “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” are used interchangeably herein.

As used herein, the term “protein” refers to one or more polypeptides folded into its three-dimensional structure. Where polypeptides are contemplated herein, it should be understood that proteins comprising the polypeptides (i.e., the polypeptides folded unto their three-dimensional structure) are also provided herein.

As used herein, the term “receptor binding domain” or “RBD” in reference to the SARS-CoV-2 spike protein refers to a fragment of the full-length SARS-CoV-2 spike protein required for the SARS-CoV-2 spike protein to bind ACE2. The amino acid sequence of an exemplary reference SARS-CoV-2 spike protein RBD (of the full-length immature SARS-CoV-2 spike protein set forth in SEQ ID NO: 1) is set forth in SEQ ID NO: 5. The amino acid sequence of an exemplary mature human ACE2 protein is set forth in SEQ ID NO: 78.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

As used herein, the term “SARS-CoV-2 spike” protein refers to the SARS-CoV-2 protein that mediates binding to the host cell receptor angiotensin-converting enzyme 2 (ACE2). An exemplary amino acid sequence of a reference immature SARS-CoV-2 spike protein is set forth in SEQ ID NO: 1. The term SARS-CoV-2 spike protein includes naturally occurring and engineered variants.

As used herein, the term “set” with reference to amino acid variation(s) (e.g., substitution(s)) does not require more than one amino acid variation (e.g., substitution). For example, Table 2 and Table 4 herein describe “sets” of amino acid substitutions, some sets have only one amino acid substitution and some sets have more than one amino acid substitution.

As used herein, the term “signal peptide” or “signal sequence” refers to a sequence (e.g., an amino acid sequence) that can direct the transport or localization of a protein to a certain organelle, cell compartment, or extracellular export. The term encompasses both the signal peptide (the amino acid sequence of the signal peptide) and the nucleic acid sequence encoding the signal peptide. Thus, references to a signal peptide in the context of a nucleic acid molecule refers to the nucleic acid sequence encoding the signal peptide.

As used herein, the term “subject” includes any animal, such as a human or other animal. In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

A “therapeutically effective amount” of an agent (e.g., a therapeutic agent, a vaccine) refers to any amount of the agent (e.g., the therapeutic agent, the vaccine) that, when used alone or in combination with another agent (e.g., a therapeutic agent, a vaccine), protects a subject against the onset of a disease (e.g., an infection), ameliorates the severity of a disease (e.g., an infection), and/or promotes disease (e.g., infection) regression evidenced by a decrease in severity of disease (e.g., infection) symptoms, an increase in frequency and duration of disease (e.g., infection) symptom-free periods, or a prevention of impairment or disability due to the disease (e.g., infection) affliction. The ability of an agent (e.g., a therapeutic agent, a vaccine) to do any of the foregoing (or any combination thereof) can be evaluated using a variety of methods known to the skilled practitioner, such as in human subjects during clinical trials, in animal model systems predictive of efficacy in humans, or by assaying the activity of the agent in in vitro assays.

As used herein, the term “translatable RNA” refers to any RNA that encodes at least one polypeptide or protein and can be translated to produce the encoded polypeptide or protein in vitro, in vivo, in situ or ex vivo. A translatable RNA may be an mRNA or a circular RNA encoding a polypeptide or protein.

As used herein, the terms “treat,” treating,” “treatment,” and the like refer to reducing or ameliorating a disease or infection and/or symptom(s) associated therewith or obtaining a desired pharmacologic and/or physiologic effect. It will be appreciated that, although not precluded, treating a disease or infection does not require that the disease or infection, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease or infection. In some embodiments, the effect is preventative, i.e., the effect protects or prevents an occurrence or reoccurrence of a disease or infection. To this end, the presently disclosed methods comprise administering a therapeutically effective amount of a compositions as described herein.

As used herein, the term “variant” or “variation” with reference to a nucleic acid molecule, refers to a nucleic acid molecule that comprises at least one substitution, alteration, inversion, addition, or deletion of nucleotide compared to a reference nucleic acid molecule. As used herein, the term “variant” or “variation” with reference to a peptide or protein refers to a peptide or protein that comprises at least one substitution, alteration, inversion, addition, or deletion of an amino acid residue compared to a reference peptide or protein.

As used herein, the term “5′-untranslated region” or “5′-UTR” refers to a part of a nucleic acid molecule located 5′ (i.e., “upstream”) of a coding sequence and which is not translated into protein. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g., by enzymatic or post-transcriptional addition of a 5′-cap structure.

As used herein the term “3′-untranslated region” or “3′-UTR” refers to a part of a nucleic acid molecule located 3′ (i.e., downstream) of a coding sequence and which is not translated into protein. A 3′-UTR may located between a coding sequence and an (optional) terminal poly(A) sequence of a nucleic acid sequence. A 3′-UTR may comprise elements for controlling gene expression, also called regulatory elements. Such regulatory elements may be, e.g., ribosomal binding sites, miRNA binding sites etc.

### 5.2 SARS-CoV-2 Spike Proteins (e.g., Immunogens)

The SARS-CoV-2 spike protein mediates viral entry into host cells. The spike protein comprises two functional subunits responsible for binding to the host cell receptor (S1 subunit) and fusion of the viral and cellular membranes (S2 subunit). The SARS-CoV-2 spike protein is cleaved at the boundary between the S1 and S2 subunits, which remain non-covalently associated in the prefusion conformation. The distal S1 subunit comprises the RBD and contributes to stabilization of the prefusion state of the membrane anchored S2 subunit that contains the fusion machinery. The RBD mediates binding to the host cell receptor ACE2. The spike protein is cleaved by host proteases at the so-called S2′ site located immediately upstream of the fusion peptide. This cleavage has been proposed to activate the protein for membrane fusion via extensive irreversible conformational changes. (See, e.g., Walls, Alexandra C et al. “Structure, Function, and Antigenicity of the SARS-CoV-2 Spike Glycoprotein.” Cell vol. 181, 2 (2020): 281-292.e6. doi:10.1016/j.cell.2020.02.058, the entire contents of which is incorporated herein by reference for all purposes).

The amino acid sequence of a first reference immature SARS-CoV-2 spike protein is provided in SEQ ID NO: 1. The signal peptide is underlined (amino acids 1-13 of SEQ ID NO: 1); the amino acid sequence of the corresponding mature first reference SARS-CoV-2 spike protein lacking the native signal peptide is provided in SEQ ID NO: 2. The amino acid sequence of an exemplary RBD of the first reference SARS-CoV-2 spike protein is set forth in SEQ ID NO: 5.

The amino acid sequence of a second reference immature SARS-CoV-2 spike protein is provided in SEQ ID NO: 3. The signal peptide is underlined (amino acids 1-13 of SEQ ID NO: 3); the amino acid sequence of the corresponding mature second reference SARS-CoV-2 spike protein lacking the native signal peptide is provided in SEQ ID NO: 4. The amino acid sequence of the second reference SARS-CoV-2 spike protein comprises a proline at amino acid positions 986 and 987, amino acid numbering relative to SEQ ID NO: 1. This diproline amino acid substitution stabilizes said SARS-CoV-2 spike protein in a prefusion state. The amino acid sequence of an exemplary RBD of the second reference SARS-CoV-2 spike protein is set forth in SEQ ID NO: 5.

The amino acid sequence of a third reference immature SARS-CoV-2 spike protein is provided in SEQ ID NO: 6. The signal peptide is underlined (amino acids 1-13 of SEQ ID NO: 6); the amino acid sequence of the corresponding mature third reference SARS-CoV-2 spike protein lacking the native signal peptide is provided in SEQ ID NO: 7. The amino acid sequence of an exemplary RBD of the third reference SARS-CoV-2 spike protein is set forth in SEQ ID NO: 10.

The amino acid sequence of a fourth reference immature SARS-CoV-2 spike protein is provided in SEQ ID NO: 8. The signal peptide is underlined (amino acids 1-13 of SEQ ID NO: 8); the amino acid sequence of the corresponding mature fourth reference SARS-CoV-2 spike protein lacking the native signal peptide is provided in SEQ ID NO: 9. The fourth reference SARS-CoV-2 spike protein comprises a proline at amino acid positions 986 and 987, amino acid numbering relative to SEQ ID NO: 1. This diproline amino acid substitution stabilizes said SARS-CoV-2 spike protein in a prefusion state. The amino acid sequence of an exemplary RBD of the fourth reference SARS-CoV-2 spike protein is set forth in SEQ ID NO: 10.

The amino acid sequence of an exemplary reference human ACE2 (hACE2) proteins is also set forth in SEQ ID NO: 77 (immature) and SEQ ID NO: 78 (mature).

TABLE 1
Amino Acid Sequence of Reference SARS-CoV-2 Spike Proteins
(and Exemplary RBDs Thereof) and hACE2

| | | SEQ |
|---|---|---|
| Description | Amino Acid Sequence | ID NO |
| Immature SARS-CoV-2 | MFVFLVLLPLVSSQCVNLTTRTQLPPAYTNSFTRGVYYP | 1 |
| Spike Protein Reference 1 | DKVFRSSVLHSTQDLFLPFFSNVTWFHAIHVSGTNGTKR | |
| | FDNPVLPENDGVYFASTEKSNIIRGWIFGTTLDSKTQSL | |
| | LIVNNATNVVIKVCEFQFCNDPFLGVYYHKNNKSWMESE | |
| | FRVYSSANNCTFEYVSQPFLMDLEGKQGNEKNLREFVEK | |
| | NIDGYFKIYSKHTPINLVRDLPQGFSALEPLVDLPIGIN | |
| | ITRFQTLLALHRSYLTPGDSSSGWTAGAAAYYVGYLQPR | |
| | TELLKYNENGTITDAVDCALDPLSETKCTLKSFTVEKGI | |
| | YQTSNERVQPTESIVRFPNITNLCPFGEVFNATRFASVY | |
| | AWNRKRISNCVADYSVLYNSASFSTFKCYGVSPTKLNDL | |
| | CFTNVYADSFVIRGDEVRQIAPGQTGKIADYNYKLPDDE | |
| | TGCVIAWNSNNLDSKVGGNYNYLYRLFRKSNLKPFERDI | |
| | STEIYQAGSTPCNGVEGENCYFPLQSYGFQPTNGVGYQP | |
| | YRVVVLSFELLHAPATVCGPKKSTNLVKNKCVNENENGL | |
| | TGTGVLTESNKKELPFQQFGRDIADTTDAVRDPQTLEIL | |
| | DITPCSFGGVSVITPGTNTSNQVAVLYQDVNCTEVPVAI | |
| | HADQLTPTWRVYSTGSNVFQTRAGCLIGAEHVNNSYECD | |
| | IPIGAGICASYQTQTNSPRRARSVASQSIIAYTMSLGAE | |
| | NSVAYSNNSIAIPTNFTISVTTEILPVSMTKTSVDCTMY | |
| | ICGDSTECSNLLLQYGSFCTQLNRALTGIAVEQDKNTQE | |
| | VFAQVKQIYKTPPIKDFGGENFSQILPDPSKPSKRSFIE | |
| | DLLFNKVTLADAGFIKQYGDCLGDIAARDLICAQKENGL | |
| | TVLPPLLTDEMIAQYTSALLAGTITSGWTFGAGAALQIP | |
| | FAMQMAYRENGIGVTQNVLYENQKLIANQFNSAIGKIQD | |
| | SLSSTASALGKLQDVVNQNAQALNTLVKQLSSNEGAISS | |
| | VLNDILSRLDKVEAEVOIDRLITGRLQSLQTYVTQQLIR | |
| | AAEIRASANLAATKMSECVLGQSKRVDFCGKGYHLMSFP | |
| | QSAPHGVVFLHVTYVPAQEKNFTTAPAICHDGKAHFPRE | |
| | GVFVSNGTHWFVTQRNFYEPQIITTDNTFVSGNCDVVIG | |
| | IVNNTVYDPLQPELDSFKEELDKYFKNHTSPDVDLGDIS | |
| | GINASVVNIQKEIDRLNEVAKNLNESLIDLQELGKYEQY | |
| | IKWPWYIWLGFIAGLIAIVMVTIMLCCMTSCCSCLKGCC | |
| | SCGSCCKFDEDDSEPVLKGVKLHYT | |
| Mature SARS-CoV-2 Spike | QCVNLTTRTQLPPAYTNSFTRGVYYPDKVERSSVLHSTQ | 2 |
| Protein Reference 1 | DLFLPFFSNVTWFHAIHVSGTNGTKREDNPVLPENDGVY | |
| Lacking native signal | FASTEKSNIIRGWIFGTTLDSKTQSLLIVNNATNVVIKV | |
| peptide | CEFQFCNDPFLGVYYHKNNKSWMESEFRVYSSANNCTFE | |
| | YVSQPFLMDLEGKQGNEKNLREFVEKNIDGYFKIYSKHT | |
| | PINLVRDLPQGFSALEPLVDLPIGINITRFQTLLALHRS | |
| | YLTPGDSSSGWTAGAAAYYVGYLQPRTELLKYNENGTIT | |
| | DAVDCALDPLSETKCTLKSFTVEKGIYQTSNERVOPTES | |
| | IVRFPNITNLCPFGEVFNATRFASVYAWNRKRISNCVAD | |
| | YSVLYNSASFSTFKCYGVSPTKLNDLCFTNVYADSEVIR | |
| | GDEVRQIAPGQTGKIADYNYKLPDDFTGCVIAWNSNNLD | |
| | SKVGGNYNYLYRLERKSNLKPFERDISTEIYQAGSTPCN | |
| | GVEGFNCYFPLQSYGFQPTNGVGYQPYRVVVLSFELLHA | |
| | PATVCGPKKSTNLVKNKCVNFNENGLTGTGVLTESNKKF | |
| | LPFQQFGRDIADTTDAVRDPQTLEILDITPCSFGGVSVI | |
| | TPGTNTSNQVAVLYQDVNCTEVPVAIHADQLTPTWRVYS | |
| | TGSNVFQTRAGCLIGAEHVNNSYECDIPIGAGICASYQT | |
| | QTNSPRRARSVASQSIIAYTMSLGAENSVAYSNNSIAIP | |
| | TNFTISVTTEILPVSMTKTSVDCTMYICGDSTECSNLLL | |
| | QYGSFCTQLNRALTGIAVEQDKNTQEVFAQVKQIYKTPP | |
| | IKDFGGFNFSQILPDPSKPSKRSFIEDLLENKVTLADAG | |
| | FIKQYGDCLGDIAARDLICAQKENGLTVLPPLLTDEMIA | |
| | QYTSALLAGTITSGWTFGAGAALQIPFAMQMAYRENGIG | |
| | VTQNVLYENQKLIANQFNSAIGKIQDSLSSTASALGKLQ | |
| | DVVNQNAQALNTLVKQLSSNFGAISSVLNDILSRLDKVE | |
| | AEVQIDRLITGRLQSLQTYVTQQLIRAAEIRASANLAAT | |
| | KMSECVLGQSKRVDFCGKGYHLMSFPQSAPHGVVELHVT | |
| | YVPAQEKNFTTAPAICHDGKAHFPREGVFVSNGTHWEVT | |
| | QRNFYEPQIITTDNTFVSGNCDVVIGIVNNTVYDPLQPE | |
| | LDSFKEELDKYFKNHTSPDVDLGDISGINASVVNIQKEI | |
| | DRLNEVAKNLNESLIDLQELGKYEQYIKWPWYIWLGFIA | |
| | GLIAIVMVTIMLCCMTSCCSCLKGCCSCGSCCKEDEDDS | |
| | EPVLKGVKLHYT | |
| SARS-CoV-2 RBD of | NITNLCPFGEVFNATRFASVYAWNRKRISNCVADYSVLY | 5 |
| Spike Protein Reference 1 | NSASFSTFKCYGVSPTKLNDLCFTNVYADSFVIRGDEVR | |
| | QIAPGQTGKIADYNYKLPDDFTGCVIAWNSNNLDSKVGG | |
| | NYNYLYRLERKSNLKPFERDISTEIYQAGSTPCNGVEGF | |
| | NCYFPLQSYGFQPTNGVGYQPYRVVVLSFELLHAPATVC | |
| | GPKKST | |
| Immature SARS-CoV-2 | MFVFLVLLPLVSSQCVNLTTRTQLPPAYTNSFTRGVYYP | 3 |
| Spike Protein Reference 2 | DKVFRSSVLHSTQDLFLPFFSNVTWFHAIHVSGTNGTKR | |
| | FDNPVLPENDGVYFASTEKSNIIRGWIFGTTLDSKTQSL | |
| | LIVNNATNVVIKVCEFQFCNDPFLGVYYHKNNKSWMESE | |
| | FRVYSSANNCTFEYVSQPFLMDLEGKQGNEKNLREFVEK | |
| | NIDGYFKIYSKHTPINLVRDLPQGFSALEPLVDLPIGIN | |
| | ITRFQTLLALHRSYLTPGDSSSGWTAGAAAYYVGYLQPR | |
| | TELLKYNENGTITDAVDCALDPLSETKCTLKSFTVEKGI | |
| | YQTSNERVQPTESIVRFPNITNLCPFGEVFNATRFASVY | |
| | AWNRKRISNCVADYSVLYNSASFSTFKCYGVSPTKLNDL | |
| | CFTNVYADSFVIRGDEVRQIAPGQTGKIADYNYKLPDDE | |
| | TGCVIAWNSNNLDSKVGGNYNYLYRLERKSNLKPFERDI | |
| | STEIYQAGSTPCNGVEGENCYFPLQSYGFQPTNGVGYQP | |
| | YRVVVLSFELLHAPATVCGPKKSTNLVKNKCVNENENGL | |
| | TGTGVLTESNKKELPFQQFGRDIADTTDAVRDPQTLEIL | |
| | DITPCSFGGVSVITPGTNTSNQVAVLYQDVNCTEVPVAI | |
| | HADQLTPTWRVYSTGSNVFQTRAGCLIGAEHVNNSYECD | |
| | IPIGAGICASYQTQTNSPRRARSVASQSIIAYTMSLGAE | |
| | NSVAYSNNSIAIPTNFTISVTTEILPVSMTKTSVDCTMY | |
| | ICGDSTECSNLLLQYGSFCTQLNRALTGIAVEQDKNTQE | |
| | VFAQVKQIYKTPPIKDFGGENFSQILPDPSKPSKRSFIE | |
| | DLLFNKVTLADAGFIKQYGDCLGDIAARDLICAQKENGL | |
| | TVLPPLLTDEMIAQYTSALLAGTITSGWTFGAGAALQIP | |
| | FAMQMAYRENGIGVTQNVLYENQKLIANQFNSAIGKIQD | |
| | SLSSTASALGKLQDVVNQNAQALNTLVKQLSSNEGAISS | |
| | VLNDILSRLDPPEAEVQIDRLITGRLQSLQTYVTQQLIR | |
| | AAEIRASANLAATKMSECVLGQSKRVDFCGKGYHLMSFP | |
| | QSAPHGVVFLHVTYVPAQEKNFTTAPAICHDGKAHFPRE | |
| | GVFVSNGTHWFVTQRNFYEPQIITTDNTFVSGNCDVVIG | |
| | IVNNTVYDPLQPELDSFKEELDKYFKNHTSPDVDLGDIS | |
| | GINASVVNIQKEIDRLNEVAKNLNESLIDLQELGKYEQY | |
| | IKWPWYIWLGFIAGLIAIVMVTIMLCCMTSCCSCLKGCC | |
| | SCGSCCKFDEDDSEPVLKGVKLHYT | |
| Mature SARS-CoV-2 Spike | QCVNLTTRTQLPPAYTNSFTRGVYYPDKVERSSVLHSTQ | 4 |
| Protein Reference 2 | DLFLPFFSNVTWFHAIHVSGTNGTKRFDNPVLPENDGVY | |
| Lacking native signal | FASTEKSNIIRGWIFGTTLDSKTQSLLIVNNATNVVIKV | |
| peptide | CEFQFCNDPFLGVYYHKNNKSWMESEFRVYSSANNCTFE | |
| | YVSQPFLMDLEGKQGNEKNLREFVEKNIDGYFKIYSKHT | |
| | PINLVRDLPQGFSALEPLVDLPIGINITRFQTLLALHRS | |
| | YLTPGDSSSGWTAGAAAYYVGYLQPRTELLKYNENGTIT | |
| | DAVDCALDPLSETKCTLKSFTVEKGIYQTSNERVOPTES | |
| | IVRFPNITNLCPFGEVFNATRFASVYAWNRKRISNCVAD | |
| | YSVLYNSASFSTFKCYGVSPTKLNDLCFTNVYADSEVIR | |
| | GDEVRQIAPGQTGKIADYNYKLPDDFTGCVIAWNSNNLD | |
| | SKVGGNYNYLYRLERKSNLKPFERDISTEIYQAGSTPCN | |
| | GVEGFNCYFPLQSYGFQPTNGVGYQPYRVVVLSFELLHA | |
| | PATVCGPKKSTNLVKNKCVNFNENGLTGTGVLTESNKKE | |
| | LPFQQFGRDIADTTDAVRDPQTLEILDITPCSFGGVSVI | |
| | TPGTNTSNQVAVLYQDVNCTEVPVAIHADQLTPTWRVYS | |
| | TGSNVFQTRAGCLIGAEHVNNSYECDIPIGAGICASYQT | |
| | QTNSPRRARSVASQSIIAYTMSLGAENSVAYSNNSIAIP | |
| | TNFTISVTTEILPVSMTKTSVDCTMYICGDSTECSNLLL | |
| | QYGSFCTQLNRALTGIAVEQDKNTQEVFAQVKQIYKTPP | |
| | IKDFGGFNFSQILPDPSKPSKRSFIEDLLENKVTLADAG | |
| | FIKQYGDCLGDIAARDLICAQKENGLTVLPPLLTDEMIA | |
| | QYTSALLAGTITSGWTFGAGAALQIPFAMQMAYRENGIG | |
| | VTQNVLYENQKLIANQFNSAIGKIQDSLSSTASALGKLQ | |
| | DVVNQNAQALNTLVKQLSSNFGAISSVLNDILSRLDPPE | |
| | AEVQIDRLITGRLQSLQTYVTQQLIRAAEIRASANLAAT | |
| | KMSECVLGQSKRVDFCGKGYHLMSFPQSAPHGVVELHVT | |
| | YVPAQEKNFTTAPAICHDGKAHFPREGVFVSNGTHWFVT | |
| | QRNFYEPQIITTDNTFVSGNCDVVIGIVNNTVYDPLQPE | |
| | LDSFKEELDKYFKNHTSPDVDLGDISGINASVVNIQKEI | |
| | DRLNEVAKNLNESLIDLQELGKYEQYIKWPWYIWLGFIA | |
| | GLIAIVMVTIMLCCMTSCCSCLKGCCSCGSCCKEDEDDS | |
| | EPVLKGVKLHYT | |
| SARS-CoV-2 RBD of | NITNLCPFGEVFNATRFASVYAWNRKRISNCVADYSVLY | 5 |
| Spike Protein Reference 2 | NSASFSTFKCYGVSPTKLNDLCFTNVYADSFVIRGDEVR | |
| | QIAPGQTGKIADYNYKLPDDFTGCVIAWNSNNLDSKVGG | |
| | NYNYLYRLFRKSNLKPFERDISTEIYQAGSTPCNGVEGF | |
| | NCYFPLQSYGFQPTNGVGYQPYRVVVLSFELLHAPATVC | |
| | GPKKST | |
| Immature SARS-CoV-2 | MFVFLVLLPLVSSQCVNLITRTQSYTNSFTRGVYYPDKV | 6 |
| Spike Protein Reference 3 | FRSSVLHSTQDLFLPFFSNVTWFHAISGTNGTKREDNPV | |
| | LPFNDGVYFASTEKSNIIRGWIFGTTLDSKTQSLLIVNN | |
| | ATNVVIKVCEFQFCNDPFLDVYYHKNNKSWMESEFRVYS | |
| | SANNCTFEYVSQPFLMDLEGKQGNEKNLREFVEKNIDGY | |
| | FKIYSKHTPINLGRDLPQGFSALEPLVDLPIGINITRFQ | |
| | TLLALHRSYLTPGDSSSGWTAGAAAYYVGYLQPRTELLK | |
| | YNENGTITDAVDCALDPLSETKCTLKSFTVEKGIYQTSN | |
| | FRVQPTESIVRFPNITNLCPFDEVFNATRFASVYAWNRK | |
| | RISNCVADYSVLYNFAPFFAFKCYGVSPTKLNDLCFTNV | |
| | YADSFVIRGNEVSQIAPGQTGNIADYNYKLPDDFTGCVI | |
| | AWNSNKLDSKVGGNYNYRYRLFRKSNLKPFERDISTEIY | |
| | QAGNKPCNGVAGVNCYFPLQSYGFRPTYGVGHQPYRVVV | |
| | LSFELLHAPATVCGPKKSTNLVKNKCVNENENGLTGTGV | |
| | LTESNKKELPFQQFGRDIADTTDAVRDPQTLEILDITPC | |
| | SFGGVSVITPGTNTSNQVAVLYQGVNCTEVPVAIHADQL | |
| | TPTWRVYSTGSNVFQTRAGCLIGAEYVNNSYECDIPIGA | |
| | GICASYQTQTKSHRRARSVASQSIIAYTMSLGAENSVAY | |
| | SNNSIAIPTNFTISVTTEILPVSMTKTSVDCTMYICGDS | |
| | TECSNLLLQYGSFCTQLKRALTGIAVEQDKNTQEVFAQV | |
| | KQIYKTPPIKYFGGFNFSQILPDPSKPSKRSFIEDLLEN | |
| | KVTLADAGFIKQYGDCLGDIAARDLICAQKENGLTVLPP | |
| | LLTDEMIAQYTSALLAGTITSGWTFGAGAALQIPFAMQM | |
| | AYRENGIGVTQNVLYENQKLIANQFNSAIGKIQDSLSST | |
| | ASALGKLQDVVNHNAQALNTLVKQLSSKFGAISSVLNDI | |
| | LSRLDKVEAEVOIDRLITGRLQSLQTYVTQQLIRAAEIR | |
| | ASANLAATKMSECVLGQSKRVDFCGKGYHLMSFPQSAPH | |
| | GVVFLHVTYVPAQEKNFTTAPAICHDGKAHFPREGVEVS | |
| | NGTHWFVTQRNFYEPQIITTDNTFVSGNCDVVIGIVNNT | |
| | VYDPLQPELDSFKEELDKYFKNHTSPDVDLGDISGINAS | |
| | VVNIQKEIDRLNEVAKNLNESLIDLQELGKYEQYIKWPW | |
| | YIWLGFIAGLIAIVMVTIMLCCMTSCCSCLKGCCSCGSC | |
| | CKFDEDDSEPVLKGVKLHYT | |
| Mature SARS-CoV-2 Spike | QCVNLITRTQSYTNSFTRGVYYPDKVERSSVLHSTQDLF | 7 |
| Protein Reference 3 | LPFFSNVTWFHAISGTNGTKRFDNPVLPENDGVYFASTE | |
| Lacking native signal | KSNIIRGWIFGTTLDSKTQSLLIVNNATNVVIKVCEFQF | |
| peptide | CNDPFLDVYYHKNNKSWMESEFRVYSSANNCTFEYVSQP | |
| | FLMDLEGKQGNFKNLREFVEKNIDGYFKIYSKHTPINLG | |
| | RDLPQGFSALEPLVDLPIGINITRFQTLLALHRSYLTPG | |
| | DSSSGWTAGAAAYYVGYLQPRTELLKYNENGTITDAVDC | |
| | ALDPLSETKCTLKSFTVEKGIYQTSNERVQPTESIVREP | |
| | NITNLCPFDEVFNATRFASVYAWNRKRISNCVADYSVLY | |
| | NFAPFFAFKCYGVSPTKLNDLCFTNVYADSFVIRGNEVS | |
| | QIAPGQTGNIADYNYKLPDDFTGCVIAWNSNKLDSKVGG | |
| | NYNYRYRLFRKSNLKPFERDISTEIYQAGNKPCNGVAGV | |
| | NCYFPLQSYGFRPTYGVGHQPYRVVVLSFELLHAPATVC | |
| | GPKKSTNLVKNKCVNFNFNGLTGTGVLTESNKKELPFQQ | |
| | FGRDIADTTDAVRDPQTLEILDITPCSFGGVSVITPGTN | |
| | TSNQVAVLYQGVNCTEVPVAIHADQLTPTWRVYSTGSNV | |
| | FQTRAGCLIGAEYVNNSYECDIPIGAGICASYQTQTKSH | |
| | RRARSVASQSIIAYTMSLGAENSVAYSNNSIAIPTNFTI | |
| | SVTTEILPVSMTKTSVDCTMYICGDSTECSNLLLQYGSF | |
| | CTQLKRALTGIAVEQDKNTQEVFAQVKQIYKTPPIKYFG | |
| | GFNFSQILPDPSKPSKRSFIEDLLENKVTLADAGFIKQY | |
| | GDCLGDIAARDLICAQKENGLTVLPPLLTDEMIAQYTSA | |
| | LLAGTITSGWTFGAGAALQIPFAMQMAYRENGIGVTQNV | |
| | LYENQKLIANQFNSAIGKIQDSLSSTASALGKLQDVVNH | |
| | NAQALNTLVKQLSSKFGAISSVLNDILSRLDKVEAEVQI | |
| | DRLITGRLQSLQTYVTQQLIRAAEIRASANLAATKMSEC | |
| | VLGQSKRVDFCGKGYHLMSFPQSAPHGVVFLHVTYVPAQ | |
| | EKNFTTAPAICHDGKAHFPREGVFVSNGTHWFVTORNFY | |
| | EPQIITTDNTFVSGNCDVVIGIVNNTVYDPLQPELDSEK | |
| | EELDKYFKNHTSPDVDLGDISGINASVVNIQKEIDRLNE | |
| | VAKNLNESLIDLQELGKYEQYIKWPWYIWLGFIAGLIAI | |
| | VMVTIMLCCMTSCCSCLKGCCSCGSCCKFDEDDSEPVLK | |
| | GVKLHYT | |
| SARS-CoV-2 RBD of | NITNLCPFDEVFNATRFASVYAWNRKRISNCVADYSVLY | 10 |
| Spike Protein Reference 3 | NEAPFFAFKCYGVSPTKLNDLCFTNVYADSFVIRGNEVS | |
| | QIAPGQTGNIADYNYKLPDDFTGCVIAWNSNKLDSKVGG | |
| | NYNYRYRLERKSNLKPFERDISTEIYQAGNKPCNGVAGV | |
| | NCYFPLQSYGFRPTYGVGHQPYRVVVLSFELLHAPATVC | |
| | GPKKST | |
| Immature SARS-CoV-2 | MFVFLVLLPLVSSQCVNLITRTQSYTNSFTRGVYYPDKV | 8 |
| Spike Protein Reference 4 | FRSSVLHSTQDLFLPFFSNVTWFHAISGTNGTKRFDNPV | |
| | LPFNDGVYFASTEKSNIIRGWIFGTTLDSKTQSLLIVNN | |
| | ATNVVIKVCEFQFCNDPFLDVYYHKNNKSWMESEFRVYS | |
| | SANNCTFEYVSQPFLMDLEGKQGNEKNLREFVEKNIDGY | |
| | FKIYSKHTPINLGRDLPQGFSALEPLVDLPIGINITRFQ | |
| | TLLALHRSYLTPGDSSSGWTAGAAAYYVGYLQPRTELLK | |
| | YNENGTITDAVDCALDPLSETKCTLKSFTVEKGIYQTSN | |
| | FRVQPTESIVRFPNITNLCPFDEVFNATRFASVYAWNRK | |
| | RISNCVADYSVLYNFAPFFAFKCYGVSPTKLNDLCFTNV | |
| | YADSFVIRGNEVSQIAPGQTGNIADYNYKLPDDFTGCVI | |
| | AWNSNKLDSKVGGNYNYRYRLERKSNLKPFERDISTEIY | |
| | QAGNKPCNGVAGVNCYFPLQSYGFRPTYGVGHQPYRVVV | |
| | LSFELLHAPATVCGPKKSTNLVKNKCVNENENGLTGTGV | |
| | LTESNKKELPFQQFGRDIADTTDAVRDPQTLEILDITPC | |
| | SFGGVSVITPGTNTSNOVAVLYQGVNCTEVPVAIHADQL | |
| | TPTWRVYSTGSNVFQTRAGCLIGAEYVNNSYECDIPIGA | |
| | GICASYQTQTKSHRRARSVASQSIIAYTMSLGAENSVAY | |
| | SNNSIAIPTNFTISVTTEILPVSMTKTSVDCTMYICGDS | |
| | TECSNLLLQYGSFCTQLKRALTGIAVEQDKNTQEVFAQV | |
| | KQIYKTPPIKYFGGENFSQILPDPSKPSKRSFIEDLLEN | |
| | KVTLADAGFIKQYGDCLGDIAARDLICAQKENGLTVLPP | |
| | LLTDEMIAQYTSALLAGTITSGWTFGAGAALQIPFAMQM | |
| | AYRENGIGVTQNVLYENQKLIANQFNSAIGKIQDSLSST | |
| | ASALGKLQDVVNHNAQALNTLVKQLSSKFGAISSVLNDI | |
| | LSRLDPPEAEVQIDRLITGRLQSLQTYVTQQLIRAAEIR | |
| | ASANLAATKMSECVLGQSKRVDFCGKGYHLMSFPQSAPH | |
| | GVVFLHVTYVPAQEKNFTTAPAICHDGKAHFPREGVEVS | |
| | NGTHWFVTQRNFYEPQIITTDNTFVSGNCDVVIGIVNNT | |
| | VYDPLQPELDSFKEELDKYFKNHTSPDVDLGDISGINAS | |
| | VVNIQKEIDRLNEVAKNLNESLIDLQELGKYEQYIKWPW | |
| | YIWLGFIAGLIAIVMVTIMLCCMTSCCSCLKGCCSCGSC | |
| | CKFDEDDSEPVLKGVKLHYT | |
| Mature SARS-CoV-2 Spike | QCVNLITRTQSYTNSFTRGVYYPDKVERSSVLHSTQDLE | 9 |
| Protein Reference 4 | LPFFSNVTWFHAISGTNGTKREDNPVLPENDGVYFASTE | |
| Lacking native signal | KSNIIRGWIFGTTLDSKTQSLLIVNNATNVVIKVCEFQF | |
| peptide | CNDPELDVYYHKNNKSWMESEFRVYSSANNCTFEYVSQP | |
| | FLMDLEGKQGNEKNLREFVEKNIDGYFKIYSKHTPINLG | |
| | RDLPQGFSALEPLVDLPIGINITRFQTLLALHRSYLTPG | |
| | DSSSGWTAGAAAYYVGYLQPRTELLKYNENGTITDAVDC | |
| | ALDPLSETKCTLKSFTVEKGIYQTSNERVQPTESIVREP | |
| | NITNLCPFDEVFNATRFASVYAWNRKRISNCVADYSVLY | |
| | NEAPFFAFKCYGVSPTKLNDLCFTNVYADSEVIRGNEVS | |
| | QIAPGQTGNIADYNYKLPDDFTGCVIAWNSNKLDSKVGG | |
| | NYNYRYRLFRKSNLKPFERDISTEIYQAGNKPCNGVAGV | |
| | NCYFPLQSYGFRPTYGVGHQPYRVVVLSFELLHAPATVC | |
| | GPKKSTNLVKNKCVNFNFNGLTGTGVLTESNKKELPFQQ | |
| | FGRDIADTTDAVRDPQTLEILDITPCSFGGVSVITPGTN | |
| | TSNQVAVLYQGVNCTEVPVAIHADQLTPTWRVYSTGSNV | |
| | FQTRAGCLIGAEYVNNSYECDIPIGAGICASYQTQTKSH | |
| | RRARSVASQSIIAYTMSLGAENSVAYSNNSIAIPTNFTI | |
| | SVTTEILPVSMTKTSVDCTMYICGDSTECSNLLLQYGSF | |
| | CTQLKRALTGIAVEQDKNTQEVFAQVKQIYKTPPIKYFG | |
| | GFNFSQILPDPSKPSKRSFIEDLLENKVTLADAGFIKQY | |
| | GDCLGDIAARDLICAQKENGLTVLPPLLTDEMIAQYTSA | |
| | LLAGTITSGWTFGAGAALQIPFAMQMAYRENGIGVTQNV | |
| | LYENQKLIANQFNSAIGKIQDSLSSTASALGKLQDVVNH | |
| | NAQALNTLVKQLSSKFGAISSVLNDILSRLDPPEAEVQI | |
| | DRLITGRLQSLQTYVTQQLIRAAEIRASANLAATKMSEC | |
| | VLGQSKRVDFCGKGYHLMSFPQSAPHGVVFLHVTYVPAQ | |
| | EKNFTTAPAICHDGKAHFPREGVFVSNGTHWFVTORNFY | |
| | EPQIITTDNTFVSGNCDVVIGIVNNTVYDPLQPELDSEK | |
| | EELDKYFKNHTSPDVDLGDISGINASVVNIQKEIDRLNE | |
| | VAKNLNESLIDLQELGKYEQYIKWPWYIWLGFIAGLIAI | |
| | VMVTIMLCCMTSCCSCLKGCCSCGSCCKFDEDDSEPVLK | |
| | GVKLHYT | |
| SARS-CoV-2 RBD of | NITNLCPFDEVFNATRFASVYAWNRKRISNCVADYSVLY | 10 |
| Spike Protein Reference 4 | NFAPFFAFKCYGVSPTKLNDLCFTNVYADSFVIRGNEVS | |
| | QIAPGQTGNIADYNYKLPDDFTGCVIAWNSNKLDSKVGG | |
| | NYNYRYRLERKSNLKPFERDISTEIYQAGNKPCNGVAGV | |
| | NCYFPLQSYGFRPTYGVGHQPYRVVVLSFELLHAPATVC | |
| | GPKKST | |
| Immature hACE2 | MSSSSWLLLSLVAVTAAQSTIEEQAKTELDKENHEAEDL | 77 |
| Signal Sequence | FYQSSLASWNYNTNITEENVQNMNNAGDKWSAFLKEQST | |
| Underlined | LAQMYPLQEIQNLTVKLQLQALQQNGSSVLSEDKSKRLN | |
| | TILNTMSTIYSTGKVCNPDNPQECLLLEPGLNEIMANSL | |
| | DYNERLWAWESWRSEVGKQLRPLYEEYVVLKNEMARANH | |
| | YEDYGDYWRGDYEVNGVDGYDYSRGQLIEDVEHTFEEIK | |
| | PLYEHLHAYVRAKLMNAYPSYISPIGCLPAHLLGDMWGR | |
| | FWTNLYSLTVPFGQKPNIDVTDAMVDQAWDAQRIFKEAE | |
| | KFFVSVGLPNMTQGFWENSMLTDPGNVQKAVCHPTAWDL | |
| | GKGDFRILMCTKVTMDDELTAHHEMGHIQYDMAYAAQPF | |
| | LLRNGANEGFHEAVGEIMSLSAATPKHLKSIGLLSPDFQ | |
| | EDNETEINFLLKQALTIVGTLPFTYMLEKWRWMVEKGEI | |
| | PKDQWMKKWWEMKREIVGVVEPVPHDETYCDPASLFHVS | |
| | NDYSFIRYYTRTLYQFQFQEALCQAAKHEGPLHKCDISN | |
| | STEAGQKLENMLRLGKSEPWTLALENVVGAKNMNVRPLL | |
| | NYFEPLFTWLKDONKNSFVGWSTDWSPYADQSIKVRISL | |
| | KSALGDKAYEWNDNEMYLERSSVAYAMRQYFLKVKNQMI | |
| | LFGEEDVRVANLKPRISENFFVTAPKNVSDIIPRTEVEK | |
| | AIRMSRSRINDAFRLNDNSLEFLGIQPTLGPPNQPPVSI | |
| | WLIVFGVVMGVIVVGIVILIFTGIRDRKKKNKARSGENP | |
| | YASIDISKGENNPGFQNTDDVQTSF | |
| Mature hACE2 | QSTIEEQAKTFLDKENHEAEDLFYQSSLASWNYNTNITE | 78 |
| No Signal Sequence | ENVQNMNNAGDKWSAFLKEQSTLAQMYPLQEIQNLTVKL | |
| | QLQALQQNGSSVLSEDKSKRLNTILNTMSTIYSTGKVCN | |
| | PDNPQECLLLEPGLNEIMANSLDYNERLWAWESWRSEVG | |
| | KQLRPLYEEYVVLKNEMARANHYEDYGDYWRGDYEVNGV | |
| | DGYDYSRGQLIEDVEHTFEEIKPLYEHLHAYVRAKLMNA | |
| | YPSYISPIGCLPAHLLGDMWGRFWTNLYSLTVPFGQKPN | |
| | IDVTDAMVDQAWDAQRIFKEAEKFFVSVGLPNMTQGFWE | |
| | NSMLTDPGNVQKAVCHPTAWDLGKGDERILMCTKVTMDD | |
| | FLTAHHEMGHIQYDMAYAAQPELLRNGANEGFHEAVGEI | |
| | MSLSAATPKHLKSIGLLSPDFQEDNETEINFLLKQALTI | |
| | VGTLPFTYMLEKWRWMVFKGEIPKDQWMKKWWEMKREIV | |
| | GVVEPVPHDETYCDPASLFHVSNDYSFIRYYTRTLYQFQ | |
| | FQEALCQAAKHEGPLHKCDISNSTEAGQKLENMLRLGKS | |
| | EPWTLALENVVGAKNMNVRPLLNYFEPLFTWLKDONKNS | |
| | FVGWSTDWSPYADQSIKVRISLKSALGDKAYEWNDNEMY | |
| | LFRSSVAYAMRQYFLKVKNQMILFGEEDVRVANLKPRIS | |
| | FNFFVTAPKNVSDIIPRTEVEKAIRMSRSRINDAFRLND | |
| | NSLEFLGIQPTLGPPNQPPVSIWLIVFGVVMGVIVVGIV | |
| | ILIFTGIRDRKKKNKARSGENPYASIDISKGENNPGFQN | |
| | TDDVQTSF | |

It is standard in the art to number the amino acid positions within a given SARS-CoV-2 spike protein (or polypeptide) according to the corresponding amino acid position in the Wuhan-Hu-1 Spike reference sequence (SEQ ID NO: 1). As such, unless otherwise indicated, the designation of amino acid positions in the amino acid sequence of a SARS-Cov-2 spike protein (or polypeptide) (e.g., a SARS-CoV-2 spike protein immunogen (or immunogenic fragment and/or immunogenic variant thereof)) herein is relative to the amino acid sequence of SEQ ID NO: 1.

A person of ordinary skill in the art can determine the corresponding amino acid for a given amino acid position by (i) aligning the amino acid sequence of the Wuhan-Hu-1 Spike reference sequence (SEQ ID NO: 1) to the amino acid sequence of the query spike protein, (ii) locating the relevant amino acid position in the Wuhan-Hu-1 Spike reference sequence, and (iii) determining the amino acid located at the corresponding amino acid position in the query spike protein. As query spike proteins may have for example, insertions or deletions of amino acids relative to the Wuhan-Hu-1 Spike reference sequence, the amino acid corresponding to a particular position in the Wuhan-Hu-1 Spike reference sequence may not have the same amino acid number if one were to simply count the number of amino acids within the query spike protein.

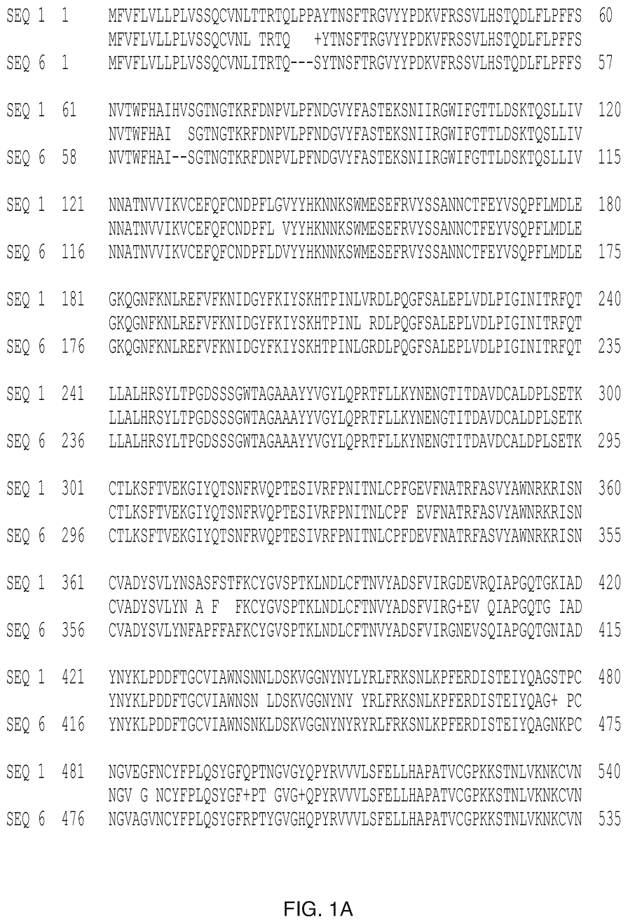

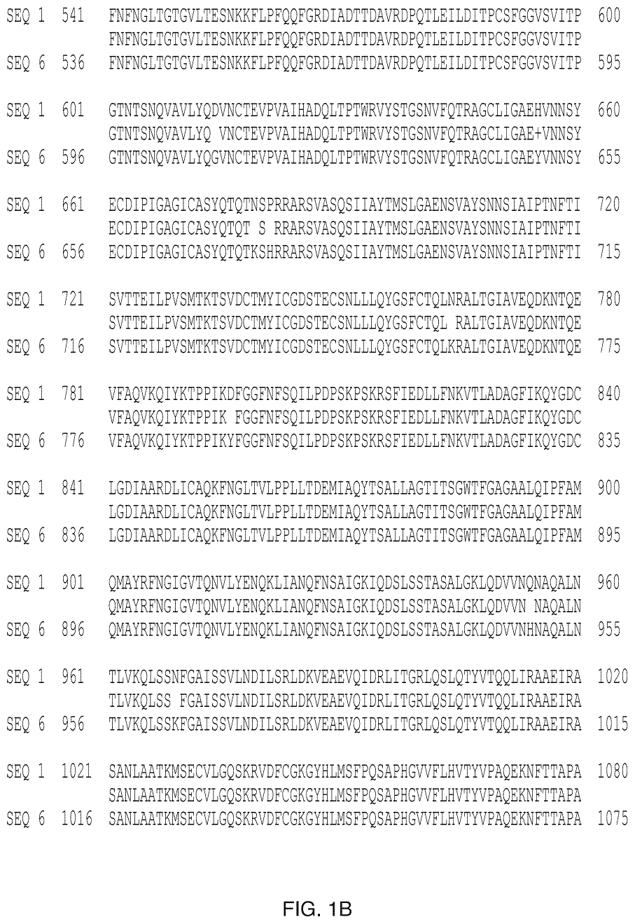

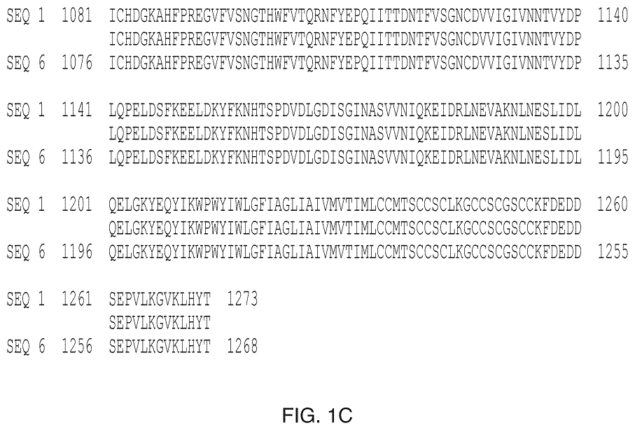

For example, FIGS. 1A-1C shows an alignment of the Wuhan-Hu-1 Spike reference sequence (SEQ ID NO: 1) to the amino acid sequence of a query spike protein (SEQ ID NO: 6). The query spike protein comprises, inter alia, a deletion of five amino acids relative to the Wuhan-Hu-1 Spike reference sequence (amino acids L24, P25, P26, H69, and V70). However, the alignment keeps the corresponding amino acids in the query spike protein (SEQ ID NO: 6) correctly aligned to the Wuhan-Hu-1 Spike reference sequence. For example, if one wanted to locate amino acid position termed 339 in a query spike protein (e.g., SEQ ID NO: 6), one would generate the alignment in FIGS. 1A-1C, locate amino acid position 339 in the Wuhan-Hu-1 Spike reference sequence (SEQ ID NO: 1) and subsequently locate the corresponding amino acid in the query spike protein (SEQ ID NO: 6), in this example position 339 (according to Wuhan-Hu-1 Spike reference sequence (SEQ ID NO: 1) numbering) in the query spike protein (SEQ ID NO: 6) is aspartic acid (D). This numbering system is indicated throughout the present disclosure by noting, “amino acid numbering relative to SEQ ID NO: 1” (or the like) and as “the Wuhan-Hu-1 Numbering System”.

Although amino acid positions of the SARS-CoV-2 spike protein (or polypeptide) are defined herein relative to the full-length immature SARS-Cov-2 spike protein that contains the native signal sequence; it should be understood that this does not require the SARS-Cov-2 spike proteins (or polypeptides) (e.g., the SARS-CoV-2 spike protein immunogens (or immunogenic fragments and/or immunogenic variants thereof)) described herein to include a native signal sequence.

As outlined above, provided herein are, inter alia, SARS-CoV-2 spike proteins (and polypeptides) (e.g., SARS-CoV-2 spike protein and polypeptide immunogens (and immunogenic fragments and/or immunogenic variants thereof)) and nucleic acid molecules encoding the same, that comprise at least one amino acid substitution (e.g., one set of amino acid substitutions described herein, see, e.g., Table 2 and Table 4). Further provided herein are SARS-CoV-2 spike proteins (and polypeptides) (e.g., SARS-CoV-2 spike protein and polypeptide immunogens (and immunogenic fragments and/or immunogenic variants thereof)) and nucleic acid molecules encoding the same, that comprise any combination of amino acid substitutions described herein (see, e.g., Table 2 and Table 4), e.g., at least one set of amino acid substitutions described in Table 2 and at least one set of amino acid substitutions described in Table 4; at least two sets of amino acid substitutions described in Table 2; at least two sets of amino acid substitutions described in Table 4, etc.

While, exemplary amino acid substitutions are provided herein, for example in Table 2 and Table 4, that specify the parental amino acid and the substituted amino acid; it should be understood that the disclosure covers the substitution of any parental amino acid with the substituted amino acid. As such, the substitutions set forth herein (e.g., in Table 2 and Table 4) include the substitution of any parental amino acid with the substituted amino acid. For example, amino acid substitution Set A set forth in Table 2 includes the substitution of any amino acid with proline at amino acid position 486 (amino acid numbering is relative to SEQ ID NO: 1).

While exemplary amino acid substitutions are provided herein, for example in Table 2 and Table 4, that specify the parental amino acid and the substituted amino acid; it is to be understood that the disclosure includes the substitution of any parental amino acid with the substituted amino acid or a physiochemically similar amino acid to the substituted amino acid set forth in Table 2 and Table 4. A person of ordinary skill in the art can determine which amino acids would be considered physiochemically similar to any given substituted amino acid set forth in Table 2 and Table 4 utilizing standard methods (e.g., based on the physiochemical properties (e.g., charge, polarity, etc.) of each amino acid).

Some sets of amino acid substitutions included in one or more of the SARS-CoV-2 spike proteins (or polypeptides) (e.g., immunogens) described herein are provided in Table 2. The amino acid numbering set forth in Table 2 is relative to SEQ ID NO: 1 (i.e., the Wuhan-Hu-1 Numbering System).

TABLE 2
Sets of Amino Acid Substitutions of SARS-
CoV-2 Spike Proteins (e.g., Immunogens)

| | | Exemplary Amino Acid Substitutions |
|---|---|---|
| Set | Amino Acid Substitutions | Relative to SEQ ID NO: 1 |
| A | 386E_485D_494P | K386E_G485D_S494P |
| B | 376D_486P_494P | T376D_F486P_S494P |
| C | 386E_452D_514M | K386E_L452D_S514M |
| D | 486P | F486P |
| E | 494P | S494P |
| F | 376D | T376D |

In some embodiments, the SARS-CoV-2 spike protein or polypeptide is an immunogen (a SARS-CoV-2 spike protein or polypeptide immunogen). In some embodiments, the SARS-CoV-2 spike protein or polypeptide comprises an immunogen (a SARS-CoV-2 spike protein or polypeptide immunogen). In some embodiments, the SARS-CoV-2 spike protein or polypeptide comprises of an immunogenic fragment of a SARS-CoV-2 spike protein. In some embodiments, the SARS-CoV-2 spike protein or polypeptide comprises an immunogenic variant of a SARS-CoV-2 spike protein. In some embodiments, the SARS-CoV-2 spike protein or polypeptide immunogen comprises an immunogenic fragment of a SARS-CoV-2 spike protein. In some embodiments, the SARS-CoV-2 spike protein or polypeptide immunogen comprises an immunogenic variant of a SARS-CoV-2 spike protein. In some embodiments, the SARS-CoV-2 spike protein or polypeptide immunogen comprises an immunogenic fragment of a SARS-CoV-2 spike protein immunogen. In some embodiments, the SARS-CoV-2 spike protein or polypeptide immunogen comprises an immunogenic variant of a SARS-CoV-2 spike protein immunogen. In some embodiments, the SARS-CoV-2 spike protein or polypeptide consists of an immunogenic fragment of a SARS-CoV-2 spike protein. In some embodiments, the SARS-CoV-2 spike protein or polypeptide consists of an immunogenic variant of a SARS-CoV-2 spike protein. In some embodiments, the SARS-CoV-2 spike protein or polypeptide immunogen comprises or consists of an immunogenic fragment of a SARS-CoV-2 spike protein. In some embodiments, the SARS-CoV-2 spike protein or polypeptide immunogen consists of an immunogenic variant of a SARS-CoV-2 spike protein. In some embodiments, the SARS-CoV-2 spike protein or polypeptide immunogen consists of an immunogenic fragment of a SARS-CoV-2 spike protein immunogen. In some embodiments, the SARS-CoV-2 spike protein or polypeptide immunogen consists of an immunogenic variant of a SARS-CoV-2 spike protein immunogen.

As defined herein, an immunogen refers to a substance that is capable of inducing an immune response (e.g., an adaptive immune response) in a subject (e.g., a human subject). The immunogenicity of a protein or polypeptide described herein (e.g., a SARS-CoV-2 spike protein or polypeptide) (or a nucleic acid molecule encoding the same) can be determined through standard in silico, in vitro, ex vivo, and in vivo assays known in the art. The type of immune response measured can be determined by a person of ordinary skill in the art, e.g., in some embodiments, an antibody based immune response and/or a T cell based immune response is evaluated. For example, a sample (e.g., a blood, cell) can be taken from a subject (e.g., an animal, human) post administration of the protein or polypeptide described herein (e.g., a SARS-CoV-2 spike protein or polypeptide) (or a nucleic acid molecule encoding the same) to be evaluated. The sample can be utilized to evaluate indicators of an immune response. For example, a blood (or serum) sample can be utilized to assess the level of antibodies that bind the antigen. Such binding can be assessed through standard ELISAs. The presence of neutralizing antibodies in the sample can also be assessed using standard assays, including, e.g., plaque reduction neutralization assays and microneutralization assays. The induction of a T-cell based immune response can be measured using standard assays known in the art, including, e.g., immunospot assays to assess cytokine production utilizing peptide stimulated peripheral blood mononuclear cells (PBMCs) from the subject. See, e.g., Corominas J. et al, Safety and immunogenicity of the protein-based PHH-1V compared to BNT162b2 as a heterologous SARS-CoV-2 booster vaccine in adults vaccinated against COVID-19: a multicentre, randomized, double-blind, non-inferiority phase IIb trial, The Lancet Regional Health Europe, Vol. 28 (100613) May 2023 DOI:https://doi.org/10.1016/j.lanepe.2023.100613; Limin D et al, Growth, Antigenicity, and Immunogenicity of SARS-CoV-2 Spike Variants Revealed by a Live rVSV-SARS-CoV-2 Virus, Frontiers in Medicine, Vol 8 (2022) DOI=10.3389/fmed.2021.793437; Hana M El Sahly and others, Humoral Immunogenicity of the mRNA-1273 Vaccine in the Phase 3 Coronavirus Efficacy (COVE) Trial, The Journal of Infectious Diseases, Volume 226, Issue 10, 15 Nov. 2022, Pages 1731-1742, https://doi.org/10.1093/infdis/jiac188; the entire contents of each of which is incorporated herein by reference for all purposes.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least 1, 2, 3, 4, 5, or 6 of the sets of amino acid substitutions set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1, 2, 3, 4, 5, or 6 of the sets of amino acid substitutions set forth in Table 2.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2, relative to the amino acid sequence set forth in any one of SEQ ID NOS: 1-5. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-5.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-5.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-5.

In some embodiments, the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2, relative to the amino acid sequence set forth in any one of SEQ ID NOS: 1-4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-4.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-4.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2, relative to the amino acid sequence set forth in any one of SEQ ID NOS: 6-10. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 6-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 6-10.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 6-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2, relative to the amino acid sequence set forth in any one of SEQ ID NOS: 6-9. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 6-9.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2, relative to the amino acid sequence set forth in any one of SEQ ID NOS: 1-10. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-10.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 2, relative to the amino acid sequence set forth in any one of SEQ ID NOS: 1-4 or 6-9. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-4 or 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-4 or 6-9.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4 or 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of at least 1, 2, 3, 4, 5, or 6 of the sets of amino acid substitutions set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of 1, 2, 3, 4, 5, or 6 of the sets of amino acid substitutions set forth in Table 2.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2, relative to the amino acid sequence set forth in any one of SEQ ID NOS: 1-5. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-5.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-5.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-5.

In some embodiments, the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2, relative to the amino acid sequence set forth in any one of SEQ ID NOS: 1-4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-4.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-4.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2, relative to the amino acid sequence set forth in any one of SEQ ID NOS: 6-10. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 6-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 6-10.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 6-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2, relative to the amino acid sequence set forth in any one of SEQ ID NOS: 6-9. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 6-9.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2, relative to the amino acid sequence set forth in any one of SEQ ID NOS: 1-10. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-10.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 2, relative to the amino acid sequence set forth in any one of SEQ ID NOS: 1-4 or 6-9. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-4 or 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-4 or 6-9.

In some embodiments, other than the at least one set of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4 or 6-9.

The amino acid sequence of exemplary SARS-CoV-2 spike proteins (e.g., immunogens) comprising at least one of the sets of amino acid substitutions set forth above in Table 2 is provided in Table 3. Table 3 provides the amino acid sequence of an exemplary RBD (SEQ ID NOS: 11-16) and the full-length spike protein (immature SEQ ID NOS: 17-22 and mature SEQ ID NOS: 23-28) of six exemplary SARS-CoV-2 spike proteins (e.g., immunogens) (A-F).

TABLE 3
Amino Acid Sequence of SARS-CoV-2 Vaccine Proteins (e.g., Immunogens)

| | | | Immature Full- | | Mature Full- | SEQ |
|---|---|---|---|---|---|---|
| | RBD Amino | SEQ | Length Amino | SEQ | Length Amino | ID |
| Description | Acid Sequence | ID NO | Acid Sequence | ID NO | Acid Sequence | NO |
| A | NITNLCPFGE | 11 | MFVFLVLLPLVSSQ | 17 | QCVNLTTRTQLP | 23 |
| | VFNATRFASV | | CVNLTTRTQLPPAY | | PAYTNSFTRGVY | |
| | YAWNRKRISN | | TNSFTRGVYYPDKV | | YPDKVFRSSVLH | |
| | CVADYSVLYN | | FRSSVLHSTQDLFL | | STQDLFLPFFSN | |
| | SASESTEKCY | | PFFSNVTWFHAIHV | | VTWFHAIHVSGT | |
| | GVSPTELNDL | | SGTNGTKRFDNPVL | | NGTKRFDNPVLP | |
| | CFTNVYADSF | | PENDGVYFASTEKS | | ENDGVYFASTEK | |
| | VIRGDEVRQI | | NIIRGWIFGTTLDS | | SNIIRGWIFGTT | |
| | APGQTGKIAD | | KTQSLLIVNNATNV | | LDSKTQSLLIVN | |
| | YNYKLPDDFT | | VIKVCEFQFCNDPF | | NATNVVIKVCEF | |
| | GCVIAWNSNN | | LGVYYHKNNKSWME | | QFCNDPFLGVYY | |
| | LDSKVGGNYN | | SEFRVYSSANNCTF | | HKNNKSWMESEF | |
| | YLYRLERKSN | | EYVSQPFLMDLEGK | | RVYSSANNCTFE | |
| | LKPFERDIST | | QGNFKNLREFVEKN | | YVSQPFLMDLEG | |
| | EIYQAGSTPC | | IDGYFKIYSKHTPI | | KQGNFKNLREFV | |
| | NGVEDENCYF | | NLVRDLPQGESALE | | FKNIDGYFKIYS | |
| | PLQPYGFQPT | | PLVDLPIGINITRE | | KHTPINLVRDLP | |
| | NGVGYQPYRV | | QTLLALHRSYLTPG | | QGFSALEPLVDL | |
| | VVLSFELLHA | | DSSSGWTAGAAAYY | | PIGINITRFQTL | |
| | PATVCGPKKS | | VGYLQPRTELLKYN | | LALHRSYLTPGD | |
| | T | | ENGTITDAVDCALD | | SSSGWTAGAAAY | |
| | | | PLSETKCTLKSFTV | | YVGYLQPRTELL | |
| | | | EKGIYQTSNERVQP | | KYNENGTITDAV | |
| | | | TESIVRFPNITNLC | | DCALDPLSETKC | |
| | | | PFGEVFNATRFASV | | TLKSFTVEKGIY | |
| | | | YAWNRKRISNCVAD | | QTSNERVOPTES | |
| | | | YSVLYNSASFSTEK | | IVRFPNITNLCP | |
| | | | CYGVSPTELNDLCF | | FGEVENATRFAS | |
| | | | TNVYADSFVIRGDE | | VYAWNRKRISNC | |
| | | | VRQIAPGQTGKIAD | | VADYSVLYNSAS | |
| | | | YNYKLPDDFTGCVI | | FSTFKCYGVSPT | |
| | | | AWNSNNLDSKVGGN | | ELNDLCFTNVYA | |
| | | | YNYLYRLERKSNLK | | DSFVIRGDEVRQ | |
| | | | PFERDISTEIYQAG | | IAPGQTGKIADY | |
| | | | STPCNGVEDENCYF | | NYKLPDDFTGCV | |
| | | | PLQPYGFQPTNGVG | | IAWNSNNLDSKV | |
| | | | YQPYRVVVLSFELL | | GGNYNYLYRLER | |
| | | | HAPATVCGPKKSTN | | KSNLKPFERDIS | |
| | | | LVKNKCVNFNENGL | | TEIYQAGSTPCN | |
| | | | TGTGVLTESNKKEL | | GVEDENCYFPLQ | |
| | | | PFQQFGRDIADTTD | | PYGFQPTNGVGY | |
| | | | AVRDPQTLEILDIT | | QPYRVVVLSFEL | |
| | | | PCSFGGVSVITPGT | | LHAPATVCGPKK | |
| | | | NTSNQVAVLYQDVN | | STNLVKNKCVNF | |
| | | | CTEVPVAIHADQLT | | NFNGLTGTGVLT | |
| | | | PTWRVYSTGSNVFQ | | ESNKKELPFQQF | |
| | | | TRAGCLIGAEHVNN | | GRDIADTTDAVR | |
| | | | SYECDIPIGAGICA | | DPQTLEILDITP | |
| | | | SYQTQTNSPRRARS | | CSFGGVSVITPG | |
| | | | VASQSIIAYTMSLG | | TNTSNQVAVLYQ | |
| | | | AENSVAYSNNSIAI | | DVNCTEVPVAIH | |
| | | | PTNFTISVTTEILP | | ADQLTPTWRVYS | |
| | | | VSMTKTSVDCTMYI | | TGSNVFQTRAGC | |
| | | | CGDSTECSNLLLQY | | LIGAEHVNNSYE | |
| | | | GSFCTQLNRALTGI | | CDIPIGAGICAS | |
| | | | AVEQDKNTQEVFAQ | | YQTQTNSPRRAR | |
| | | | VKQIYKTPPIKDFG | | SVASQSIIAYTM | |
| | | | GFNFSQILPDPSKP | | SLGAENSVAYSN | |
| | | | SKRSFIEDLLENKV | | NSIAIPTNFTIS | |
| | | | TLADAGFIKQYGDC | | VTTEILPVSMTK | |
| | | | LGDIAARDLICAQK | | TSVDCTMYICGD | |
| | | | FNGLTVLPPLLTDE | | STECSNLLLQYG | |
| | | | MIAQYTSALLAGTI | | SFCTQLNRALTG | |
| | | | TSGWTFGAGAALQI | | IAVEQDKNTQEV | |
| | | | PFAMQMAYRENGIG | | FAQVKQIYKTPP | |
| | | | VTQNVLYENQKLIA | | IKDFGGFNFSQI | |
| | | | NQFNSAIGKIQDSL | | LPDPSKPSKRSF | |
| | | | SSTASALGKLQDVV | | IEDLLENKVTLA | |
| | | | NQNAQALNTLVKQL | | DAGFIKQYGDCL | |
| | | | SSNFGAISSVLNDI | | GDIAARDLICAQ | |
| | | | LSRLDPPEAEVQID | | KFNGLTVLPPLL | |
| | | | RLITGRLQSLQTYV | | TDEMIAQYTSAL | |
| | | | TQQLIRAAEIRASA | | LAGTITSGWTFG | |
| | | | NLAATKMSECVLGQ | | AGAALQIPFAMQ | |
| | | | SKRVDFCGKGYHLM | | MAYRENGIGVTQ | |
| | | | SFPQSAPHGVVFLH | | NVLYENQKLIAN | |
| | | | VTYVPAQEKNFTTA | | QFNSAIGKIQDS | |
| | | | PAICHDGKAHFPRE | | LSSTASALGKLQ | |
| | | | GVFVSNGTHWFVTQ | | DVVNQNAQALNT | |
| | | | RNFYEPQIITTDNT | | LVKQLSSNFGAI | |
| | | | FVSGNCDVVIGIVN | | SSVLNDILSRLD | |
| | | | NTVYDPLQPELDSE | | PPEAEVQIDRLI | |
| | | | KEELDKYFKNHTSP | | TGRLQSLQTYVT | |
| | | | DVDLGDISGINASV | | QQLIRAAEIRAS | |
| | | | VNIQKEIDRLNEVA | | ANLAATKMSECV | |
| | | | KNLNESLIDLQELG | | LGQSKRVDFCGK | |
| | | | KYEQYIKWPWYIWL | | GYHLMSFPQSAP | |
| | | | GFIAGLIAIVMVTI | | HGVVFLHVTYVP | |
| | | | MLCCMTSCCSCLKG | | AQEKNFTTAPAI | |
| | | | CCSCGSCCKEDEDD | | CHDGKAHFPREG | |
| | | | SEPVLKGVKLHYT | | VFVSNGTHWFVT | |
| | | | | | QRNFYEPQIITT | |
| | | | | | DNTFVSGNCDVV | |
| | | | | | IGIVNNTVYDPL | |
| | | | | | QPELDSFKEELD | |
| | | | | | KYFKNHTSPDVD | |
| | | | | | LGDISGINASVV | |
| | | | | | NIQKEIDRLNEV | |
| | | | | | AKNLNESLIDLQ | |
| | | | | | ELGKYEQYIKWP | |
| | | | | | WYIWLGFIAGLI | |
| | | | | | AIVMVTIMLCCM | |
| | | | | | TSCCSCLKGCCS | |
| | | | | | CGSCCKEDEDDS | |
| | | | | | EPVLKGVKLHYT | |
| B | NITNLCPFGE | 12 | MFVFLVLLPLVSSQ | 18 | QCVNLTTRTQLP | 24 |
| | VFNATRFASV | | CVNLTTRTQLPPAY | | PAYTNSFTRGVY | |
| | YAWNRKRISN | | TNSFTRGVYYPDKV | | YPDKVFRSSVLH | |
| | CVADYSVLYN | | FRSSVLHSTQDLFL | | STQDLFLPFFSN | |
| | SASFSDEKCY | | PFFSNVTWFHAIHV | | VTWFHAIHVSGT | |
| | GVSPTKLNDL | | SGTNGTKRFDNPVL | | NGTKRFDNPVLP | |
| | CFTNVYADSF | | PENDGVYFASTEKS | | FNDGVYFASTEK | |
| | VIRGDEVRQI | | NIIRGWIFGTTLDS | | SNIIRGWIFGTT | |
| | APGQTGKIAD | | KTQSLLIVNNATNV | | LDSKTQSLLIVN | |
| | YNYKLPDDET | | VIKVCEFQFCNDPF | | NATNVVIKVCEF | |
| | GCVIAWNSNN | | LGVYYHKNNKSWME | | QFCNDPFLGVYY | |
| | LDSKVGGNYN | | SEFRVYSSANNCTF | | HKNNKSWMESEF | |
| | YLYRLERKSN | | EYVSQPFLMDLEGK | | RVYSSANNCTFE | |
| | LKPFERDIST | | QGNFKNLREFVEKN | | YVSQPFLMDLEG | |
| | EIYQAGSTPC | | IDGYFKIYSKHTPI | | KOGNEKNLREFV | |
| | NGVEGPNCYF | | NLVRDLPQGFSALE | | FKNIDGYFKIYS | |
| | PLQPYGFQPT | | PLVDLPIGINITRE | | KHTPINLVRDLP | |
| | NGVGYQPYRV | | QTLLALHRSYLTPG | | QGFSALEPLVDL | |
| | VVLSFELLHA | | DSSSGWTAGAAAYY | | PIGINITRFQTL | |
| | PATVCGPKKS | | VGYLQPRTFLLKYN | | LALHRSYLTPGD | |
| | T | | ENGTITDAVDCALD | | SSSGWTAGAAAY | |
| | | | PLSETKCTLKSFTV | | YVGYLQPRTELL | |
| | | | EKGIYQTSNERVQP | | KYNENGTITDAV | |
| | | | TESIVRFPNITNLC | | DCALDPLSETKC | |
| | | | PFGEVFNATRFASV | | TLKSFTVEKGIY | |
| | | | YAWNRKRISNCVAD | | QTSNERVOPTES | |
| | | | YSVLYNSASFSDEK | | IVRFPNITNLCP | |
| | | | CYGVSPTKLNDLCF | | FGEVENATRFAS | |
| | | | TNVYADSFVIRGDE | | VYAWNRKRISNC | |
| | | | VRQIAPGQTGKIAD | | VADYSVLYNSAS | |
| | | | YNYKLPDDFTGCVI | | FSDFKCYGVSPT | |
| | | | AWNSNNLDSKVGGN | | KLNDLCFTNVYA | |
| | | | YNYLYRLFRKSNLK | | DSFVIRGDEVRQ | |
| | | | PFERDISTEIYQAG | | IAPGQTGKIADY | |
| | | | STPCNGVEGPNCYF | | NYKLPDDFTGCV | |
| | | | PLQPYGFQPTNGVG | | IAWNSNNLDSKV | |
| | | | YQPYRVVVLSFELL | | GGNYNYLYRLER | |
| | | | HAPATVCGPKKSTN | | KSNLKPFERDIS | |
| | | | LVKNKCVNENENGL | | TEIYQAGSTPCN | |
| | | | TGTGVLTESNKKEL | | GVEGPNCYFPLQ | |
| | | | PFQQFGRDIADTTD | | PYGFQPTNGVGY | |
| | | | AVRDPQTLEILDIT | | QPYRVVVLSFEL | |
| | | | PCSFGGVSVITPGT | | LHAPATVCGPKK | |
| | | | NTSNQVAVLYQDVN | | STNLVKNKCVNF | |
| | | | CTEVPVAIHADQLT | | NFNGLTGTGVLT | |
| | | | PTWRVYSTGSNVFQ | | ESNKKELPFQQF | |
| | | | TRAGCLIGAEHVNN | | GRDIADTTDAVR | |
| | | | SYECDIPIGAGICA | | DPQTLEILDITP | |
| | | | SYQTQTNSPRRARS | | CSFGGVSVITPG | |
| | | | VASQSIIAYTMSLG | | TNTSNQVAVLYQ | |
| | | | AENSVAYSNNSIAI | | DVNCTEVPVAIH | |
| | | | PTNFTISVTTEILP | | ADQLTPTWRVYS | |
| | | | VSMTKTSVDCTMYI | | TGSNVFQTRAGC | |
| | | | CGDSTECSNLLLQY | | LIGAEHVNNSYE | |
| | | | GSFCTQLNRALTGI | | CDIPIGAGICAS | |
| | | | AVEQDKNTQEVFAQ | | YQTQTNSPRRAR | |
| | | | VKQIYKTPPIKDEG | | SVASQSIIAYTM | |
| | | | GFNFSQILPDPSKP | | SLGAENSVAYSN | |
| | | | SKRSFIEDLLENKV | | NSIAIPTNFTIS | |
| | | | TLADAGFIKQYGDC | | VTTEILPVSMTK | |
| | | | LGDIAARDLICAQK | | TSVDCTMYICGD | |
| | | | FNGLTVLPPLLTDE | | STECSNLLLQYG | |
| | | | MIAQYTSALLAGTI | | SFCTQLNRALTG | |
| | | | TSGWTFGAGAALQI | | IAVEQDKNTQEV | |
| | | | PFAMQMAYRENGIG | | FAQVKQIYKTPP | |
| | | | VTQNVLYENQKLIA | | IKDFGGFNFSQI | |
| | | | NQFNSAIGKIQDSL | | LPDPSKPSKRSF | |
| | | | SSTASALGKLQDVV | | IEDLLENKVTLA | |
| | | | NQNAQALNTLVKQL | | DAGFIKQYGDCL | |
| | | | SSNFGAISSVLNDI | | GDIAARDLICAQ | |
| | | | LSRLDPPEAEVQID | | KFNGLTVLPPLL | |
| | | | RLITGRLQSLQTYV | | TDEMIAQYTSAL | |
| | | | TQQLIRAAEIRASA | | LAGTITSGWTFG | |
| | | | NLAATKMSECVLGQ | | AGAALQIPFAMQ | |
| | | | SKRVDFCGKGYHLM | | MAYRENGIGVTQ | |
| | | | SFPQSAPHGVVFLH | | NVLYENQKLIAN | |
| | | | VTYVPAQEKNFTTA | | QFNSAIGKIQDS | |
| | | | PAICHDGKAHFPRE | | LSSTASALGKLQ | |
| | | | GVFVSNGTHWFVTQ | | DVVNQNAQALNT | |
| | | | RNFYEPQIITTDNT | | LVKQLSSNFGAI | |
| | | | FVSGNCDVVIGIVN | | SSVLNDILSRLD | |
| | | | NTVYDPLQPELDSE | | PPEAEVQIDRLI | |
| | | | KEELDKYFKNHTSP | | TGRLQSLQTYVT | |
| | | | DVDLGDISGINASV | | QQLIRAAEIRAS | |
| | | | VNIQKEIDRLNEVA | | ANLAATKMSECV | |
| | | | KNLNESLIDLQELG | | LGQSKRVDFCGK | |
| | | | KYEQYIKWPWYIWL | | GYHLMSFPQSAP | |
| | | | GFIAGLIAIVMVTI | | HGVVFLHVTYVP | |
| | | | MLCCMTSCCSCLKG | | AQEKNFTTAPAI | |
| | | | CCSCGSCCKEDEDD | | CHDGKAHFPREG | |
| | | | SEPVLKGVKLHYT | | VFVSNGTHWFVT | |
| | | | | | QRNFYEPQIITT | |
| | | | | | DNTFVSGNCDVV | |
| | | | | | IGIVNNTVYDPL | |
| | | | | | QPELDSFKEELD | |
| | | | | | KYFKNHTSPDVD | |
| | | | | | LGDISGINASVV | |
| | | | | | NIQKEIDRLNEV | |
| | | | | | AKNLNESLIDLQ | |
| | | | | | ELGKYEQYIKWP | |
| | | | | | WYIWLGFIAGLI | |
| | | | | | AIVMVTIMLCCM | |
| | | | | | TSCCSCLKGCCS | |
| | | | | | CGSCCKEDEDDS | |
| | | | | | EPVLKGVKLHYT | |
| C | NITNLCPFGE | 13 | MFVFLVLLPLVSSQ | 19 | QCVNLTTRTQLP | 25 |
| | VFNATRFASV | | CVNLTTRTQLPPAY | | PAYTNSFTRGVY | |
| | YAWNRKRISN | | TNSFTRGVYYPDKV | | YPDKVERSSVLH | |
| | CVADYSVLYN | | FRSSVLHSTQDLFL | | STQDLFLPFFSN | |
| | SASESTEKCY | | PFFSNVTWFHAIHV | | VTWFHAIHVSGT | |
| | GVSPTELNDL | | SGTNGTKRFDNPVL | | NGTKRFDNPVLP | |
| | CFTNVYADSF | | PENDGVYFASTEKS | | ENDGVYFASTEK | |
| | VIRGDEVRQI | | NIIRGWIFGTTLDS | | SNIIRGWIFGTT | |
| | APGQTGKIAD | | KTQSLLIVNNATNV | | LDSKTQSLLIVN | |
| | YNYKLPDDET | | VIKVCEFQFCNDPF | | NATNVVIKVCEF | |
| | GCVIAWNSNN | | LGVYYHKNNKSWME | | QFCNDPFLGVYY | |
| | LDSKVGGNYN | | SEFRVYSSANNCTE | | HKNNKSWMESEF | |
| | YDYRLERKSN | | EYVSQPFLMDLEGK | | RVYSSANNCTFE | |
| | LKPFERDIST | | QGNFKNLREFVEKN | | YVSQPFLMDLEG | |
| | EIYQAGSTPC | | IDGYFKIYSKHTPI | | KQGNFKNLREFV | |
| | NGVEGENCYF | | NLVRDLPQGFSALE | | FKNIDGYFKIYS | |
| | PLQSYGFQPT | | PLVDLPIGINITRF | | KHTPINLVRDLP | |
| | NGVGYQPYRV | | QTLLALHRSYLTPG | | QGFSALEPLVDL | |
| | VVLMFELLHA | | DSSSGWTAGAAAYY | | PIGINITRFQTL | |
| | PATVCGPKKS | | VGYLQPRTELLKYN | | LALHRSYLTPGD | |
| | T | | ENGTITDAVDCALD | | SSSGWTAGAAAY | |
| | | | PLSETKCTLKSFTV | | YVGYLQPRTELL | |
| | | | EKGIYQTSNERVQP | | KYNENGTITDAV | |
| | | | TESIVRFPNITNLC | | DCALDPLSETKC | |
| | | | PFGEVFNATRFASV | | TLKSFTVEKGIY | |
| | | | YAWNRKRISNCVAD | | QTSNERVOPTES | |
| | | | YSVLYNSASFSTEK | | IVRFPNITNLCP | |
| | | | CYGVSPTELNDLCF | | FGEVENATRFAS | |
| | | | TNVYADSFVIRGDE | | VYAWNRKRISNC | |
| | | | VRQIAPGQTGKIAD | | VADYSVLYNSAS | |
| | | | YNYKLPDDFTGCVI | | FSTFKCYGVSPT | |
| | | | AWNSNNLDSKVGGN | | ELNDLCFTNVYA | |
| | | | YNYDYRLERKSNLK | | DSFVIRGDEVRQ | |
| | | | PFERDISTEIYQAG | | IAPGQTGKIADY | |
| | | | STPCNGVEGENCYF | | NYKLPDDFTGCV | |
| | | | PLQSYGFQPTNGVG | | IAWNSNNLDSKV | |
| | | | YQPYRVVVLMFELL | | GGNYNYDYRLER | |
| | | | HAPATVCGPKKSTN | | KSNLKPFERDIS | |
| | | | LVKNKCVNENENGL | | TEIYQAGSTPCN | |
| | | | TGTGVLTESNKKEL | | GVEGENCYFPLQ | |
| | | | PFQQFGRDIADTTD | | SYGFQPTNGVGY | |
| | | | AVRDPQTLEILDIT | | QPYRVVVLMFEL | |
| | | | PCSFGGVSVITPGT | | LHAPATVCGPKK | |
| | | | NTSNQVAVLYQDVN | | STNLVKNKCVNF | |
| | | | CTEVPVAIHADQLT | | NFNGLTGTGVLT | |
| | | | PTWRVYSTGSNVFQ | | ESNKKELPFQQF | |
| | | | TRAGCLIGAEHVNN | | GRDIADTTDAVR | |
| | | | SYECDIPIGAGICA | | DPQTLEILDITP | |
| | | | SYQTQTNSPRRARS | | CSFGGVSVITPG | |
| | | | VASQSIIAYTMSLG | | TNTSNQVAVLYQ | |
| | | | AENSVAYSNNSIAI | | DVNCTEVPVAIH | |
| | | | PTNFTISVTTEILP | | ADQLTPTWRVYS | |
| | | | VSMTKTSVDCTMYI | | TGSNVFQTRAGC | |
| | | | CGDSTECSNLLLQY | | LIGAEHVNNSYE | |
| | | | GSFCTQLNRALTGI | | CDIPIGAGICAS | |
| | | | AVEQDKNTQEVFAQ | | YQTQTNSPRRAR | |
| | | | VKQIYKTPPIKDEG | | SVASQSIIAYTM | |
| | | | GFNFSQILPDPSKP | | SLGAENSVAYSN | |
| | | | SKRSFIEDLLENKV | | NSIAIPTNFTIS | |
| | | | TLADAGFIKQYGDC | | VTTEILPVSMTK | |
| | | | LGDIAARDLICAQK | | TSVDCTMYICGD | |
| | | | FNGLTVLPPLLTDE | | STECSNLLLQYG | |
| | | | MIAQYTSALLAGTI | | SFCTQLNRALTG | |
| | | | TSGWTFGAGAALQI | | IAVEQDKNTQEV | |
| | | | PFAMQMAYRENGIG | | FAQVKQIYKTPP | |
| | | | VTQNVLYENQKLIA | | IKDFGGFNFSQI | |
| | | | NQFNSAIGKIQDSL | | LPDPSKPSKRSF | |
| | | | SSTASALGKLQDVV | | IEDLLENKVTLA | |
| | | | NQNAQALNTLVKQL | | DAGFIKQYGDCL | |
| | | | SSNEGAISSVLNDI | | GDIAARDLICAQ | |
| | | | LSRLDPPEAEVQID | | KFNGLTVLPPLL | |
| | | | RLITGRLQSLQTYV | | TDEMIAQYTSAL | |
| | | | TQQLIRAAEIRASA | | LAGTITSGWTFG | |
| | | | NLAATKMSECVLGQ | | AGAALQIPFAMQ | |
| | | | SKRVDFCGKGYHLM | | MAYRENGIGVTQ | |
| | | | SFPQSAPHGVVELH | | NVLYENQKLIAN | |
| | | | VTYVPAQEKNFTTA | | QFNSAIGKIQDS | |
| | | | PAICHDGKAHFPRE | | LSSTASALGKLQ | |
| | | | GVFVSNGTHWFVTQ | | DVVNQNAQALNT | |
| | | | RNFYEPQIITTDNT | | LVKQLSSNFGAI | |
| | | | FVSGNCDVVIGIVN | | SSVLNDILSRLD | |
| | | | NTVYDPLQPELDSF | | PPEAEVQIDRLI | |
| | | | KEELDKYFKNHTSP | | TGRLQSLQTYVT | |
| | | | DVDLGDISGINASV | | QQLIRAAEIRAS | |
| | | | VNIQKEIDRLNEVA | | ANLAATKMSECV | |
| | | | KNLNESLIDLQELG | | LGQSKRVDFCGK | |
| | | | KYEQYIKWPWYIWL | | GYHLMSFPQSAP | |
| | | | GFIAGLIAIVMVTI | | HGVVFLHVTYVP | |
| | | | MLCCMTSCCSCLKG | | AQEKNFTTAPAI | |
| | | | CCSCGSCCKEDEDD | | CHDGKAHFPREG | |
| | | | SEPVLKGVKLHYT | | VFVSNGTHWFVT | |
| | | | | | QRNFYEPQIITT | |
| | | | | | DNTFVSGNCDVV | |
| | | | | | IGIVNNTVYDPL | |
| | | | | | QPELDSFKEELD | |
| | | | | | KYFKNHTSPDVD | |
| | | | | | LGDISGINASVV | |
| | | | | | NIQKEIDRLNEV | |
| | | | | | AKNLNESLIDLQ | |
| | | | | | ELGKYEQYIKWP | |
| | | | | | WYIWLGFIAGLI | |
| | | | | | AIVMVTIMLCCM | |
| | | | | | TSCCSCLKGCCS | |
| | | | | | CGSCCKFDEDDS | |
| | | | | | EPVLKGVKLHYT | |
| D | NITNLCPFGE | 14 | MFVFLVLLPLVSSQ | 20 | QCVNLTTRTQLP | 26 |
| | VFNATRFASV | | CVNLTTRTQLPPAY | | PAYTNSFTRGVY | |
| | YAWNRKRISN | | TNSFTRGVYYPDKV | | YPDKVFRSSVLH | |
| | CVADYSVLYN | | FRSSVLHSTQDLFL | | STQDLFLPFFSN | |
| | SASESTEKCY | | PFFSNVTWFHAIHV | | VTWFHAIHVSGT | |
| | GVSPTKLNDL | | SGTNGTKRFDNPVL | | NGTKRFDNPVLP | |
| | CFTNVYADSF | | PENDGVYFASTEKS | | FNDGVYFASTEK | |
| | VIRGDEVRQI | | NIIRGWIFGTTLDS | | SNIIRGWIFGTT | |
| | APGQTGKIAD | | KTQSLLIVNNATNV | | LDSKTQSLLIVN | |
| | YNYKLPDDET | | VIKVCEFQFCNDPF | | NATNVVIKVCEF | |
| | GCVIAWNSNN | | LGVYYHKNNKSWME | | QFCNDPFLGVYY | |
| | LDSKVGGNYN | | SEFRVYSSANNCTF | | HKNNKSWMESEF | |
| | YLYRLERKSN | | EYVSQPFLMDLEGK | | RVYSSANNCTFE | |
| | LKPFERDIST | | QGNFKNLREFVEKN | | YVSQPFLMDLEG | |
| | EIYQAGSTPC | | IDGYFKIYSKHTPI | | KQGNFKNLREFV | |
| | NGVEGPNCYF | | NLVRDLPQGESALE | | FKNIDGYFKIYS | |
| | PLQSYGFQPT | | PLVDLPIGINITRE | | KHTPINLVRDLP | |
| | NGVGYQPYRV | | QTLLALHRSYLTPG | | QGFSALEPLVDL | |
| | VVLSFELLHA | | DSSSGWTAGAAAYY | | PIGINITRFQTL | |
| | PATVCGPKKS | | VGYLQPRTELLKYN | | LALHRSYLTPGD | |
| | T | | ENGTITDAVDCALD | | SSSGWTAGAAAY | |
| | | | PLSETKCTLKSFTV | | YVGYLQPRTELL | |
| | | | EKGIYQTSNERVQP | | KYNENGTITDAV | |
| | | | TESIVREPNITNLC | | DCALDPLSETKC | |
| | | | PFGEVFNATRFASV | | TLKSFTVEKGIY | |
| | | | YAWNRKRISNCVAD | | QTSNERVQPTES | |
| | | | YSVLYNSASFSTEK | | IVRFPNITNLCP | |
| | | | CYGVSPTKLNDLCF | | FGEVENATRFAS | |
| | | | TNVYADSFVIRGDE | | VYAWNRKRISNC | |
| | | | VRQIAPGQTGKIAD | | VADYSVLYNSAS | |
| | | | YNYKLPDDETGCVI | | FSTFKCYGVSPT | |
| | | | AWNSNNLDSKVGGN | | KLNDLCFTNVYA | |
| | | | YNYLYRLERKSNLK | | DSFVIRGDEVRQ | |
| | | | PFERDISTEIYQAG | | IAPGQTGKIADY | |
| | | | STPCNGVEGPNCYF | | NYKLPDDFTGCV | |
| | | | PLQSYGFQPTNGVG | | IAWNSNNLDSKV | |
| | | | YQPYRVVVLSFELL | | GGNYNYLYRLER | |
| | | | HAPATVCGPKKSTN | | KSNLKPFERDIS | |
| | | | LVKNKCVNENENGL | | TEIYQAGSTPCN | |
| | | | TGTGVLTESNKKEL | | GVEGPNCYFPLQ | |
| | | | PFQQFGRDIADTTD | | SYGFQPTNGVGY | |
| | | | AVRDPQTLEILDIT | | QPYRVVVLSFEL | |
| | | | PCSFGGVSVITPGT | | LHAPATVCGPKK | |
| | | | NTSNQVAVLYQDVN | | STNLVKNKCVNE | |
| | | | CTEVPVAIHADQLT | | NFNGLTGTGVLT | |
| | | | PTWRVYSTGSNVFQ | | ESNKKFLPFQQF | |
| | | | TRAGCLIGAEHVNN | | GRDIADTTDAVR | |
| | | | SYECDIPIGAGICA | | DPQTLEILDITP | |
| | | | SYQTQTNSPRRARS | | CSFGGVSVITPG | |
| | | | VASQSIIAYTMSLG | | TNTSNQVAVLYQ | |
| | | | AENSVAYSNNSIAI | | DVNCTEVPVAIH | |
| | | | PTNFTISVTTEILP | | ADQLTPTWRVYS | |
| | | | VSMTKTSVDCTMYI | | TGSNVFQTRAGC | |
| | | | CGDSTECSNLLLQY | | LIGAEHVNNSYE | |
| | | | GSFCTQLNRALTGI | | CDIPIGAGICAS | |
| | | | AVEQDKNTQEVFAQ | | YQTQTNSPRRAR | |
| | | | VKQIYKTPPIKDEG | | SVASQSIIAYTM | |
| | | | GFNFSQILPDPSKP | | SLGAENSVAYSN | |
| | | | SKRSFIEDLLENKV | | NSIAIPTNFTIS | |
| | | | TLADAGFIKQYGDC | | VTTEILPVSMTK | |
| | | | LGDIAARDLICAQK | | TSVDCTMYICGD | |
| | | | FNGLTVLPPLLTDE | | STECSNLLLQYG | |
| | | | MIAQYTSALLAGTI | | SFCTQLNRALTG | |
| | | | TSGWTFGAGAALQI | | IAVEQDKNTQEV | |
| | | | PFAMQMAYRENGIG | | FAQVKQIYKTPP | |
| | | | VTQNVLYENQKLIA | | IKDFGGENESQI | |
| | | | NQFNSAIGKIQDSL | | LPDPSKPSKRSF | |
| | | | SSTASALGKLQDVV | | IEDLLENKVTLA | |
| | | | NQNAQALNTLVKQL | | DAGFIKQYGDCL | |
| | | | SSNFGAISSVLNDI | | GDIAARDLICAQ | |
| | | | LSRLDPPEAEVQID | | KFNGLTVLPPLL | |
| | | | RLITGRLQSLQTYV | | TDEMIAQYTSAL | |
| | | | TQQLIRAAEIRASA | | LAGTITSGWTFG | |
| | | | NLAATKMSECVLGQ | | AGAALQIPFAMQ | |
| | | | SKRVDFCGKGYHLM | | MAYRENGIGVTQ | |
| | | | SFPQSAPHGVVFLH | | NVLYENQKLIAN | |
| | | | VTYVPAQEKNFTTA | | QFNSAIGKIQDS | |
| | | | PAICHDGKAHFPRE | | LSSTASALGKLQ | |
| | | | GVFVSNGTHWFVTQ | | DVVNQNAQALNT | |
| | | | RNFYEPQIITTDNT | | LVKQLSSNFGAI | |
| | | | FVSGNCDVVIGIVN | | SSVLNDILSRLD | |
| | | | NTVYDPLQPELDSF | | PPEAEVQIDRLI | |
| | | | KEELDKYFKNHTSP | | TGRLQSLQTYVT | |
| | | | DVDLGDISGINASV | | QQLIRAAEIRAS | |
| | | | VNIQKEIDRLNEVA | | ANLAATKMSECV | |
| | | | KNLNESLIDLQELG | | LGQSKRVDFCGK | |
| | | | KYEQYIKWPWYIWL | | GYHLMSFPQSAP | |
| | | | GFIAGLIAIVMVTI | | HGVVFLHVTYVP | |
| | | | MLCCMTSCCSCLKG | | AQEKNFTTAPAI | |
| | | | CCSCGSCCKEDEDD | | CHDGKAHFPREG | |
| | | | SEPVLKGVKLHYT | | VFVSNGTHWFVT | |
| | | | | | QRNFYEPQIITT | |
| | | | | | DNTFVSGNCDVV | |
| | | | | | IGIVNNTVYDPL | |
| | | | | | QPELDSFKEELD | |
| | | | | | KYFKNHTSPDVD | |
| | | | | | LGDISGINASVV | |
| | | | | | NIQKEIDRLNEV | |
| | | | | | AKNLNESLIDLQ | |
| | | | | | ELGKYEQYIKWP | |
| | | | | | WYIWLGFIAGLI | |
| | | | | | AIVMVTIMLCCM | |
| | | | | | TSCCSCLKGCCS | |
| | | | | | CGSCCKFDEDDS | |
| | | | | | EPVLKGVKLHYT | |
| E | NITNLCPFGE | 15 | MFVFLVLLPLVSSQ | 21 | QCVNLTTRTQLP | 27 |
| | VFNATRFASV | | CVNLTTRTQLPPAY | | PAYTNSFTRGVY | |
| | YAWNRKRISN | | TNSFTRGVYYPDKV | | YPDKVFRSSVLH | |
| | CVADYSVLYN | | FRSSVLHSTQDLFL | | STQDLFLPFFSN | |
| | SASFSTFKCY | | PFFSNVTWFHAIHV | | VTWFHAIHVSGT | |
| | GVSPTKLNDL | | SGTNGTKRFDNPVL | | NGTKRFDNPVLP | |
| | CFTNVYADSF | | PENDGVYFASTEKS | | FNDGVYFASTEK | |
| | VIRGDEVRQI | | NIIRGWIFGTTLDS | | SNIIRGWIFGTT | |
| | APGQTGKIAD | | KTQSLLIVNNATNV | | LDSKTQSLLIVN | |
| | YNYKLPDDET | | VIKVCEFQFCNDPF | | NATNVVIKVCEF | |
| | GCVIAWNSNN | | LGVYYHKNNKSWME | | QFCNDPFLGVYY | |
| | LDSKVGGNYN | | SEFRVYSSANNCTF | | HKNNKSWMESEF | |
| | YLYRLERKSN | | EYVSQPFLMDLEGK | | RVYSSANNCTFE | |
| | LKPFERDIST | | QGNFKNLREFVEKN | | YVSQPFLMDLEG | |
| | EIYQAGSTPC | | IDGYFKIYSKHTPI | | KQGNEKNLREFV | |
| | NGVEGENCYF | | NLVRDLPQGESALE | | FKNIDGYFKIYS | |
| | PLQPYGFQPT | | PLVDLPIGINITRF | | KHTPINLVRDLP | |
| | NGVGYQPYRV | | QTLLALHRSYLTPG | | QGFSALEPLVDL | |
| | VVLSFELLHA | | DSSSGWTAGAAAYY | | PIGINITRFQTL | |
| | PATVCGPKKS | | VGYLQPRTELLKYN | | LALHRSYLTPGD | |
| | T | | ENGTITDAVDCALD | | SSSGWTAGAAAY | |
| | | | PLSETKCTLKSFTV | | YVGYLQPRTELL | |
| | | | EKGIYQTSNERVQP | | KYNENGTITDAV | |
| | | | TESIVRFPNITNLC | | DCALDPLSETKC | |
| | | | PFGEVFNATRFASV | | TLKSFTVEKGIY | |
| | | | YAWNRKRISNCVAD | | QTSNERVQPTES | |
| | | | YSVLYNSASFSTEK | | IVRFPNITNLCP | |
| | | | CYGVSPTKLNDLCF | | FGEVENATRFAS | |
| | | | TNVYADSFVIRGDE | | VYAWNRKRISNC | |
| | | | VRQIAPGQTGKIAD | | VADYSVLYNSAS | |
| | | | YNYKLPDDFTGCVI | | FSTFKCYGVSPT | |
| | | | AWNSNNLDSKVGGN | | KLNDLCFTNVYA | |
| | | | YNYLYRLERKSNLK | | DSFVIRGDEVRQ | |
| | | | PFERDISTEIYQAG | | IAPGQTGKIADY | |
| | | | STPCNGVEGENCYF | | NYKLPDDFTGCV | |
| | | | PLQPYGFQPTNGVG | | IAWNSNNLDSKV | |
| | | | YQPYRVVVLSFELL | | GGNYNYLYRLER | |
| | | | HAPATVCGPKKSTN | | KSNLKPFERDIS | |
| | | | LVKNKCVNFNENGL | | TEIYQAGSTPCN | |
| | | | TGTGVLTESNKKEL | | GVEGENCYFPLQ | |
| | | | PFQQFGRDIADTTD | | PYGFQPTNGVGY | |
| | | | AVRDPQTLEILDIT | | QPYRVVVLSFEL | |
| | | | PCSFGGVSVITPGT | | LHAPATVCGPKK | |
| | | | NTSNQVAVLYQDVN | | STNLVKNKCVNF | |
| | | | CTEVPVAIHADQLT | | NFNGLTGTGVLT | |
| | | | PTWRVYSTGSNVFQ | | ESNKKELPFQQF | |
| | | | TRAGCLIGAEHVNN | | GRDIADTTDAVR | |
| | | | SYECDIPIGAGICA | | DPQTLEILDITP | |
| | | | SYQTQTNSPRRARS | | CSFGGVSVITPG | |
| | | | VASQSIIAYTMSLG | | TNTSNQVAVLYQ | |
| | | | AENSVAYSNNSIAI | | DVNCTEVPVAIH | |
| | | | PTNFTISVTTEILP | | ADQLTPTWRVYS | |
| | | | VSMTKTSVDCTMYI | | TGSNVFQTRAGC | |
| | | | CGDSTECSNLLLQY | | LIGAEHVNNSYE | |
| | | | GSFCTQLNRALTGI | | CDIPIGAGICAS | |
| | | | AVEQDKNTQEVFAQ | | YQTQTNSPRRAR | |
| | | | VKQIYKTPPIKDFG | | SVASQSIIAYTM | |
| | | | GENFSQILPDPSKP | | SLGAENSVAYSN | |
| | | | SKRSFIEDLLFNKV | | NSIAIPTNFTIS | |
| | | | TLADAGFIKQYGDC | | VTTEILPVSMTK | |
| | | | LGDIAARDLICAQK | | TSVDCTMYICGD | |
| | | | FNGLTVLPPLLTDE | | STECSNLLLQYG | |
| | | | MIAQYTSALLAGTI | | SFCTQLNRALTG | |
| | | | TSGWTFGAGAALQI | | IAVEQDKNTQEV | |
| | | | PFAMQMAYRENGIG | | FAQVKQIYKTPP | |
| | | | VTQNVLYENQKLIA | | IKDFGGENESQI | |
| | | | NQFNSAIGKIQDSL | | LPDPSKPSKRSF | |
| | | | SSTASALGKLQDVV | | IEDLLENKVTLA | |
| | | | NQNAQALNTLVKQL | | DAGFIKQYGDCL | |
| | | | SSNEGAISSVLNDI | | GDIAARDLICAQ | |
| | | | LSRLDPPEAEVQID | | KENGLTVLPPLL | |
| | | | RLITGRLQSLQTYV | | TDEMIAQYTSAL | |
| | | | TQQLIRAAEIRASA | | LAGTITSGWTFG | |
| | | | NLAATKMSECVLGQ | | AGAALQIPFAMQ | |
| | | | SKRVDFCGKGYHLM | | MAYRENGIGVTQ | |
| | | | SFPQSAPHGVVELH | | NVLYENQKLIAN | |
| | | | VTYVPAQEKNFTTA | | QFNSAIGKIQDS | |
| | | | PAICHDGKAHFPRE | | LSSTASALGKLQ | |
| | | | GVFVSNGTHWFVTQ | | DVVNQNAQALNT | |
| | | | RNFYEPQIITTDNT | | LVKQLSSNFGAI | |
| | | | FVSGNCDVVIGIVN | | SSVLNDILSRLD | |
| | | | NTVYDPLQPELDSE | | PPEAEVQIDRLI | |
| | | | KEELDKYFKNHTSP | | TGRLQSLQTYVT | |
| | | | DVDLGDISGINASV | | QQLIRAAEIRAS | |
| | | | VNIQKEIDRLNEVA | | ANLAATKMSECV | |
| | | | KNLNESLIDLQELG | | LGQSKRVDFCGK | |
| | | | KYEQYIKWPWYIWL | | GYHLMSFPQSAP | |
| | | | GFIAGLIAIVMVTI | | HGVVFLHVTYVP | |
| | | | MLCCMTSCCSCLKG | | AQEKNFTTAPAI | |
| | | | CCSCGSCCKFDEDD | | CHDGKAHFPREG | |
| | | | SEPVLKGVKLHYT | | VFVSNGTHWFVT | |
| | | | | | QRNFYEPQIITT | |
| | | | | | DNTFVSGNCDVV | |
| | | | | | IGIVNNTVYDPL | |
| | | | | | QPELDSFKEELD | |
| | | | | | KYFKNHTSPDVD | |
| | | | | | LGDISGINASVV | |
| | | | | | NIQKEIDRLNEV | |
| | | | | | AKNLNESLIDLQ | |
| | | | | | ELGKYEQYIKWP | |
| | | | | | WYIWLGFIAGLI | |
| | | | | | AIVMVTIMLCCM | |
| | | | | | TSCCSCLKGCCS | |
| | | | | | CGSCCKEDEDDS | |
| | | | | | EPVLKGVKLHYT | |
| F | NITNLCPFGE | 16 | MFVFLVLLPLVSSQ | 22 | QCVNLTTRTQLP | 28 |
| | VFNATRFASV | | CVNLTTRTQLPPAY | | PAYTNSFTRGVY | |
| | YAWNRKRISN | | TNSFTRGVYYPDKV | | YPDKVERSSVLH | |
| | CVADYSVLYN | | FRSSVLHSTQDLFL | | STQDLFLPFFSN | |
| | SASFSDEKCY | | PFFSNVTWFHAIHV | | VTWFHAIHVSGT | |
| | GVSPTKLNDL | | SGTNGTKRFDNPVL | | NGTKRFDNPVLP | |
| | CFTNVYADSF | | PENDGVYFASTEKS | | FNDGVYFASTEK | |
| | VIRGDEVRQI | | NIIRGWIFGTTLDS | | SNIIRGWIFGTT | |
| | APGQTGKIAD | | KTQSLLIVNNATNV | | LDSKTQSLLIVN | |
| | YNYKLPDDET | | VIKVCEFQFCNDPF | | NATNVVIKVCEF | |
| | GCVIAWNSNN | | LGVYYHKNNKSWME | | QFCNDPFLGVYY | |
| | LDSKVGGNYN | | SEFRVYSSANNCTF | | HKNNKSWMESEF | |
| | YLYRLFRKSN | | EYVSQPFLMDLEGK | | RVYSSANNCTFE | |
| | LKPFERDIST | | QGNFKNLREFVEKN | | YVSQPFLMDLEG | |
| | EIYQAGSTPC | | IDGYFKIYSKHTPI | | KQGNFKNLREFV | |
| | NGVEGENCYF | | NLVRDLPQGFSALE | | FKNIDGYFKIYS | |
| | PLQSYGFQPT | | PLVDLPIGINITRE | | KHTPINLVRDLP | |
| | NGVGYQPYRV | | QTLLALHRSYLTPG | | QGFSALEPLVDL | |
| | VVLSFELLHA | | DSSSGWTAGAAAYY | | PIGINITRFQTL | |
| | PATVCGPKKS | | VGYLQPRTFLLKYN | | LALHRSYLTPGD | |
| | T | | ENGTITDAVDCALD | | SSSGWTAGAAAY | |
| | | | PLSETKCTLKSFTV | | YVGYLQPRTELL | |
| | | | EKGIYQTSNFRVQP | | KYNENGTITDAV | |
| | | | TESIVRFPNITNLC | | DCALDPLSETKC | |
| | | | PFGEVFNATRFASV | | TLKSFTVEKGIY | |
| | | | YAWNRKRISNCVAD | | QTSNERVQPTES | |
| | | | YSVLYNSASESDEK | | IVRFPNITNLCP | |
| | | | CYGVSPTKLNDLCF | | FGEVENATRFAS | |
| | | | TNVYADSFVIRGDE | | VYAWNRKRISNC | |
| | | | VRQIAPGQTGKIAD | | VADYSVLYNSAS | |
| | | | YNYKLPDDFTGCVI | | FSDFKCYGVSPT | |
| | | | AWNSNNLDSKVGGN | | KLNDLCFTNVYA | |
| | | | YNYLYRLERKSNLK | | DSFVIRGDEVRQ | |
| | | | PFERDISTEIYQAG | | IAPGQTGKIADY | |
| | | | STPCNGVEGENCYF | | NYKLPDDFTGCV | |
| | | | PLQSYGFQPTNGVG | | IAWNSNNLDSKV | |
| | | | YQPYRVVVLSFELL | | GGNYNYLYRLER | |
| | | | HAPATVCGPKKSTN | | KSNLKPFERDIS | |
| | | | LVKNKCVNENENGL | | TEIYQAGSTPCN | |
| | | | TGTGVLTESNKKEL | | GVEGENCYFPLQ | |
| | | | PFQQFGRDIADTTD | | SYGFQPTNGVGY | |
| | | | AVRDPQTLEILDIT | | QPYRVVVLSFEL | |
| | | | PCSFGGVSVITPGT | | LHAPATVCGPKK | |
| | | | NTSNQVAVLYQDVN | | STNLVKNKCVNF | |
| | | | CTEVPVAIHADQLT | | NFNGLTGTGVLT | |
| | | | PTWRVYSTGSNVFQ | | ESNKKELPFQQF | |
| | | | TRAGCLIGAEHVNN | | GRDIADTTDAVR | |
| | | | SYECDIPIGAGICA | | DPQTLEILDITP | |
| | | | SYQTQTNSPRRARS | | CSFGGVSVITPG | |
| | | | VASQSIIAYTMSLG | | TNTSNQVAVLYQ | |
| | | | AENSVAYSNNSIAI | | DVNCTEVPVAIH | |
| | | | PTNFTISVTTEILP | | ADQLTPTWRVYS | |
| | | | VSMTKTSVDCTMYI | | TGSNVFQTRAGC | |
| | | | CGDSTECSNLLLQY | | LIGAEHVNNSYE | |
| | | | GSFCTQLNRALTGI | | CDIPIGAGICAS | |
| | | | AVEQDKNTQEVFAQ | | YQTQTNSPRRAR | |
| | | | VKQIYKTPPIKDFG | | SVASQSIIAYTM | |
| | | | GENFSQILPDPSKP | | SLGAENSVAYSN | |
| | | | SKRSFIEDLLENKV | | NSIAIPTNFTIS | |
| | | | TLADAGFIKQYGDC | | VTTEILPVSMTK | |
| | | | LGDIAARDLICAQK | | TSVDCTMYICGD | |
| | | | FNGLTVLPPLLTDE | | STECSNLLLQYG | |
| | | | MIAQYTSALLAGTI | | SFCTQLNRALTG | |
| | | | TSGWTFGAGAALQI | | IAVEQDKNTQEV | |
| | | | PFAMQMAYRENGIG | | FAQVKQIYKTPP | |
| | | | VTQNVLYENQKLIA | | IKDFGGFNFSQI | |
| | | | NQFNSAIGKIQDSL | | LPDPSKPSKRSF | |
| | | | SSTASALGKLQDVV | | IEDLLENKVTLA | |
| | | | NQNAQALNTLVKQL | | DAGFIKQYGDCL | |
| | | | SSNEGAISSVLNDI | | GDIAARDLICAQ | |
| | | | LSRLDPPEAEVQID | | KFNGLTVLPPLL | |
| | | | RLITGRLQSLQTYV | | TDEMIAQYTSAL | |
| | | | TQQLIRAAEIRASA | | LAGTITSGWTFG | |
| | | | NLAATKMSECVLGQ | | AGAALQIPFAMQ | |
| | | | SKRVDFCGKGYHLM | | MAYRENGIGVTQ | |
| | | | SFPQSAPHGVVELH | | NVLYENQKLIAN | |
| | | | VTYVPAQEKNFTTA | | QFNSAIGKIQDS | |
| | | | PAICHDGKAHFPRE | | LSSTASALGKLQ | |
| | | | GVFVSNGTHWFVTQ | | DVVNQNAQALNT | |
| | | | RNFYEPQIITTDNT | | LVKQLSSNFGAI | |
| | | | FVSGNCDVVIGIVN | | SSVLNDILSRLD | |
| | | | NTVYDPLQPELDSF | | PPEAEVQIDRLI | |
| | | | KEELDKYFKNHTSP | | TGRLQSLQTYVT | |
| | | | DVDLGDISGINASV | | QQLIRAAEIRAS | |
| | | | VNIQKEIDRLNEVA | | ANLAATKMSECV | |
| | | | KNLNESLIDLQELG | | LGQSKRVDFCGK | |
| | | | KYEQYIKWPWYIWL | | GYHLMSFPQSAP | |
| | | | GFIAGLIAIVMVTI | | HGVVFLHVTYVP | |
| | | | MLCCMTSCCSCLKG | | AQEKNFTTAPAI | |
| | | | CCSCGSCCKEDEDD | | CHDGKAHFPREG | |
| | | | SEPVLKGVKLHYT | | VFVSNGTHWFVT | |
| | | | | | QRNFYEPQIITT | |
| | | | | | DNTFVSGNCDVV | |
| | | | | | IGIVNNTVYDPL | |
| | | | | | QPELDSFKEELD | |
| | | | | | KYFKNHTSPDVD | |
| | | | | | LGDISGINASVV | |
| | | | | | NIQKEIDRLNEV | |
| | | | | | AKNLNESLIDLQ | |
| | | | | | ELGKYEQYIKWP | |
| | | | | | WYIWLGFIAGLI | |
| | | | | | AIVMVTIMLCCM | |
| | | | | | TSCCSCLKGCCS | |
| | | | | | CGSCCKFDEDDS | |
| | | | | | EPVLKGVKLHYT | |

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of any SARS-CoV-2 protein set forth in Table 3. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises or consists of at least 1, 2, 3, 4, 5, or 6 of the sets of amino acid substitutions set forth in Table 2, and the other than the at least 1, 2, 3, 4, 5, or 6 of the sets of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any SARS-Cov-2 protein set forth in Table 3.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 11-28. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises or consists of at least 1, 2, 3, 4, 5, or 6 of the sets of amino acid substitutions set forth in Table 2, and the other than the at least 1, 2, 3, 4, 5, or 6 of the sets of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 11-28.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 11-16. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises or consists of at least 1, 2, 3, 4, 5, or 6 of the sets of amino acid substitutions set forth in Table 2, and the other than the at least 1, 2, 3, 4, 5, or 6 of the sets of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 11-16.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 17-28. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises or consists of at least 1, 2, 3, 4, 5, or 6 of the sets of amino acid substitutions set forth in Table 2, and the other than the at least 1, 2, 3, 4, 5, or 6 of the sets of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 17-28.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 17-22. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises or consists of at least 1, 2, 3, 4, 5, or 6 of the sets of amino acid substitutions set forth in Table 2, and the other than the at least 1, 2, 3, 4, 5, or 6 of the sets of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 17-22.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 23-28. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises or consists of at least 1, 2, 3, 4, 5, or 6 of the sets of amino acid substitutions set forth in Table 2, and the other than the at least 1, 2, 3, 4, 5, or 6 of the sets of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 23-28.

Additional sets of amino acid substitutions included in one or more of the SARS-CoV-2 spike proteins (e.g., immunogens) described herein are provided in Table 4. The amino acid numbering set forth in Table 4 are relative to a reference SARS-CoV-2 spike protein comprising the amino acid sequence of SEQ ID NO: 1 (i.e., the Wuhan-Hu-1 Numbering System).

TABLE 4
Amino Acid Substitutions in SARS-CoV-2
Spike Proteins (e.g., Immunogens)

| | | Exemplary Amino Acid |
|---|---|---|
| | | Substitutions |
| | | Relative to the amino acid |
| Set | Amino Acid Substitutions | sequence of SEQ ID NO: 6 |
| G | 339H_346T_356T_417T_{—} | D339H_R346T_K356T_N417T_{—} |
| | 444T_460K | K444T_N460K |
| H | 339H_446S | D339H_G446S |
| I | 417Y_444E | N417Y_K444E |
| J | 417H_444N | N417H_K444N |
| K | 417I_444M | N417I_K444M |
| L | 444Q | K444Q |
| M | 346S_445D | R346S_V445D |
| N | 445G_450T | V445G_N450T |
| O | 339H_346T_356T_417T_{—} | D339H_R346T_K356T_N417T_{—} |
| | 444T_460K_396T_468N | K444T_N460K_Y396T_I468N |
| P | 339H_446S_396T_468N | D339H_G446S_Y396T_I468N |
| Q | 417Y_444E_396T_468N | N417Y_K444E_Y396T_I468N |
| R | 417H_444N_396T_468N | N417H_K444N_Y396T_I468N |
| S | 417I_444M_396T_468N | N417I_K444M_Y396T_I468N |
| T | 444Q_396T_468N | K444Q_Y396T_I468N |
| U | 346S_445D_396T_468N | R346S_V445D_Y396T_I468N |
| V | 445G_450T_396T_468N | V445G_N450T_Y396T_I468N |

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least 1, 2, 3, 4, 5, 6, 7, 8, 9, 10, 11, 12, 13, 14, 15, or 16 sets of amino acid substitutions set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1, 2, 3, 4, 5, 6, 7, 8, 9, 10, 11, 12, 13, 14, 15, or 16 sets of amino acid substitutions set forth in Table 4.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereofl)) comprises one or more variations that are not set forth in Table 4, relative to the amino acid sequence set forth in any one of SEQ ID NOS: 6-10. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 6-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 6-10.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 6-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 4, relative to the amino acid sequence set forth in any one of SEQ ID NOS: 6-9. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 6-9.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 4, relative to the amino acid sequence set forth in any one of SEQ ID NOS: 6-10. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 1-5.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-5.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-5.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 4. In some embodiments, the SARS-CoV-2 spike protein (e.g., the SARS-CoV-2 spike protein immunogen (or the immunogenic fragment and/or immunogenic variant thereof)) comprises one or more variations that are not set forth in Table 4, relative to the amino acid sequence set forth in any one of SEQ ID NOS: 6-9. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 1-5.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-4.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 4, relative to the amino acid sequence set forth in any one of SEQ ID NOS: 1-4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 1-4.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-10.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises one or more variations that are not set forth in Table 4, relative to the amino acid sequence set forth in any one of SEQ ID NOS: 1-4 or 6-9. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 1-4 or 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-4 or 6-9.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4 or 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of at least 1, 2, 3, 4, 5, 6, 7, 8, 9, 10, 11, 12, 13, 14, 15, or 16 sets of amino acid substitutions set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of 1, 2, 3, 4, 5, 6, 7, 8, 9, 10, 11, 12, 13, 14, 15, or 16 sets of amino acid substitutions set forth in Table 4.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 4, relative to the amino acid sequence set forth in any one of SEQ ID NOS: 6-10. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 6-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 6-10.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 6-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 4, relative to the amino acid sequence set forth in any one of SEQ ID NOS: 6-9. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 6-9.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of comprises one or more variations that are not set forth in Table 4, relative to the amino acid sequence set forth in any one of SEQ ID NOS: 6-10. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 1-5.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-5.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-5.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 4. In some embodiments, the SARS-CoV-2 spike protein (e.g., the SARS-CoV-2 spike protein immunogen (or the immunogenic fragment and/or immunogenic variant thereof)) comprises one or more variations that are not set forth in Table 4, relative to the amino acid sequence set forth in any one of SEQ ID NOS: 6-9. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 1-5.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-4.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 4, relative to the amino acid sequence set forth in any one of SEQ ID NOS: 1-4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 1-4.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-10.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-10.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of one or more variations that are not set forth in Table 4, relative to the amino acid sequence set forth in any one of SEQ ID NOS: 1-4 or 6-9. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 1-4 or 6-9.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-4 or 6-9.

In some embodiments, other than the at least one set of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4 or 6-9.

The amino acid sequence of exemplary SARS-CoV-2 spike proteins (e.g., immunogens) comprising at least one of the sets of amino acid substitutions set forth above in Table 4 is provided in Table 5. Table 5 provides the amino acid sequence of exemplary receptor binding domain (SEQ ID NOS: 29-44) and the full-length spike protein (immature SEQ ID NOS: 45-60 and mature SEQ ID NOS: 61-76) of sixteen exemplary SARS-CoV-2 proteins (e.g., immunogens) (G-V).

TABLE 5
Amino Acid Sequence of SARS-CoV-2 Vaccine Proteins (e.g., Immunogens)

| | RBD Amino | SEQ | Immature Full- | SEQ | Mature Full- | SEQ |
|---|---|---|---|---|---|---|
| | Acid | ID | Length Amino | ID | Length Amino | ID |
| Description | Sequence | NO | Acid Sequence | NO | Acid Sequence | NO |
| G | NITNLCPFH | 29 | MFVFLVLLPLVSSQ | 45 | QCVNLITRTQSY | 61 |
| | EVENATTFA | | CVNLITRTQSYTNS | | TNSFTRGVYYPD | |
| | SVYAWNRTR | | FTRGVYYPDKVERS | | KVERSSVLHSTQ | |
| | ISNCVADYS | | SVLHSTQDLFLPFF | | DLFLPFFSNVTW | |
| | VLYNFAPFF | | SNVTWFHAISGTNG | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVYADSFVI | | WIFGTTLDSKTQSL | | GWIFGTTLDSKT | |
| | RGNEVSQIA | | LIVNNATNVVIKVC | | QSLLIVNNATNV | |
| | PGQTGTIAD | | EFQFCNDPFLDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSTVGG | | PFLMDLEGKQGNEK | | ANNCTFEYVSQP | |
| | NYNYRYRLE | | NLREFVEKNIDGYF | | FLMDLEGKQGNF | |
| | RKSKLKPFE | | KIYSKHTPINLGRD | | KNLREFVEKNID | |
| | RDISTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFHEV | | PLSETKCTLKSF | |
| | | | FNATTFASVYAWNR | | TVEKGIYQTSNF | |
| | | | TRISNCVADYSVLY | | RVQPTESIVREP | |
| | | | NFAPFFAFKCYGVS | | NITNLCPFHEVE | |
| | | | PTKLNDLCFTNVYA | | NATTFASVYAWN | |
| | | | DSFVIRGNEVSQIA | | RTRISNCVADYS | |
| | | | PGQTGTIADYNYKL | | VLYNFAPFFAFK | |
| | | | PDDFTGCVIAWNSN | | CYGVSPTKLNDL | |
| | | | KLDSTVGGNYNYRY | | CFTNVYADSFVI | |
| | | | RLERKSKLKPFERD | | RGNEVSQIAPGQ | |
| | | | ISTEIYQAGNKPCN | | TGTIADYNYKLP | |
| | | | GVAGVNCYFPLQSY | | DDFTGCVIAWNS | |
| | | | GFRPTYGVGHQPYR | | NKLDSTVGGNYN | |
| | | | VVVLSFELLHAPAT | | YRYRLFRKSKLK | |
| | | | VCGPKKSTNLVKNK | | PFERDISTEIYQ | |
| | | | CVNFNFNGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSFG | | VVLSFELLHAPA | |
| | | | GVSVITPGTNTSNQ | | TVCGPKKSTNLV | |
| | | | VAVLYQGVNCTEVP | | KNKCVNFNENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSEGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGFNFS | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRF | |
| | | | FCGKGYHLMSFPQS | | NGIGVTQNVLYE | |
| | | | APHGVVFLHVTYVP | | NQKLIANQENSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVF | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| H | NITNLCPFH | 30 | MFVFLVLLPLVSSQ | 46 | QCVNLITRTQSY | 62 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGTNG | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVYADSFVI | | WIFGTTLDSKTQSL | | GWIFGTTLDSKT | |
| | RGNEVSQIA | | LIVNNATNVVIKVC | | QSLLIVNNATNV | |
| | PGQTGNIAD | | EFQFCNDPFLDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSKVSG | | PFLMDLEGKQGNEK | | ANNCTFEYVSQP | |
| | NYNYRYRLE | | NLREFVFKNIDGYF | | FLMDLEGKQGNF | |
| | RKSNLKPFE | | KIYSKHTPINLGRD | | KNLREFVEKNID | |
| | RDISTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTELLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTFLLKYNEN | |
| | KST | | QTSNERVQPTESIV | | GTITDAVDCALD | |
| | | | RFPNITNLCPFHEV | | PLSETKCTLKSF | |
| | | | FNATRFASVYAWNR | | TVEKGIYQTSNF | |
| | | | KRISNCVADYSVLY | | RVQPTESIVREP | |
| | | | NFAPFFAFKCYGVS | | NITNLCPFHEVE | |
| | | | PTKLNDLCFTNVYA | | NATRFASVYAWN | |
| | | | DSFVIRGNEVSQIA | | RKRISNCVADYS | |
| | | | PGQTGNIADYNYKL | | VLYNFAPFFAFK | |
| | | | PDDETGCVIAWNSN | | CYGVSPTKLNDL | |
| | | | KLDSKVSGNYNYRY | | CFTNVYADSFVI | |
| | | | RLFRKSNLKPFERD | | RGNEVSQIAPGQ | |
| | | | ISTEIYQAGNKPCN | | TGNIADYNYKLP | |
| | | | GVAGVNCYFPLQSY | | DDFTGCVIAWNS | |
| | | | GFRPTYGVGHQPYR | | NKLDSKVSGNYN | |
| | | | VVVLSFELLHAPAT | | YRYRLERKSNLK | |
| | | | VCGPKKSTNLVKNK | | PFERDISTEIYQ | |
| | | | CVNFNFNGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSFG | | VVLSFELLHAPA | |
| | | | GVSVITPGTNTSNQ | | TVCGPKKSTNLV | |
| | | | VAVLYQGVNCTEVP | | KNKCVNENENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSFGG | |
| | | | TKSHRRARSVASQS | | VSVITPGTNTSN | |
| | | | IIAYTMSLGAENSV | | QVAVLYQGVNCT | |
| | | | AYSNNSIAIPTNET | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRE | |
| | | | FCGKGYHLMSFPQS | | NGIGVTQNVLYE | |
| | | | APHGVVFLHVTYVP | | NQKLIANQFNSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVE | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTEV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| I | NITNLCPFD | 31 | MFVFLVLLPLVSSQ | 47 | QCVNLITRTQSY | 63 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGTNG | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVYADSFVI | | WIFGTTLDSKTQSL | | GWIFGTTLDSKT | |
| | RGNEVSQIA | | LIVNNATNVVIKVC | | QSLLIVNNATNV | |
| | PGQTGYIAD | | EFQFCNDPELDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSEVGG | | PFLMDLEGKQGNEK | | ANNCTFEYVSQP | |
| | NYNYRYRLE | | NLREFVEKNIDGYF | | FLMDLEGKQGNF | |
| | RKSNLKPFE | | KIYSKHTPINLGRD | | KNLREFVEKNID | |
| | RDISTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFDEV | | PLSETKCTLKSF | |
| | | | FNATRFASVYAWNR | | TVEKGIYQTSNF | |
| | | | KRISNCVADYSVLY | | RVQPTESIVREP | |
| | | | NFAPFFAFKCYGVS | | NITNLCPFDEVE | |
| | | | PTKLNDLCFTNVYA | | NATRFASVYAWN | |
| | | | DSFVIRGNEVSQIA | | RKRISNCVADYS | |
| | | | PGQTGYIADYNYKL | | VLYNFAPFFAFK | |
| | | | PDDETGCVIAWNSN | | CYGVSPTKLNDL | |
| | | | KLDSEVGGNYNYRY | | CFTNVYADSEVI | |
| | | | RLERKSNLKPFERD | | RGNEVSQIAPGQ | |
| | | | ISTEIYQAGNKPCN | | TGYIADYNYKLP | |
| | | | GVAGVNCYFPLQSY | | DDFTGCVIAWNS | |
| | | | GFRPTYGVGHQPYR | | NKLDSEVGGNYN | |
| | | | VVVLSFELLHAPAT | | YRYRLFRKSNLK | |
| | | | VCGPKKSTNLVKNK | | PFERDISTEIYQ | |
| | | | CVNFNENGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSFG | | VVLSFELLHAPA | |
| | | | GVSVITPGTNTSNQ | | TVCGPKKSTNLV | |
| | | | VAVLYQGVNCTEVP | | KNKCVNFNENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSFGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRE | |
| | | | FCGKGYHLMSFPQS | | NGIGVTQNVLYE | |
| | | | APHGVVFLHVTYVP | | NQKLIANQFNSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVE | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| J | NITNLCPED | 32 | MFVFLVLLPLVSSQ | 48 | QCVNLITRTQSY | 64 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGING | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVYADSFVI | | WIFGTTLDSKTQSL | | GWIFGTTLDSKT | |
| | RGNEVSQIA | | LIVNNATNVVIKVC | | QSLLIVNNATNV | |
| | PGQTGHIAD | | EFQFCNDPELDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSNVGG | | PFLMDLEGKQGNEK | | ANNCTFEYVSQP | |
| | NYNYRYRLE | | NLREFVEKNIDGYF | | FLMDLEGKQGNF | |
| | RKSNLKPFE | | KIYSKHTPINLGRD | | KNLREFVEKNID | |
| | RDISTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFDEV | | PLSETKCTLKSF | |
| | | | FNATRFASVYAWNR | | TVEKGIYQTSNF | |
| | | | KRISNCVADYSVLY | | RVQPTESIVREP | |
| | | | NFAPFFAFKCYGVS | | NITNLCPFDEVE | |
| | | | PTKLNDLCFTNVYA | | NATRFASVYAWN | |
| | | | DSFVIRGNEVSQIA | | RKRISNCVADYS | |
| | | | PGQTGHIADYNYKL | | VLYNFAPFFAFK | |
| | | | PDDFTGCVIAWNSN | | CYGVSPTKLNDL | |
| | | | KLDSNVGGNYNYRY | | CFTNVYADSEVI | |
| | | | RLFRKSNLKPFERD | | RGNEVSQIAPGQ | |
| | | | ISTEIYQAGNKPCN | | TGHIADYNYKLP | |
| | | | GVAGVNCYFPLQSY | | DDFTGCVIAWNS | |
| | | | GFRPTYGVGHQPYR | | NKLDSNVGGNYN | |
| | | | VVVLSFELLHAPAT | | YRYRLFRKSNLK | |
| | | | VCGPKKSTNLVKNK | | PFERDISTEIYQ | |
| | | | CVNFNFNGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSEG | | VVLSFELLHAPA | |
| | | | GVSVITPGTNTSNQ | | TVCGPKKSTNLV | |
| | | | VAVLYQGVNCTEVP | | KNKCVNENENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSFGG | |
| | | | TKSHRRARSVASQS | | VSVITPGTNTSN | |
| | | | IIAYTMSLGAENSV | | QVAVLYQGVNCT | |
| | | | AYSNNSIAIPTNET | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRF | |
| | | | FCGKGYHLMSFPQS | | NGIGVTQNVLYE | |
| | | | APHGVVFLHVTYVP | | NQKLIANQENSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVE | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| K | NITNLCPED | 33 | MFVFLVLLPLVSSQ | 49 | QCVNLITRTQSY | 65 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGTNG | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVYADSFVI | | WIFGTTLDSKTQSL | | GWIFGTTLDSKT | |
| | RGNEVSQIA | | LIVNNATNVVIKVC | | QSLLIVNNATNV | |
| | PGQTGIIAD | | EFQFCNDPFLDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSMVGG | | PFLMDLEGKQGNEK | | ANNCTFEYVSQP | |
| | NYNYRYRLE | | NLREFVEKNIDGYF | | FLMDLEGKQGNF | |
| | RKSNLKPFE | | KIYSKHTPINLGRD | | KNLREFVEKNID | |
| | RDISTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFDEV | | PLSETKCTLKSF | |
| | | | FNATRFASVYAWNR | | TVEKGIYQTSNF | |
| | | | KRISNCVADYSVLY | | RVQPTESIVREP | |
| | | | NFAPFFAFKCYGVS | | NITNLCPFDEVE | |
| | | | PTKLNDLCFTNVYA | | NATRFASVYAWN | |
| | | | DSFVIRGNEVSQIA | | RKRISNCVADYS | |
| | | | PGQTGIIADYNYKL | | VLYNFAPFFAFK | |
| | | | PDDFTGCVIAWNSN | | CYGVSPTKLNDL | |
| | | | KLDSMVGGNYNYRY | | CFTNVYADSFVI | |
| | | | RLFRKSNLKPFERD | | RGNEVSQIAPGQ | |
| | | | ISTEIYQAGNKPCN | | TGIIADYNYKLP | |
| | | | GVAGVNCYFPLQSY | | DDFTGCVIAWNS | |
| | | | GFRPTYGVGHQPYR | | NKLDSMVGGNYN | |
| | | | VVVLSFELLHAPAT | | YRYRLFRKSNLK | |
| | | | VCGPKKSTNLVKNK | | PFERDISTEIYQ | |
| | | | CVNFNENGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSFG | | VVLSFELLHAPA | |
| | | | GVSVITPGTNTSNQ | | TVCGPKKSTNLV | |
| | | | VAVLYQGVNCTEVP | | KNKCVNENENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSFGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRE | |
| | | | FCGKGYHLMSFPQS | | NGIGVTQNVLYE | |
| | | | APHGVVFLHVTYVP | | NQKLIANQENSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVF | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| L | NITNLCPED | 34 | MFVFLVLLPLVSSQ | 50 | QCVNLITRTQSY | 66 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGTNG | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVYADSEVI | | WIFGTTLDSKTQSL | | GWIFGTTLDSKT | |
| | RGNEVSQIA | | LIVNNATNVVIKVC | | QSLLIVNNATNV | |
| | PGQTGNIAD | | EFQFCNDPELDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSQVGG | | PFLMDLEGKQGNEK | | ANNCTFEYVSQP | |
| | NYNYRYRLE | | NLREFVEKNIDGYF | | FLMDLEGKQGNF | |
| | RKSNLKPFE | | KIYSKHTPINLGRD | | KNLREFVFKNID | |
| | RDISTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFDEV | | PLSETKCTLKSF | |
| | | | FNATRFASVYAWNR | | TVEKGIYQTSNF | |
| | | | KRISNCVADYSVLY | | RVQPTESIVREP | |
| | | | NFAPFFAFKCYGVS | | NITNLCPFDEVE | |
| | | | PTKLNDLCFTNVYA | | NATRFASVYAWN | |
| | | | DSFVIRGNEVSQIA | | RKRISNCVADYS | |
| | | | PGQTGNIADYNYKL | | VLYNFAPFFAFK | |
| | | | PDDETGCVIAWNSN | | CYGVSPTKLNDL | |
| | | | KLDSQVGGNYNYRY | | CFTNVYADSFVI | |
| | | | RLFRKSNLKPFERD | | RGNEVSQIAPGQ | |
| | | | ISTEIYQAGNKPCN | | TGNIADYNYKLP | |
| | | | GVAGVNCYFPLQSY | | DDETGCVIAWNS | |
| | | | GFRPTYGVGHQPYR | | NKLDSQVGGNYN | |
| | | | VVVLSFELLHAPAT | | YRYRLFRKSNLK | |
| | | | VCGPKKSTNLVKNK | | PFERDISTEIYQ | |
| | | | CVNFNENGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSFG | | VVLSFELLHAPA | |
| | | | GVSVITPGTNTSNQ | | TVCGPKKSTNLV | |
| | | | VAVLYQGVNCTEVP | | KNKCVNENENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSFGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQTY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | ENKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRE | |
| | | | FCGKGYHLMSFPQS | | NGIGVTQNVLYE | |
| | | | APHGVVFLHVTYVP | | NQKLIANQENSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVE | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| M | NITNLCPED | 35 | MFVFLVLLPLVSSQ | 51 | QCVNLITRTQSY | 67 |
| | EVENATSFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGING | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVYADSFVI | | WIFGTTLDSKTQSL | | GWIFGTTLDSKT | |
| | RGNEVSQIA | | LIVNNATNVVIKVC | | QSLLIVNNATNV | |
| | PGQTGNIAD | | EFQFCNDPELDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSKDGG | | PFLMDLEGKQGNEK | | ANNCTFEYVSQP | |
| | NYNYRYRLE | | NLREFVEKNIDGYF | | FLMDLEGKQGNF | |
| | RKSNLKPFE | | KIYSKHTPINLGRD | | KNLREFVEKNID | |
| | RDISTEIYQ | | LPQGFSALEPLVDL | | GYFKIYSKHTPI | |
| | AGNKPCNGV | | PIGINITRFQTLLA | | NLGRDLPQGFSA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFDEV | | PLSETKCTLKSF | |
| | | | FNATSFASVYAWNR | | TVEKGIYQTSNF | |
| | | | KRISNCVADYSVLY | | RVQPTESIVREP | |
| | | | NEAPFFAFKCYGVS | | NITNLCPFDEVE | |
| | | | PTKLNDLCFTNVYA | | NATSFASVYAWN | |
| | | | DSFVIRGNEVSQIA | | RKRISNCVADYS | |
| | | | PGQTGNIADYNYKL | | VLYNFAPFFAFK | |
| | | | PDDFTGCVIAWNSN | | CYGVSPTKLNDL | |
| | | | KLDSKDGGNYNYRY | | CFTNVYADSFVI | |
| | | | RLFRKSNLKPFERD | | RGNEVSQIAPGQ | |
| | | | ISTEIYQAGNKPCN | | TGNIADYNYKLP | |
| | | | GVAGVNCYFPLQSY | | DDFTGCVIAWNS | |
| | | | GFRPTYGVGHQPYR | | NKLDSKDGGNYN | |
| | | | VVVLSFELLHAPAT | | YRYRLFRKSNLK | |
| | | | VCGPKKSTNLVKNK | | PFERDISTEIYQ | |
| | | | CVNFNFNGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSEG | | VVLSFELLHAPA | |
| | | | GVSVITPGTNTSNQ | | TVCGPKKSTNLV | |
| | | | VAVLYQGVNCTEVP | | KNKCVNFNENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSEGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRE | |
| | | | FCGKGYHLMSFPQS | | NGIGVTQNVLYE | |
| | | | APHGVVFLHVTYVP | | NQKLIANQFNSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVF | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| N | NITNLCPED | 36 | MFVFLVLLPLVSSQ | 52 | QCVNLITRTQSY | 68 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFE | | SNVTWFHAISGING | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVYADSFVI | | WIFGTTLDSKTQSL | | GWIFGTTLDSKT | |
| | RGNEVSQIA | | LIVNNATNVVIKVC | | QSLLIVNNATNV | |
| | PGQTGNIAD | | EFQFCNDPELDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSKGGG | | PFLMDLEGKQGNEK | | ANNCTFEYVSQP | |
| | NYTYRYRLF | | NLREFVEKNIDGYF | | FLMDLEGKQGNF | |
| | RKSNLKPFE | | KIYSKHTPINLGRD | | KNLREFVEKNID | |
| | RDISTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFDEV | | PLSETKCTLKSF | |
| | | | FNATRFASVYAWNR | | TVEKGIYQTSNE | |
| | | | KRISNCVADYSVLY | | RVQPTESIVREP | |
| | | | NFAPFFAFKCYGVS | | NITNLCPFDEVE | |
| | | | PTKLNDLCFTNVYA | | NATRFASVYAWN | |
| | | | DSFVIRGNEVSQIA | | RKRISNCVADYS | |
| | | | PGQTGNIADYNYKL | | VLYNFAPFFAFK | |
| | | | PDDFTGCVIAWNSN | | CYGVSPTKLNDL | |
| | | | KLDSKGGGNYTYRY | | CFTNVYADSEVI | |
| | | | RLERKSNLKPFERD | | RGNEVSQIAPGQ | |
| | | | ISTEIYQAGNKPCN | | TGNIADYNYKLP | |
| | | | GVAGVNCYFPLQSY | | DDFTGCVIAWNS | |
| | | | GFRPTYGVGHQPYR | | NKLDSKGGGNYT | |
| | | | VVVLSFELLHAPAT | | YRYRLFRKSNLK | |
| | | | VCGPKKSTNLVKNK | | PFERDISTEIYQ | |
| | | | CVNFNFNGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSFG | | VVLSFELLHAPA | |
| | | | GVSVITPGTNTSNQ | | TVCGPKKSTNLV | |
| | | | VAVLYQGVNCTEVP | | KNKCVNENENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSFGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRF | |
| | | | FCGKGYHLMSFPQS | | NGIGVTQNVLYE | |
| | | | APHGVVFLHVTYVP | | NQKLIANQENSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVF | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| O | NITNLCPFH | 37 | MFVFLVLLPLVSSQ | 53 | QCVNLITRTQSY | 69 |
| | EVENATTFA | | CVNLITRTQSYTNS | | TNSFTRGVYYPD | |
| | SVYAWNRTR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGING | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVTADSFVI | | WIFGTTLDSKTQSL | | GWIFGTTLDSKT | |
| | RGNEVSQIA | | LIVNNATNVVIKVC | | QSLLIVNNATNV | |
| | PGQTGTIAD | | EFQFCNDPFLDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSTVGG | | PFLMDLEGKQGNEK | | ANNCTFEYVSQP | |
| | NYNYRYRLE | | NLREFVEKNIDGYF | | FLMDLEGKQGNF | |
| | RKSKLKPFE | | KIYSKHTPINLGRD | | KNLREFVEKNID | |
| | RDNSTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTFLLKYNEN | |
| | KST | | QTSNERVQPTESIV | | GTITDAVDCALD | |
| | | | RFPNITNLCPFHEV | | PLSETKCTLKSF | |
| | | | FNATTFASVYAWNR | | TVEKGIYQTSNE | |
| | | | TRISNCVADYSVLY | | RVQPTESIVREP | |
| | | | NFAPFFAFKCYGVS | | NITNLCPFHEVE | |
| | | | PTKLNDLCFTNVTA | | NATTFASVYAWN | |
| | | | DSFVIRGNEVSQIA | | RTRISNCVADYS | |
| | | | PGQTGTIADYNYKL | | VLYNFAPFFAFK | |
| | | | PDDETGCVIAWNSN | | CYGVSPTKLNDL | |
| | | | KLDSTVGGNYNYRY | | CFTNVTADSEVI | |
| | | | RLFRKSKLKPFERD | | RGNEVSQIAPGQ | |
| | | | NSTEIYQAGNKPCN | | TGTIADYNYKLP | |
| | | | GVAGVNCYFPLQSY | | DDFTGCVIAWNS | |
| | | | GFRPTYGVGHQPYR | | NKLDSTVGGNYN | |
| | | | VVVLSFELLHAPAT | | YRYRLFRKSKLK | |
| | | | VCGPKKSTNLVKNK | | PFERDNSTEIYQ | |
| | | | CVNFNENGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSFG | | VVLSFELLHAPA | |
| | | | GVSVITPGTNTSNQ | | TVCGPKKSTNLV | |
| | | | VAVLYQGVNCTEVP | | KNKCVNENENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSEGG | |
| | | | TKSHRRARSVASQS | | VSVITPGTNTSN | |
| | | | IIAYTMSLGAENSV | | QVAVLYQGVNCT | |
| | | | AYSNNSIAIPTNET | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GENFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRF | |
| | | | FCGKGYHLMSFPQS | | NGIGVTQNVLYE | |
| | | | APHGVVFLHVTYVP | | NQKLIANQENSA | |
| | | | AQEKNFTTAPAICH | | IGKIQDSLSSTA | |
| | | | DGKAHFPREGVFVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVE | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| P | NITNLCPFH | 38 | MFVFLVLLPLVSSQ | 54 | QCVNLITRTQSY | 70 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGTNG | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVTADSEVI | | WIFGTTLDSKTQSL | | GWIFGTTLDSKT | |
| | RGNEVSQIA | | LIVNNATNVVIKVC | | QSLLIVNNATNV | |
| | PGQTGNIAD | | EFQFCNDPELDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSKVSG | | PFLMDLEGKQGNEK | | ANNCTFEYVSQP | |
| | NYNYRYRLE | | NLREFVEKNIDGYF | | FLMDLEGKQGNE | |
| | RKSNLKPFE | | KIYSKHTPINLGRD | | KNLREFVEKNID | |
| | RDNSTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFHEV | | PLSETKCTLKSF | |
| | | | FNATRFASVYAWNR | | TVEKGIYQTSNF | |
| | | | KRISNCVADYSVLY | | RVQPTESIVREP | |
| | | | NFAPFFAFKCYGVS | | NITNLCPFHEVE | |
| | | | PTKLNDLCFTNVTA | | NATRFASVYAWN | |
| | | | DSFVIRGNEVSQIA | | RKRISNCVADYS | |
| | | | PGQTGNIADYNYKL | | VLYNFAPFFAFK | |
| | | | PDDFTGCVIAWNSN | | CYGVSPTKLNDL | |
| | | | KLDSKVSGNYNYRY | | CFTNVTADSEVI | |
| | | | RLFRKSNLKPFERD | | RGNEVSQIAPGQ | |
| | | | NSTEIYQAGNKPCN | | TGNIADYNYKLP | |
| | | | GVAGVNCYFPLQSY | | DDFTGCVIAWNS | |
| | | | GFRPTYGVGHQPYR | | NKLDSKVSGNYN | |
| | | | VVVLSFELLHAPAT | | YRYRLERKSNLK | |
| | | | VCGPKKSTNLVKNK | | PFERDNSTEIYQ | |
| | | | CVNFNENGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSEG | | VVLSFELLHAPA | |
| | | | GVSVITPGTNTSNQ | | TVCGPKKSTNLV | |
| | | | VAVLYQGVNCTEVP | | KNKCVNENENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSEGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRE | |
| | | | FCGKGYHLMSFPQS | | NGIGVTQNVLYE | |
| | | | APHGVVFLHVTYVP | | NQKLIANQFNSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVE | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| Q | NITNLCPED | 39 | MFVFLVLLPLVSSQ | 55 | QCVNLITRTQSY | 71 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGING | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVTADSEVI | | WIFGTTLDSKTQSL | | GWIFGTTLDSKT | |
| | RGNEVSQIA | | LIVNNATNVVIKVC | | QSLLIVNNATNV | |
| | PGQTGYIAD | | EFQFCNDPELDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSEVGG | | PFLMDLEGKQGNEK | | ANNCTFEYVSQP | |
| | NYNYRYRLF | | NLREFVEKNIDGYF | | FLMDLEGKQGNF | |
| | RKSNLKPFE | | KIYSKHTPINLGRD | | KNLREFVEKNID | |
| | RDNSTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFDEV | | PLSETKCTLKSF | |
| | | | FNATRFASVYAWNR | | TVEKGIYQTSNF | |
| | | | KRISNCVADYSVLY | | RVQPTESIVREP | |
| | | | NFAPFFAFKCYGVS | | NITNLCPFDEVE | |
| | | | PTKLNDLCFTNVTA | | NATRFASVYAWN | |
| | | | DSFVIRGNEVSQIA | | RKRISNCVADYS | |
| | | | PGQTGYIADYNYKL | | VLYNFAPFFAFK | |
| | | | PDDFTGCVIAWNSN | | CYGVSPTKLNDL | |
| | | | KLDSEVGGNYNYRY | | CFTNVTADSEVI | |
| | | | RLFRKSNLKPFERD | | RGNEVSQIAPGQ | |
| | | | NSTEIYQAGNKPCN | | TGYIADYNYKLP | |
| | | | GVAGVNCYFPLQSY | | DDFTGCVIAWNS | |
| | | | GFRPTYGVGHQPYR | | NKLDSEVGGNYN | |
| | | | VVVLSFELLHAPAT | | YRYRLFRKSNLK | |
| | | | VCGPKKSTNLVKNK | | PFERDNSTEIYQ | |
| | | | CVNFNFNGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSFG | | VVLSFELLHAPA | |
| | | | GVSVITPGTNTSNQ | | TVCGPKKSTNLV | |
| | | | VAVLYQGVNCTEVP | | KNKCVNENENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSFGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRF | |
| | | | FCGKGYHLMSFPQS | | NGIGVTQNVLYE | |
| | | | APHGVVFLHVTYVP | | NQKLIANQFNSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVE | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTEV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| R | NITNLCPFD | 40 | MFVFLVLLPLVSSQ | 56 | QCVNLITRTQSY | 72 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGTNG | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVTADSEVI | | WIFGTTLDSKTQSL | | GWIFGTTLDSKT | |
| | RGNEVSQIA | | LIVNNATNVVIKVC | | QSLLIVNNATNV | |
| | PGQTGHIAD | | EFQFCNDPFLDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSNVGG | | PFLMDLEGKQGNEK | | ANNCTFEYVSQP | |
| | NYNYRYRLF | | NLREFVEKNIDGYF | | FLMDLEGKQGNF | |
| | RKSNLKPFE | | KIYSKHTPINLGRD | | KNLREFVEKNID | |
| | RDNSTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFDEV | | PLSETKCTLKSF | |
| | | | FNATRFASVYAWNR | | TVEKGIYQTSNF | |
| | | | KRISNCVADYSVLY | | RVQPTESIVREP | |
| | | | NEAPFFAFKCYGVS | | NITNLCPFDEVE | |
| | | | PTKLNDLCFTNVTA | | NATRFASVYAWN | |
| | | | DSFVIRGNEVSQIA | | RKRISNCVADYS | |
| | | | PGQTGHIADYNYKL | | VLYNFAPFFAFK | |
| | | | PDDETGCVIAWNSN | | CYGVSPTKLNDL | |
| | | | KLDSNVGGNYNYRY | | CFTNVTADSFVI | |
| | | | RLFRKSNLKPFERD | | RGNEVSQIAPGQ | |
| | | | NSTEIYQAGNKPCN | | TGHIADYNYKLP | |
| | | | GVAGVNCYFPLQSY | | DDFTGCVIAWNS | |
| | | | GFRPTYGVGHQPYR | | NKLDSNVGGNYN | |
| | | | VVVLSFELLHAPAT | | YRYRLFRKSNLK | |
| | | | VCGPKKSTNLVKNK | | PFERDNSTEIYQ | |
| | | | CVNFNENGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSFG | | VVLSFELLHAPA | |
| | | | GVSVITPGTNTSNQ | | TVCGPKKSTNLV | |
| | | | VAVLYQGVNCTEVP | | KNKCVNFNENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSFGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GENFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRE | |
| | | | FCGKGYHLMSFPQS | | NGIGVTQNVLYE | |
| | | | APHGVVFLHVTYVP | | NQKLIANQENSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVF | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| S | NITNLCPED | 41 | MFVFLVLLPLVSSQ | 57 | QCVNLITRTQSY | 73 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGTNG | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVTADSEVI | | WIFGTTLDSKTQSL | | GWIFGTTLDSKT | |
| | RGNEVSQIA | | LIVNNATNVVIKVC | | QSLLIVNNATNV | |
| | PGQTGIIAD | | EFQFCNDPFLDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSMVGG | | PFLMDLEGKQGNEK | | ANNCTFEYVSQP | |
| | NYNYRYRLF | | NLREFVEKNIDGYF | | FLMDLEGKQGNF | |
| | RKSNLKPFE | | KIYSKHTPINLGRD | | KNLREFVEKNID | |
| | RDNSTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTELLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTFLLKYNEN | |
| | KST | | QTSNERVQPTESIV | | GTITDAVDCALD | |
| | | | RFPNITNLCPFDEV | | PLSETKCTLKSF | |
| | | | FNATRFASVYAWNR | | TVEKGIYQTSNF | |
| | | | KRISNCVADYSVLY | | RVQPTESIVREP | |
| | | | NFAPFFAFKCYGVS | | NITNLCPFDEVE | |
| | | | PTKLNDLCFTNVTA | | NATRFASVYAWN | |
| | | | DSFVIRGNEVSQIA | | RKRISNCVADYS | |
| | | | PGQTGIIADYNYKL | | VLYNFAPFFAFK | |
| | | | PDDFTGCVIAWNSN | | CYGVSPTKLNDL | |
| | | | KLDSMVGGNYNYRY | | CFTNVTADSFVI | |
| | | | RLERKSNLKPFERD | | RGNEVSQIAPGQ | |
| | | | NSTEIYQAGNKPCN | | TGIIADYNYKLP | |
| | | | GVAGVNCYFPLQSY | | DDFTGCVIAWNS | |
| | | | GFRPTYGVGHQPYR | | NKLDSMVGGNYN | |
| | | | VVVLSFELLHAPAT | | YRYRLFRKSNLK | |
| | | | VCGPKKSTNLVKNK | | PFERDNSTEIYQ | |
| | | | CVNFNENGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSFG | | VVLSFELLHAPA | |
| | | | GVSVITPGTNTSNQ | | TVCGPKKSTNLV | |
| | | | VAVLYQGVNCTEVP | | KNKCVNFNENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSFGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKEG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRF | |
| | | | FCGKGYHLMSFPQS | | NGIGVTQNVLYE | |
| | | | APHGVVFLHVTYVP | | NQKLIANQENSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVE | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTEV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| T | NITNLCPED | 42 | MFVFLVLLPLVSSQ | 58 | QCVNLITRTQSY | 74 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGTNG | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVTADSFVI | | WIFGTTLDSKTQSL | | GWIFGTTLDSKT | |
| | RGNEVSQIA | | LIVNNATNVVIKVC | | QSLLIVNNATNV | |
| | PGQTGNIAD | | EFQFCNDPELDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSQVGG | | PFLMDLEGKQGNEK | | ANNCTFEYVSQP | |
| | NYNYRYRLE | | NLREFVEKNIDGYF | | FLMDLEGKQGNF | |
| | RKSNLKPFE | | KIYSKHTPINLGRD | | KNLREFVEKNID | |
| | RDNSTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFDEV | | PLSETKCTLKSF | |
| | | | FNATRFASVYAWNR | | TVEKGIYQTSNF | |
| | | | KRISNCVADYSVLY | | RVQPTESIVREP | |
| | | | NFAPFFAFKCYGVS | | NITNLCPFDEVE | |
| | | | PTKLNDLCFTNVTA | | NATRFASVYAWN | |
| | | | DSFVIRGNEVSQIA | | RKRISNCVADYS | |
| | | | PGQTGNIADYNYKL | | VLYNFAPFFAFK | |
| | | | PDDFTGCVIAWNSN | | CYGVSPTKLNDL | |
| | | | KLDSQVGGNYNYRY | | CFTNVTADSEVI | |
| | | | RLFRKSNLKPFERD | | RGNEVSQIAPGQ | |
| | | | NSTEIYQAGNKPCN | | TGNIADYNYKLP | |
| | | | GVAGVNCYFPLQSY | | DDFTGCVIAWNS | |
| | | | GFRPTYGVGHQPYR | | NKLDSQVGGNYN | |
| | | | VVVLSFELLHAPAT | | YRYRLFRKSNLK | |
| | | | VCGPKKSTNLVKNK | | PFERDNSTEIYQ | |
| | | | CVNFNENGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSEG | | VVLSFELLHAPA | |
| | | | GVSVITPGTNTSNQ | | TVCGPKKSTNLV | |
| | | | VAVLYQGVNCTEVP | | KNKCVNFNENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSFGG | |
| | | | TKSHRRARSVASQS | | VSVITPGTNTSN | |
| | | | IIAYTMSLGAENSV | | QVAVLYQGVNCT | |
| | | | AYSNNSTAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRF | |
| | | | FCGKGYHLMSFPQS | | NGIGVTQNVLYE | |
| | | | APHGVVFLHVTYVP | | NQKLIANQENSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVF | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |
| U | NITNLCPED | 43 | MFVFLVLLPLVSSQ | 59 | QCVNLITRTQSY | 75 |
| | EVENATSFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGING | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVTADSEVI | | WIFGTTLDSKTQSL | | GWIFGTTLDSKT | |
| | RGNEVSQIA | | LIVNNATNVVIKVC | | QSLLIVNNATNV | |
| | PGQTGNIAD | | EFQFCNDPFLDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSKDGG | | PFLMDLEGKQGNEK | | ANNCTFEYVSQP | |
| | NYNYRYRLE | | NLREFVFKNIDGYF | | FLMDLEGKQGNF | |
| | RKSNLKPFE | | KIYSKHTPINLGRD | | KNLREFVFKNID | |
| | RDNSTEIYQ | | LPQGFSALEPLVDL | | GYFKIYSKHTPI | |
| | AGNKPCNGV | | PIGINITRFQTLLA | | NLGRDLPQGFSA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNERVQPTESIV | | GTITDAVDCALD | |
| | | | RFPNITNLCPFDEV | | PLSETKCTLKSF | |
| | | | FNATSFASVYAWNR | | TVEKGIYQTSNF | |
| | | | KRISNCVADYSVLY | | RVQPTESIVREP | |
| | | | NFAPFFAFKCYGVS | | NITNLCPFDEVE | |
| | | | PTKLNDLCFTNVTA | | NATSFASVYAWN | |
| | | | DSFVIRGNEVSQIA | | RKRISNCVADYS | |
| | | | PGQTGNIADYNYKL | | VLYNFAPFFAFK | |
| | | | PDDFTGCVIAWNSN | | CYGVSPTKLNDL | |
| | | | KLDSKDGGNYNYRY | | CFTNVTADSEVI | |
| | | | RLFRKSNLKPFERD | | RGNEVSQIAPGQ | |
| | | | NSTEIYQAGNKPCN | | TGNIADYNYKLP | |
| | | | GVAGVNCYFPLQSY | | DDFTGCVIAWNS | |
| | | | GFRPTYGVGHQPYR | | NKLDSKDGGNYN | |
| | | | VVVLSFELLHAPAT | | YRYRLERKSNLK | |
| | | | VCGPKKSTNLVKNK | | PFERDNSTEIYQ | |
| | | | CVNFNFNGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSFG | | VVLSFELLHAPA | |
| | | | GVSVITPGTNTSNQ | | TVCGPKKSTNLV | |
| | | | VAVLYQGVNCTEVP | | KNKCVNFNENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSEGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSF | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GFNFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRF | |
| | | | FCGKGYHLMSFPQS | | NGIGVTQNVLYE | |
| | | | APHGVVFLHVTYVP | | NQKLIANQFNSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVE | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KEDEDDSEPVLK | |
| | | | | | GVKLHYT | |
| V | NITNLCPED | 44 | MFVFLVLLPLVSSQ | 60 | QCVNLITRTQSY | 76 |
| | EVENATRFA | | CVNLITRTQSYTNS | | TNSFTRGVYYPD | |
| | SVYAWNRKR | | FTRGVYYPDKVERS | | KVFRSSVLHSTQ | |
| | ISNCVADYS | | SVLHSTQDLFLPFF | | DLFLPFFSNVTW | |
| | VLYNFAPFF | | SNVTWFHAISGTNG | | FHAISGTNGTKR | |
| | AFKCYGVSP | | TKRFDNPVLPENDG | | FDNPVLPENDGV | |
| | TKLNDLCFT | | VYFASTEKSNIIRG | | YFASTEKSNIIR | |
| | NVTADSEVI | | WIFGTTLDSKTQSL | | GWIFGTTLDSKT | |
| | RGNEVSQIA | | LIVNNATNVVIKVC | | QSLLIVNNATNV | |
| | PGQTGNIAD | | EFQFCNDPELDVYY | | VIKVCEFQFCND | |
| | YNYKLPDDE | | HKNNKSWMESEFRV | | PFLDVYYHKNNK | |
| | TGCVIAWNS | | YSSANNCTFEYVSQ | | SWMESEFRVYSS | |
| | NKLDSKGGG | | PFLMDLEGKQGNEK | | ANNCTFEYVSQP | |
| | NYTYRYRLF | | NLREFVEKNIDGYF | | FLMDLEGKQGNF | |
| | RKSNLKPFE | | KIYSKHTPINLGRD | | KNLREFVFKNID | |
| | RDNSTEIYQ | | LPQGFSALEPLVDL | | GYFKIYSKHTPI | |
| | AGNKPCNGV | | PIGINITRFQTLLA | | NLGRDLPQGESA | |
| | AGVNCYFPL | | LHRSYLTPGDSSSG | | LEPLVDLPIGIN | |
| | QSYGFRPTY | | WTAGAAAYYVGYLQ | | ITRFQTLLALHR | |
| | GVGHQPYRV | | PRTFLLKYNENGTI | | SYLTPGDSSSGW | |
| | VVLSFELLH | | TDAVDCALDPLSET | | TAGAAAYYVGYL | |
| | APATVCGPK | | KCTLKSFTVEKGIY | | QPRTELLKYNEN | |
| | KST | | QTSNFRVQPTESIV | | GTITDAVDCALD | |
| | | | RFPNITNLCPFDEV | | PLSETKCTLKSF | |
| | | | FNATRFASVYAWNR | | TVEKGIYQTSNF | |
| | | | KRISNCVADYSVLY | | RVQPTESIVREP | |
| | | | NFAPFFAFKCYGVS | | NITNLCPFDEVE | |
| | | | PTKLNDLCFTNVTA | | NATRFASVYAWN | |
| | | | DSFVIRGNEVSQIA | | RKRISNCVADYS | |
| | | | PGQTGNIADYNYKL | | VLYNFAPFFAFK | |
| | | | PDDFTGCVIAWNSN | | CYGVSPTKLNDL | |
| | | | KLDSKGGGNYTYRY | | CFTNVTADSFVI | |
| | | | RLFRKSNLKPFERD | | RGNEVSQIAPGQ | |
| | | | NSTEIYQAGNKPCN | | TGNIADYNYKLP | |
| | | | GVAGVNCYFPLQSY | | DDFTGCVIAWNS | |
| | | | GFRPTYGVGHQPYR | | NKLDSKGGGNYT | |
| | | | VVVLSFELLHAPAT | | YRYRLFRKSNLK | |
| | | | VCGPKKSTNLVKNK | | PFERDNSTEIYQ | |
| | | | CVNFNFNGLTGTGV | | AGNKPCNGVAGV | |
| | | | LTESNKKELPFQQF | | NCYFPLQSYGER | |
| | | | GRDIADTTDAVRDP | | PTYGVGHQPYRV | |
| | | | QTLEILDITPCSEG | | VVLSFELLHAPA | |
| | | | GVSVITPGTNTSNQ | | TVCGPKKSTNLV | |
| | | | VAVLYQGVNCTEVP | | KNKCVNENENGL | |
| | | | VAIHADQLTPTWRV | | TGTGVLTESNKK | |
| | | | YSTGSNVFQTRAGC | | FLPFQQFGRDIA | |
| | | | LIGAEYVNNSYECD | | DTTDAVRDPQTL | |
| | | | IPIGAGICASYQTQ | | EILDITPCSFGG | |
| | | | TKSHRRARSVASQS | | VSVITPGTNTSN | |
| | | | IIAYTMSLGAENSV | | QVAVLYQGVNCT | |
| | | | AYSNNSIAIPTNFT | | EVPVAIHADQLT | |
| | | | ISVTTEILPVSMTK | | PTWRVYSTGSNV | |
| | | | TSVDCTMYICGDST | | FQTRAGCLIGAE | |
| | | | ECSNLLLQYGSFCT | | YVNNSYECDIPI | |
| | | | QLKRALTGIAVEQD | | GAGICASYQTQT | |
| | | | KNTQEVFAQVKQIY | | KSHRRARSVASQ | |
| | | | KTPPIKYFGGENES | | SIIAYTMSLGAE | |
| | | | QILPDPSKPSKRSE | | NSVAYSNNSIAI | |
| | | | IEDLLENKVTLADA | | PTNFTISVTTEI | |
| | | | GFIKQYGDCLGDIA | | LPVSMTKTSVDC | |
| | | | ARDLICAQKENGLT | | TMYICGDSTECS | |
| | | | VLPPLLTDEMIAQY | | NLLLQYGSFCTQ | |
| | | | TSALLAGTITSGWT | | LKRALTGIAVEQ | |
| | | | FGAGAALQIPFAMQ | | DKNTQEVFAQVK | |
| | | | MAYRENGIGVTQNV | | QIYKTPPIKYFG | |
| | | | LYENQKLIANQENS | | GENFSQILPDPS | |
| | | | AIGKIQDSLSSTAS | | KPSKRSFIEDLL | |
| | | | ALGKLQDVVNHNAQ | | FNKVTLADAGFI | |
| | | | ALNTLVKQLSSKFG | | KQYGDCLGDIAA | |
| | | | AISSVLNDILSRLD | | RDLICAQKENGL | |
| | | | PPEAEVQIDRLITG | | TVLPPLLTDEMI | |
| | | | RLQSLQTYVTQQLI | | AQYTSALLAGTI | |
| | | | RAAEIRASANLAAT | | TSGWTFGAGAAL | |
| | | | KMSECVLGQSKRVD | | QIPFAMQMAYRF | |
| | | | FCGKGYHLMSFPQS | | NGIGVTQNVLYE | |
| | | | APHGVVFLHVTYVP | | NQKLIANQFNSA | |
| | | | AQEKNFTTAPAICH | | IGKIQDSLSSTA | |
| | | | DGKAHFPREGVEVS | | SALGKLQDVVNH | |
| | | | NGTHWFVTQRNFYE | | NAQALNTLVKQL | |
| | | | PQIITTDNTFVSGN | | SSKFGAISSVLN | |
| | | | CDVVIGIVNNTVYD | | DILSRLDPPEAE | |
| | | | PLQPELDSFKEELD | | VQIDRLITGRLQ | |
| | | | KYFKNHTSPDVDLG | | SLQTYVTQQLIR | |
| | | | DISGINASVVNIQK | | AAEIRASANLAA | |
| | | | EIDRLNEVAKNLNE | | TKMSECVLGQSK | |
| | | | SLIDLQELGKYEQY | | RVDFCGKGYHLM | |
| | | | IKWPWYIWLGFIAG | | SFPQSAPHGVVE | |
| | | | LIAIVMVTIMLCCM | | LHVTYVPAQEKN | |
| | | | TSCCSCLKGCCSCG | | FTTAPAICHDGK | |
| | | | SCCKFDEDDSEPVL | | AHFPREGVFVSN | |
| | | | KGVKLHYT | | GTHWFVTQRNFY | |
| | | | | | EPQIITTDNTFV | |
| | | | | | SGNCDVVIGIVN | |
| | | | | | NTVYDPLQPELD | |
| | | | | | SFKEELDKYFKN | |
| | | | | | HTSPDVDLGDIS | |
| | | | | | GINASVVNIQKE | |
| | | | | | IDRLNEVAKNLN | |
| | | | | | ESLIDLQELGKY | |
| | | | | | EQYIKWPWYIWL | |
| | | | | | GFIAGLIAIVMV | |
| | | | | | TIMLCCMTSCCS | |
| | | | | | CLKGCCSCGSCC | |
| | | | | | KFDEDDSEPVLK | |
| | | | | | GVKLHYT | |

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of any SARS-CoV-2 protein set forth in Table 5. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least 1, 2, 3, 4, 5, or 6 of the sets of amino acid substitutions set forth in Table 4, and the other than the at least 1, 2, 3, 4, 5, or 6 of the sets of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any SARS-Cov-2 protein set forth in Table 5.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 29-76. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least 1, 2, 3, 4, 5, or 6 of the sets of amino acid substitutions set forth in Table 4, and the other than the at least 1, 2, 3, 4, 5, or 6 of the sets of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 29-76.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 29-44. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least 1, 2, 3, 4, 5, or 6 of the sets of amino acid substitutions set forth in Table 4, and the other than the at least 1, 2, 3, 4, 5, or 6 of the sets of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 29-44.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 45-76. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least 1, 2, 3, 4, 5, or 6 of the sets of amino acid substitutions set forth in Table 4, and the other than the at least 1, 2, 3, 4, 5, or 6 of the sets of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 45-76.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 45-60. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least 1, 2, 3, 4, 5, or 6 of the sets of amino acid substitutions set forth in Table 4, and the other than the at least 1, 2, 3, 4, 5, or 6 of the sets of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 45-60.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 61-76. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least 1, 2, 3, 4, 5, or 6 of the sets of amino acid substitutions set forth in Table 2, and the other than the at least 1, 2, 3, 4, 5, or 6 of the sets of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 61-76.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least about 10, 15, 20, 30, 40, 50, 60, 70, 80, 90, 100, 150, 200, 300, 400, 500, 600, 700, 800, 900, 1000, 1100, 1200, or 1300 amino acids. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises no more than about 10, 15, 20, 30, 40, 50, 60, 70, 80, 90, 100, 150, 200, 300, 400, 500, 600, 700, 800, 900, 1000, 1100, 1200, or 1300 amino acids. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises from about 10-1300, 10-1200, 10-1100, 10-1000, 10-900, 10-800, 10-700, 10-600, 10-500, 10-400, 10-500, 10-400, 10-300, 10-250, 10-200, 10-100, 10-90, 10-80, 10-70, 10-60, 10-50, 10-40, 10-30, 10-20, 10-1300, 20-1300, 30-1300, 40-1300, 50-1300, 60-1300, 70-1300, 80-1300, 90-1300, 100-1300, 10-250, 20-250, 30-250, 40-250, 50-250, 60-250, 70-250, 80-250, 90-250, or 100-250 amino acids.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of any SARS-CoV-2 protein set forth in Table 5. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of at least 1, 2, 3, 4, 5, or 6 of the sets of amino acid substitutions set forth in Table 4, and the other than the at least 1, 2, 3, 4, 5, or 6 of the sets of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of any SARS-Cov-2 protein set forth in Table 5.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 29-76. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of at least 1, 2, 3, 4, 5, or 6 of the sets of amino acid substitutions set forth in Table 4, and the other than the at least 1, 2, 3, 4, 5, or 6 of the sets of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 29-76.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 29-44. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of at least 1, 2, 3, 4, 5, or 6 of the sets of amino acid substitutions set forth in Table 4, and the other than the at least 1, 2, 3, 4, 5, or 6 of the sets of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 29-44.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 45-76. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of at least 1, 2, 3, 4, 5, or 6 of the sets of amino acid substitutions set forth in Table 4, and the other than the at least 1, 2, 3, 4, 5, or 6 of the sets of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 45-76.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 45-60. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of at least 1, 2, 3, 4, 5, or 6 of the sets of amino acid substitutions set forth in Table 4, and the other than the at least 1, 2, 3, 4, 5, or 6 of the sets of amino acid substitutions set forth in Table 4, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 45-60.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 61-76. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of at least 1, 2, 3, 4, 5, or 6 of the sets of amino acid substitutions set forth in Table 2, and the other than the at least 1, 2, 3, 4, 5, or 6 of the sets of amino acid substitutions set forth in Table 2, the amino acid sequence of the SARS-CoV-2 spike protein or polypeptide (e.g., the SARS-CoV-2 spike protein or polypeptide immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 61-76.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of at least about 10, 15, 20, 30, 40, 50, 60, 70, 80, 90, 100, 150, 200, 300, 400, 500, 600, 700, 800, 900, 1000, 1100, 1200, or 1300 amino acids. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of no more than about 10, 15, 20, 30, 40, 50, 60, 70, 80, 90, 100, 150, 200, 300, 400, 500, 600, 700, 800, 900, 1000, 1100, 1200, or 1300 amino acids. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) consists of from about 10-1300, 10-1200, 10-1100, 10-1000, 10-900, 10-800, 10-700, 10-600, 10-500, 10-400, 10-500, 10-400, 10-300, 10-250, 10-200, 10-100, 10-90, 10-80, 10-70, 10-60, 10-50, 10-40, 10-30, 10-20, 10-1300, 20-1300, 30-1300, 40-1300, 50-1300, 60-1300, 70-1300, 80-1300, 90-1300, 100-1300, 10-250, 20-250, 30-250, 40-250, 50-250, 60-250, 70-250, 80-250, 90-250, or 100-250 amino acids.

### 5.2.1 Exemplary Additional Features of SARS-CoV-2 Spike Proteins (e.g., Immunogens)

The SARS-CoV-2 spike protein or polypeptide (e.g., the SARS-CoV-2 spike protein or polypeptide immunogen (or immunogenic fragment and/or immunogenic variant thereof)) can exhibit additional structural and functional features. Exemplary additional structural and functional features are provided below.

### 5.2.1.1 Full-Length Spike Proteins and Receptor Binding Fragments

In some embodiments, the SARS-CoV-2 spike protein or polypeptide (e.g., the SARS-CoV-2 spike protein or polypeptide immunogen (or immunogenic fragment and/or immunogenic variant thereof)) comprises the full-length SARS-CoV-2 spike protein. In some embodiments, the SARS-CoV-2 spike protein or polypeptide (e.g., the SARS-CoV-2 spike protein or polypeptide immunogen (or immunogenic fragment and/or immunogenic variant thereof)) consists of the full-length SARS-CoV-2 spike protein.

In some embodiments, the SARS-CoV-2 spike protein or polypeptide (e.g., the SARS-CoV-2 spike protein or polypeptide immunogen (or immunogenic fragment and/or immunogenic variant thereof)) comprises a fragment of the full-length SARS-CoV-2 spike protein that is capable of binding the ACE2 receptor. In some embodiments, the SARS-CoV-2 spike protein or polypeptide (e.g., the SARS-CoV-2 spike protein or polypeptide immunogen (or immunogenic fragment and/or immunogenic variant thereof)) consists of a fragment of the full-length SARS-CoV-2 spike protein that is capable of binding the ACE2 receptor.

In some embodiments, the SARS-CoV-2 spike protein or polypeptide (e.g., the SARS-CoV-2 spike protein or polypeptide immunogen (or immunogenic fragment and/or immunogenic variant thereof)) comprises at least a portion of a RBD of the SARS-CoV-2 spike protein. In some embodiments, the SARS-CoV-2 spike protein or polypeptide (e.g., the SARS-CoV-2 spike protein or polypeptide immunogen (or immunogenic fragment and/or immunogenic variant thereof)) comprises a RBD of the SARS-CoV-2 spike protein. In some embodiments, the SARS-CoV-2 spike protein or polypeptide (e.g., the SARS-CoV-2 spike protein or polypeptide immunogen (or immunogenic fragment and/or immunogenic variant thereof)) consists of at least a portion of a RBD of the SARS-CoV-2 spike protein. In some embodiments, the SARS-CoV-2 spike protein or polypeptide (e.g., the SARS-CoV-2 spike protein or polypeptide immunogen (or immunogenic fragment and/or immunogenic variant thereof)) consists of a RBD of the SARS-CoV-2 spike protein.

In some embodiments, the SARS-CoV-2 spike protein or polypeptide (e.g., the SARS-CoV-2 spike protein or polypeptide immunogen (or immunogenic fragment and/or immunogenic variant thereof)) comprises at least a portion of the RBD of the SARS-CoV-2 spike protein. In some embodiments, the SARS-CoV-2 spike protein or polypeptide (e.g., the SARS-CoV-2 spike protein or polypeptide immunogen (or immunogenic fragment and/or immunogenic variant thereof)) comprises the RBD of the SARS-CoV-2 spike protein. In some embodiments, the SARS-CoV-2 spike protein or polypeptide (e.g., the SARS-CoV-2 spike protein or polypeptide immunogen (or immunogenic fragment and/or immunogenic variant thereof)) consists of at least a portion of the RBD of the SARS-CoV-2 spike protein. In some embodiments, the SARS-CoV-2 spike protein or polypeptide (e.g., the SARS-CoV-2 spike protein or polypeptide immunogen (or immunogenic fragment and/or immunogenic variant thereof)) consists of the RBD of the SARS-CoV-2 spike protein.

In some embodiments, the SARS-CoV-2 spike protein or polypeptide (e.g., the SARS-CoV-2 spike protein or polypeptide immunogen (or immunogenic fragment and/or immunogenic variant thereof)) comprises the ectodomain of the SARS-CoV-2 spike protein. In some embodiments, the SARS-CoV-2 spike protein or polypeptide (e.g., the SARS-CoV-2 spike protein or polypeptide immunogen (or immunogenic fragment and/or immunogenic variant thereof)) comprises the ectodomain of the SARS-CoV-2 spike protein.

In some embodiments, the SARS-CoV-2 spike protein or polypeptide (e.g., the SARS-CoV-2 spike protein or polypeptide immunogen (or immunogenic fragment and/or immunogenic variant thereof)) does not include the transmembrane domain of the SARS-CoV-2 spike protein. In some embodiments, the SARS-CoV-2 spike protein or polypeptide (e.g., the SARS-CoV-2 spike protein or polypeptide immunogen (or immunogenic fragment and/or immunogenic variant thereof)) does not include the cytoplasmic domain of the SARS-CoV-2 spike protein. In some embodiments, the SARS-CoV-2 spike protein or polypeptide (e.g., the SARS-CoV-2 spike protein or polypeptide immunogen (or immunogenic fragment and/or immunogenic variant thereof)) does not include the transmembrane domain or the cytoplasmic domain of the SARS-CoV-2 spike protein.

### 5.2.1.2 Stabilization

In some embodiments, the SARS-CoV-2 spike protein or polypeptide (e.g., the SARS-CoV-2 spike protein or polypeptide immunogen (or immunogenic fragment and/or immunogenic variant thereof)) is modified to improve stability of the protein produced. In some embodiments, the ectodomain of the SARS-CoV-2 spike protein or polypeptide (e.g., the SARS-CoV-2 spike protein or polypeptide immunogen (or immunogenic fragment and/or immunogenic variant thereof)) is modified to improve stability of the protein produced.

In some embodiments, the SARS-CoV-2 spike protein or polypeptide (e.g., the SARS-CoV-2 spike protein or polypeptide immunogen (or immunogenic fragment and/or immunogenic variant thereof)) is stabilized in a prefusion state. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least one amino acid variation that stabilizes said SARS-CoV-2 spike protein (e.g., the SARS-CoV-2 spike protein immunogen (or the immunogenic fragment and/or immunogenic variant thereof)) in a prefusion state. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least one amino acid variation relative to the amino acid sequence set forth in any one of SEQ ID NO: 1 that stabilizes the SARS-CoV-2 spike protein or polypeptide (e.g., the SARS-CoV-2 spike protein or polypeptide immunogen (or immunogenic fragment and/or immunogenic variant thereof)) in a prefusion state.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises a proline at one or more of position 817, 892, 899, 986, and/or 987, amino acid numbering relative to the amino acid positions set forth in SEQ ID NO: 1. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises a proline at positions 817, 892, 899, 986, and 987, amino acid numbering relative to the amino acid positions set forth in SEQ ID NO: 1.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least one amino acid variation relative to the amino acid sequence set forth in SEQ ID NO: 1, SEQ ID NO: 2, SEQ ID NO: 6, or SEQ ID NO: 7 that stabilizes the SARS-CoV-2 spike protein or polypeptide (e.g., the SARS-CoV-2 spike protein or polypeptide immunogen (or immunogenic fragment and/or immunogenic variant thereof)) in a prefusion state. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises a proline at amino acid position 986 and/or a proline at amino acid position 987, amino acid numbering amino acid numbering relative to SEQ ID NO: 1. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises a proline at amino acid position 986 and/or a proline at amino acid position 987, amino acid numbering relative to SEQ ID NO: 1.

In some embodiments, the SARS-CoV-2 spike protein or polypeptide (e.g., the SARS-CoV-2 spike protein or polypeptide immunogen (or immunogenic fragment and/or immunogenic variant thereof)) comprises an inactive furin cleavage site. In order to be able to trigger fusion, the spike protein of SARS-Cov-2 has to be cleaved into the S1 and S2 subunit. The cleavage site in SARS-Cov-2 is a polybasic motif (RRAR) (for example, amino acids 682-685 of SEQ ID NO: 1) that can be activated by furin-like proteases. Modifications to the furin cleavage site that inactivate it are known in the art. See, e.g., Amanat F, Strohmeier S, Rathnasinghe R, et al. Introduction of two prolines and removal of the polybasic cleavage site leads to optimal efficacy of a recombinant spike-based SARS-CoV-2 vaccine in the mouse model. Preprint. bioRxiv. 2020; 2020.09.16.300970. Published 2020 Sep. 17. doi:10.1101/2020.09.16.300970 (describes the replacement of the RRAR cleavage site with a single alanine); and WO2022203963 (which describes the replacement of the RRAR cleavage site with the amino acid sequence QQAQ), the entire contents of each of which are incorporated by reference herein. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least one amino acid variation in the furin cleavage site that inactivates the furin cleavage site. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises a replacement of the RRAR cleavage site with a single alanine. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises a replacement of the RRAR cleavage site with the amino acid sequence QQAQ.

### 5.2.1.3 Multimerization (e.g., Trimerization) Domains

In some embodiments, the SARS-CoV-2 spike protein or polypeptide (e.g., the SARS-CoV-2 spike protein or polypeptide immunogen (or immunogenic fragment and/or immunogenic variant thereof)) comprises a multimerization (e.g., trimerization) domain. The multimerization domain (e.g., trimerization domain) facilities multimerization (e.g., trimerization) of the proteins upon expression in/from a cell. Suitable multimerization (e.g., trimerization) domains are known in the art, including e.g., a fibritin—based trimerization domain. This fibritin domain or ‘Foldon’ is derived from T4 fibritin and was previously described as an artificial natural trimerization domain. See, e.g., (Letarov et al., 1993) Biochemistry Moscow 64: 817-823; S-Guthe et al., (2004) J. Mol. Biol. 337: 905-915). See, also, Meier et al., J Mol Biol. (2004) 344(4): 1051-69; WO 2018/081318; the entire contents of each of which are incorporated herein by reference for all purposes.

### 5.2.1.4 Engineered Glycosylation Sites

In some embodiments, the SARS-CoV-2 spike protein or polypeptide (e.g., the SARS-CoV-2 spike protein or polypeptide immunogen (or immunogenic fragment and/or immunogenic variant thereof)) comprises one or more non-naturally occurring glycosylation motif comprising a non-naturally occurring glycosylation site (e.g., compared to a reference protein). See, e.g., Lin Wei-Shuo et al., Glycan Masking of Epitopes in the NTD and RBD of the Spike Protein Elicits Broadly Neutralizing Antibodies Against SARS-CoV-2 Variants, Frontiers in Immunology, (12) Article 795741, 2 Dec. 2021, DOI=10.3389/fimmu.2021.795741, the entire contents of which are incorporated herein by reference for all purposes.

In some embodiments, the inclusion of one or more non-naturally occurring glycosylation motif (e.g., N-glycosylation motif) comprising a non-naturally occurring glycosylation site facilitates glycan masking of an immunodominant epitope of the SARS-CoV-2 spike protein or polypeptide (e.g., the SARS-CoV-2 spike protein or polypeptide immunogen (or immunogenic fragment and/or immunogenic variant thereof)). In some embodiments, when the SARS-CoV-2 spike protein or polypeptide (e.g., the SARS-CoV-2 spike protein or polypeptide immunogen (or immunogenic fragment and/or immunogenic variant thereof)) is administered to a subject, the subject does not generate an effective number of neutralizing antibodies that specifically bind to the immunodominant epitope. Thus, in some embodiments, the inclusion of one or more non-naturally occurring glycosylation motif (e.g., N-glycosylation motif) comprising a non-naturally occurring glycosylation site shifts an immune response generated from the administration of the SARS-CoV-2 spike protein or polypeptide (e.g., the SARS-CoV-2 spike protein or polypeptide immunogen (or immunogenic fragment and/or immunogenic variant thereof)) to produce more neutralizing antibodies against the SARS-CoV-2 spike protein or polypeptide (e.g., the SARS-CoV-2 spike protein or polypeptide immunogen (or immunogenic fragment and/or immunogenic variant thereof)).

In some embodiments, the SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site relative to the amino acid sequence set forth in any one of SEQ ID NOS: 1-10. In some embodiments, the SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site relative to the amino acid sequence set forth in SEQ ID NO: 1 In some embodiments, the SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site relative to the amino acid sequence set forth in SEQ ID NO: 2. In some embodiments, the SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site relative to the amino acid sequence set forth in SEQ ID NO: 3. In some embodiments, the SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site relative to the amino acid sequence set forth in SEQ ID NO: 4. In some embodiments, the SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site relative to the amino acid sequence set forth in SEQ ID NO: 5. In some embodiments, the SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site relative to the amino acid sequence set forth in SEQ ID NO: 6. In some embodiments, the SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site relative to the amino acid sequence set forth in SEQ ID NO: 7. In some embodiments, the SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site relative to the amino acid sequence set forth in SEQ ID NO: 8. In some embodiments, the SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site relative to the amino acid sequence set forth in SEQ ID NO: 9. In some embodiments, the SARS-CoV-2 spike protein (e.g., the SARS-CoV-2 spike protein immunogen (or immunogenic fragment and/or immunogenic variant thereof)) comprises one or more non-naturally occurring glycosylation motif comprising a non-naturally occurring glycosylation site relative to the amino acid sequence set forth in SEQ ID NO: 10.

In some embodiments, the non-naturally occurring glycosylation motif is an N-linked glycosylation motif (e.g., NXT or NXS (wherein X is any amino acid except for proline)). In some embodiments, the non-naturally occurring glycosylation motif is an O-linked glycosylation motif (e.g., a serine or threonine). In some embodiments, the non-naturally occurring glycosylation site is an N-linked glycosylation site (an asparagine (N) residue within a N-linked glycosylation motif (e.g., NXT or NXS (wherein X is any amino acid except for proline)). In some embodiments, the non-naturally occurring glycosylation site is an O-linked glycosylation site (e.g., a serine or threonine). Glycosylation motifs are known in the art. For example, NX1X2, wherein X1 can be any amino acid except for proline, and X2 can be S, T, or C, is known as a consensus N-glycosylation sequence. O-linked glycosylation motifs include single serine or threonine residues.

### 5.2.1.5 Hyperglycosylation

In some embodiments, the SARS-CoV-2 spike protein (e.g., the SARS-CoV-2 spike protein immunogen (or an immunogenic fragment and/or immunogenic variant thereof)) is hyper-glycosylated relative to a reference SARS-CoV-2 spike protein (e.g., the SARS-CoV-2 spike protein immunogen (or an immunogenic fragment and/or immunogenic variant thereof)) (e.g., the SARS-CoV-2 spike protein set forth in SEQ ID NO: 1) (e.g., a SARS-CoV-2 spike protein that does not contain the one or more non-naturally occurring glycosylation sites).

Standard methods known in the art can be utilized to evaluate the glycosylation of a protein or peptide, including e.g., the level of glycosylation, the sites of glycosylation, etc. See, e.g., Roth et al, Identification and Quantification of Protein Glycosylation (2012) International Journal of Carbohydrate Chemistry, Volume 201, Article ID 640923, https://doi.org/10.1155/2012/640923; Lebede et al, Exploring the Chemical Space of Protein Glycosylation in Noncovalent Protein Complexes: An Expedition along Different Structural Levels of Human Chorionic Gonadotropin by Employing Mass Spectrometry, Anal. Chem. 2021, 93, 30, 10424-10434 (Jul. 21, 2021) https://doi.org/10.1021/acs.analchem.lc02199; Leymarie et al., Effective Use of Mass Spectrometry for Glycan and Glycopeptide Structural Analysis, Anal. Chem. 2012, 84, 7, 3040-3048 (Feb. 21, 2012) https://doi.org/10.1021/ac3000573 the entire contents of each of which is incorporated by reference herein for all purposes. Standard methods include for example, the enzymatic or chemical release of glycans from the subject proteins followed by chromatography or mass spectrometry based structural analysis. Glycan site mapping is further standard in art through the use of enzymatic digestions and subsequent tandem mass spectrometry-based analysis. See, e.g., Roth et al, Identification and Quantification of Protein Glycosylation (2012) International Journal of Carbohydrate Chemistry, Volume 201, Article ID 640923, https://doi.org/10.1155/2012/640923, the entire contents of which is incorporated by reference herein for all purposes.

### 5.2.1.6 Additional Variations

In some embodiments, the SARS-CoV-2 spike protein or polypeptide (e.g., the SARS-CoV-2 spike protein or polypeptide immunogen (or immunogenic fragment and/or immunogenic variant thereof)) comprises an amino acid sequence comprising at least 1, 2, 3, 4, 5, 6, 7, 8, 9, or 10 amino acid variants that are not listed in Table 2 or Table 4. In some embodiments, the SARS-CoV-2 spike protein or polypeptide (e.g., the SARS-CoV-2 spike protein or polypeptide immunogen (or immunogenic fragment and/or immunogenic variant thereof)) comprises an amino acid sequence comprising or consisting of 1, 2, 3, 4, 5, 6, 7, 8, 9, or 10 amino acid variants that are not listed in Table 2 or Table 4. In some embodiments, the amino acid variants not listed in Table 2 or Table 4 are found in one or more circulating variants of SARS-CoV-2.

In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least one set of amino acid substitutions set forth in Table 2 and at least one set of amino acid substitutions set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least 2, 3, 4, 5, or 6 sets of amino acid substitutions set forth in Table 2. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises at least 2, 3, 4, 5, 6, 7, 8, 9, 10, 11, 12, 13, 14, 15, or 16 sets of amino acid substitutions set forth in Table 4. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) that are not listed in Table 2 relative to the amino acid sequence set forth in any one of SEQ ID NOS: 1-5. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 2 relative to the amino acid sequence set forth in any one of SEQ ID NOS: 1-5. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) that are not listed in Table 4 relative to the amino acid sequence set forth in any one of SEQ ID NOS: 1-5. In some embodiments, the amino acid sequence of the SARS-CoV-2 spike protein or polypeptide (e.g., the SARS-CoV-2 spike protein or polypeptide immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%), amino acid variations (e.g., substitutions, additions, deletions, etc.) that are not listed in Table 4 relative to the amino acid sequence set forth in any one of SEQ ID NOS: 1-5. In some embodiments, the amino acid variants not listed in Table 2 or Table 4 are found in one or more circulating variants of SARS-CoV-2.

5.2.1.7 Variations from Different Strains of SARS-CoV-2

In some embodiments, the amino acid sequence of the SARS-CoV-2 spike protein is derived from a circulating strain (i.e., naturally occurring variant) of SARS-CoV-2. In some embodiments, the amino acid sequence of the SARS-CoV-2 spike protein comprises one or more amino acid variation found in one or more circulating variants of SARS-CoV-2. In some embodiments, the amino acid sequence of the SARS-CoV-2 spike protein is derived from a strain of SARS-CoV-2 known to have previously circulated. In some embodiments, the amino acid sequence of the SARS-CoV-2 spike protein comprises one or more amino acid variation found in one or more SARS-CoV-2 variants known to have previously circulated. For example, in some embodiments, the amino acid sequence of the SARS-CoV-2 spike protein comprises a glycine at position 614, amino acid numbering relative to the amino acid positions set forth in SEQ ID NO: 1.

### 5.3 Nucleic Acid Molecules

Provided herein are, inter alia, nucleic acid molecules comprising a coding region encoding a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2). In some embodiments, the nucleic acid molecule is a DNA molecule. In some embodiments, the nucleic acid molecule is an RNA molecule (e.g., mRNA or circular RNA). In some embodiments, the nucleic acid (e.g., RNA) molecule is a translatable RNA. In some embodiments, the nucleic acid (e.g., RNA) molecule is an mRNA. In some embodiments, the nucleic acid (e.g., RNA) molecule is a circular RNA.

In some embodiments, the nucleic acid molecule encoding the SARS-CoV-2 spike protein or polypeptide (e.g., a SARS-CoV-2 spike protein or polypeptide immunogen (or immunogenic fragment and/or immunogenic variant thereof)) (e.g., described herein) comprises from about 30 to about 20000 nucleotides, about 50 to about 20000 nucleotides, about 500 to about 10000 nucleotides, about 1000 to about 10000 nucleotides, about 1000 to about 5000 nucleotides, or about 2000 to about 5000 nucleotides. In some embodiments, the nucleic acid molecule encoding the SARS-CoV-2 spike protein or polypeptide (e.g., a SARS-CoV-2 spike protein or polypeptide immunogen (or immunogenic fragment and/or immunogenic variant thereof)) comprises at least 30 nucleotides, 50 nucleotides, 100 nucleotides, 200 nucleotides, 300 nucleotides, 400 nucleotides, 500 nucleotides, 1000 nucleotides, 2000 nucleotides, 3000 nucleotides, or 5000 nucleotides.

In some embodiments, the nucleic acid molecule is altered (e.g., compared to the sequence of a reference nucleic acid molecule, a naturally occurring nucleic acid molecule), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide modifications (see, e.g., described herein), etc.

In some embodiments, the sequence of the nucleic acid molecule is codon optimized, e.g., for expression in humans. Codon optimization, in some embodiments, may be used to match codon frequencies in target and host organisms to ensure proper folding; bias guanosine (G) and/or cytosine (C) content to increase nucleic acid stability; minimize tandem repeat codons or base runs that may impair gene construction or expression; customize transcriptional and translational control regions; insert or remove protein trafficking sequences; remove/add post translation alteration sites in an encoded protein (e.g., glycosylation sites); add, remove, or shuffle protein domains; insert or delete restriction sites; modify ribosome binding sites and mRNA degradation sites; adjust translational rates to allow the various domains of a protein to fold properly; and/or to reduce or eliminate secondary structures (e.g., problem secondary structures) within the nucleic acid molecule. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms (e.g., optimization algorithms known in the art). In some embodiments, the nucleic acid sequence is modified to optimize the number of G and/or C nucleotides as compared to a reference nucleic acid sequence. An increase in the number of G and C nucleotides may be generated by substitution of codons containing adenosine (A) or thymidine (T) (or uracil (U)) nucleotides by codons containing G or C nucleotides.

### 5.3.1 DNA Molecules

In some embodiments, the nucleic acid molecule is a DNA molecule. In some embodiments, the DNA is a linear coding DNA construct. In some embodiments, the DNA contained within a vector (e.g., a non-viral vector (e.g., a plasmid) or a viral vector). In some embodiments, the DNA is contained within a non-viral vector (e.g., a plasmid). In some embodiments, the DNA is contained within a viral vector (e.g., described herein). A more detailed description of vectors for both RNA and DNA nucleic acids is provided in § 5.6.

The DNA molecule may also comprise one or more heterologous nucleic acid elements to mediate expression of the coding region. These include, e.g., promoter(s), enhancer(s), polyadenylation signal(s), synthetic introns, transcriptional termination signals, polyadenylation sequences, and other transcription regulatory elements. A person of ordinary skill in the art is familiar with the transcriptional regulatory elements needed for expression of coding DNA can optimize the expression construct (e.g., linear DNA, plasmid DNA, etc.) accordingly.

In some embodiments, a promoter is operably connected to the respective coding nucleic acid sequence. The person of ordinary skill in the art is aware of various promoters that can be employed, for example, a promoter from simian virus 40 (SV40), a mouse mammary tumor virus (MMTV) promoter, a human immunodeficiency virus (HIV) promoter, bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene, for example, from human actin, human myosin, human hemoglobin, human muscle creatine, or human metalothionein. The promoter can also be a tissue specific promoter, such as a muscle or skin specific promoter, natural or synthetic. Examples of such promoters are described in US Patent Application Publication No. US20040175727, the entire contents of which is incorporated by reference herein for all purposes. Exemplary polyadenylation signals, include, but are not limited, to the bovine growth hormone (BGH) polyadenylation site, SV40 polyadenylation signals, and LTR polyadenylation signals.

### 5.3.2 RNA Molecules

In some embodiments, the nucleic acid molecule is an RNA molecule. In some embodiments, the RNA molecule is a translatable RNA molecule. In some embodiments, the RNA molecule is selected from an mRNA, a self-replicating RNA, a circular RNA, a viral RNA, or a replicon RNA.

In some embodiments, the RNA molecule a circular RNA. Exemplary circular RNAs are described in e.g., U.S. Ser. No. 11/458,156, US20220143062, US20230212629, US20230072532, U.S. Ser. No. 11/203,767, U.S. Ser. No. 11/352,641, US20210371494, U.S. Ser. No. 11/766,449, US20230226096, WO2021189059, US20190345503, US20220288176, U.S. Ser. No. 11/560,567, WO2022271965, WO2022037692, WO2023024500, WO2023115732, WO2023133684, WO2023143541, WO2023134611, and WO2022247943, the entire contents of each of which are incorporated herein by reference for all purposes.

In some embodiments, the RNA molecule is a mRNA. The basic components of an mRNA molecule typically include at least one coding region (herein a coding region encoding at least one peptide or protein (e.g., a SARS-CoV-2 protein (e.g., a SARS-CoV-2 spike protein or polypeptide immunogen (or an immunogenic fragment and/or variant thereof)) (e.g., described herein), a 5′-untranslated region (UTR), a 3′-UTR, a 5′ cap, and a poly-A tail.

In some embodiments, the RNA (e.g., mRNA) comprises at least one heterologous UTR. The UTRs may harbor regulatory sequence elements that determine the RNA (e.g., mRNA) turnover, stability, localization, and/or expression of operably connected coding sequence(s). The heterologous UTRs may be derived from a naturally occurring gene or may be synthetically engineered. In some embodiments, the 5′-UTR comprises elements for controlling gene expression, e.g., ribosomal binding sites, miRNA binding sites. The 5′-UTR may be post-transcriptionally modified, e.g., by enzymatic or post-transcriptional addition of a 5′cap structure. In some embodiments, the 3′-UTR comprises a polyadenylation signal. In some embodiments, the RNA (e.g., mRNA) comprises at least one coding region encoding the polypeptide or protein (e.g., a SARS-CoV-2 protein (e.g., a SARS-CoV-2 spike protein or polypeptide immunogen (or an immunogenic fragment and/or immunogenic variant thereof)) (e.g., described herein) and 5′-UTR and/or a 3′-UTR. In some embodiments, the RNA (e.g., mRNA) comprises at least one coding sequence encoding a polypeptide or protein (e.g., a SARS-CoV-2 protein (e.g., a SARS-CoV-2 spike protein or polypeptide immunogen (or an immunogenic fragment and/or immunogenic variant thereof)) (e.g., described herein) operably connected to at least one heterologous 5′-UTR and at least one 3′-UTR.

In some embodiments, the RNA (e.g., mRNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides. In some embodiments, poly(A) sequence comprises at least 10, 20, 30, 40, 50, 60, 70, 80, 90, 100, 200, 300, 400, or 500 adenosine nucleotides. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides, wherein the 3′ terminal nucleotide of said nucleic acid molecule is an adenosine. In some embodiments, poly(A) sequence comprises at least 10, 20, 30, 40, 50, 60, 70, 80, 90, 100, 200, 300, 400, or 500 adenosine nucleotides, wherein the 3′ terminal nucleotide of said nucleic acid molecule is an adenosine.

In some embodiments, the RNA (e.g., mRNA) comprises a 5′-cap structure. In some embodiments, the 5′-cap structure stabilizes the RNA (e.g., mRNA), enhances expression of the encoded polypeptide or protein (e.g., a SARS-CoV-2 protein (e.g., a SARS-CoV-2 spike protein or polypeptide immunogen (or an immunogenic fragment and/or immunogenic variant thereof)) (e.g., described herein) and/or reduces the stimulation of the innate immune system (e.g., after administration to a subject).

Exemplary 5′-cap structures include, but are not limited to, cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g., phosphorothioate modified ARCA), inosine, N1-methyi-guanosine, 2′-fluoro-guanosine, 7-deaza-guanosine, 8-oxo-guanosine, 2-amino-guanosine, LNA-guanosine, and 2-azido-guanosine. In some embodiments, the 5′ cap structure comprises m7G, cap0, cap1, cap2, a modified capO, or a modified cap1 structure.

In some embodiments, the RNA (e.g., mRNA) comprises one or more modified nucleotide (e.g., nucleotide analogue, backbone modifications, sugar modifications, and/or base modifications). A backbone modification in the context of the present disclosure is a modification, in which phosphates of the backbone of the nucleotides of the RNA (e.g., mRNA) are chemically modified. A sugar modification in the context of the present disclosure is a chemical modification of the sugar of the nucleotides of the RNA (e.g., mRNA). A base modification in the context of the present disclosure is a chemical modification of the base moiety of the nucleotides of the RNA (e.g., mRNA).

In some embodiments, the RNA (e.g., mRNA) comprises at least one modified nucleotide. Exemplary nucleotide analogues/modifications include, but are not limited to, 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thiocytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphatej-uridine, 5′-O-(1-thiophosphate)-pseudouridine, 6-aza-cytidine, 2-thio-cytidine, alpha-thio-cytidine, Pseudoiso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thioguanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thioadenosine, 8-azido-adenosine, and 7-deaza-adenosine.

In some embodiments, the RNA (e.g., mRNA) comprises pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and/or 2′-O-methyl uridine.

In some embodiments, the RNA (e.g., mRNA) comprises one or more pseudouridine (ψ), N1-methylpseudouridine (mlψ), 5-methylcytosine, and 5-methoxyuridine. In some embodiments, essentially all, e.g., essentially 100% of the uracil in the coding sequence of the RNA (e.g., mRNA) have a chemical modification, preferably a chemical modification is in the 5-position of the uracil. Incorporating modified nucleotides such as e.g., pseudouridine (ψ), N1-methylpseudouridine (mlψ), 5-methylcytosine, and/or 5-methoxyuridine into the coding sequence may be advantageous as unwanted innate immune responses (upon administration of the coding RNA or the vaccine) may be adjusted or reduced (if required).

In one embodiment, the RNA (e.g., mRNA) comprises: (i) a 5′-cap structure; (ii) a 5′-UTR; (iii) N1-methyl-pseudouridine, cytosine, adenine, and guanine; (iv) a 3′-UTR; and (v) a poly-A region.

RNA (e.g., mRNA) described herein can be generated by e.g., in vitro transcription. In vitro transcription is a method well known to those of ordinary skill in the art for the production of RNA (e.g., mRNA). Generally, the RNA is obtained by DNA-dependent in vitro transcription of an appropriate DNA template, e.g., a linearized plasmid DNA template or a PCR-amplified DNA template. The promoter for controlling RNA in vitro transcription can be any promoter for any DNA-dependent RNA polymerase. Examples of DNA-dependent RNA polymerases include the 17, T3, SP6, or Syn5 RNA polymerases. In some instances, the DNA template is linearized with a suitable restriction enzyme before it is subjected to RNA in vitro transcription. Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); a DNA-dependent RNA polymerase capable of binding to the promoter sequence within the DNA template (e.g., T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCh, which supplies Mg2+ ions as a co-factor for the polymerase; a buffer (TRIS or HEPES) to maintain a suitable pH value, which can also contain antioxidants (e.g., DTT), and/or polyamines such as spermidine at optimal concentrations, e.g., a buffer system comprising TRIS-Citrate as disclosed in WO2017109161. The obtained RNA (e.g., mRNA) products can be purified according to methods known in the art. For example, using PureMessenger® (CureVac, Tubingen, Germany; RP-HPLC according to WO2008077592) and/or tangential flow filtration (as described in WO2016193206) and/or oligo d(T) purification (see WO2016180430); or using RP-HPLC, e.g., using Reversed-Phase High pressure liquid chromatography (RP-HPLC), the entire contents of each reference is incorporated by reference herein for all purposes.

### 5.4 Fusions & Conjugates

In some embodiments, the SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2) or the nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.3) is operably connected to a heterologous moiety (e.g., a heterologous polypeptide) forming a fusion protein or polypeptide or conjugate.

In some embodiments, the heterologous moiety (e.g., a heterologous polypeptide) imparts an additional function to the SARS-CoV-2 spike protein or polypeptide (e.g., the SARS-CoV-2 spike protein or polypeptide immunogen (or immunogenic fragment and/or immunogenic variant thereof)). For example, a heterologous moiety (e.g., a heterologous polypeptide) may function to promote or improve secretion of the encoded SARS-CoV-2 spike protein or polypeptide (e.g., the SARS-CoV-2 spike protein or polypeptide immunogen (or immunogenic fragment and/or immunogenic variant thereof)), (e.g., via secretory signal peptides); promote or improve anchoring of the encoded SARS-CoV-2 spike protein or polypeptide (e.g., the SARS-CoV-2 spike protein or polypeptide immunogen (or immunogenic fragment and/or immunogenic variant thereof)) described herein in the plasma membrane (e.g., via transmembrane elements); promote or improve formation of antigen complexes (e.g., via multimerization domains or antigen clustering elements); and/or promote or improve virus-like particle formation (VLP forming sequence); improve half-life of the SARS-CoV-2 spike protein or polypeptide (e.g., the SARS-CoV-2 spike protein or polypeptide immunogen (or immunogenic fragment and/or immunogenic variant thereof)).

In some embodiments, the heterologous moiety is a half-life extension moiety. Exemplary half-life extension moieties include, but are not limited to, an immunoglobulins (e.g., immunoglobulin constant regions and fragment thereof) (e.g., human immunoglobulin (hIg), a fragment of a hIg, a hIg constant region, a fragment of a hIg constant region, a hIg Fc region), human transferrin, human serum albumin (HSA), an HSA binding protein, and polyethylene glycol (PEG) (and polymers thereof). In some embodiments, the heterologous polypeptide is a half-life extension polypeptide. Exemplary half-life extension polypeptides include, but are not limited to, an immunoglobulins (e.g., immunoglobulin constant regions and fragment thereof) (e.g., human immunoglobulin (hIg), a fragment of a hIg, a hIg constant region, a fragment of a hIg constant region, a hIg Fc region), human transferrin, human serum albumin (HSA), and an HSA binding protein. The SARS-CoV-2 spike protein or polypeptide (e.g., a SARS-CoV-2 spike protein or polypeptide immunogen (or immunogenic fragment and/or immunogenic variant thereof)) or a nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) fused or conjugated to a half-life extending moiety (e.g., a half-life extension polypeptide) can be evaluated for their pharmacokinetic properties utilizing standard in vivo methods known in the art. In some embodiments, the heterologous moiety is a detectable agent (e.g., protein, e.g., a fluorescent protein).

In some embodiments, the heterologous moiety (e.g., heterologous polypeptide) is a signal peptide. In some embodiments, the SARS-CoV-2 spike protein or polypeptide (e.g., the SARS-CoV-2 spike protein or polypeptide immunogen (or immunogenic fragment and/or immunogenic variant thereof)) comprises a homologous signal peptide. In some embodiments, the SARS-CoV-2 spike protein or polypeptide (e.g., the SARS-CoV-2 spike protein or polypeptide immunogen (or immunogenic fragment and/or immunogenic variant thereof)) comprises a heterologous signal peptide. In some embodiments, the SARS-CoV-2 spike protein or polypeptide (e.g., the SARS-CoV-2 spike protein or polypeptide immunogen (or immunogenic fragment and/or immunogenic variant thereof)) does not contain a signal peptide.

In some embodiments, the heterologous moiety (e.g., heterologous polypeptide) is a multimerization element (e.g., trimerization element) (e.g., described herein).

The heterologous moiety (e.g., heterologous polypeptide) can be directly operably connected or indirectly operably connected to the SARS-CoV-2 spike protein or polypeptide (e.g., a SARS-CoV-2 spike protein or polypeptide immunogen (or immunogenic fragment and/or immunogenic variant thereof)) or the nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)).

In some embodiments, the heterologous moiety is directly operably connected to the SARS-CoV-2 spike protein or polypeptide (e.g., a SARS-CoV-2 spike protein or polypeptide immunogen (or immunogenic fragment and/or immunogenic variant thereof)) or the nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)). In some embodiments, the heterologous moiety is indirectly operably connected to the SARS-CoV-2 spike protein or polypeptide (e.g., a SARS-CoV-2 spike protein or polypeptide immunogen (or immunogenic fragment and/or immunogenic variant thereof)) or the nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)).

A person of ordinary skill in the art can evaluate and select a suitable linker for the fusion or conjugation of a specific heterologous moiety. For example, in embodiments, wherein the heterologous moiety comprises a heterologous polypeptide, a peptide linker may be employed. Peptide linkers are known in the art and can be selected based on specific properties, including e.g., length, flexibility, rigidity, cleavability, etc. The amino acid sequence of commonly employed peptide linkers comprises glycine amino acid residues, serine amino acid residues, glycine and serine amino acid residues, or glycine, serine, and proline amino acid residues.

The heterologous moiety (e.g., heterologous polypeptide) and the SARS-CoV-2 spike protein or polypeptide (e.g., a SARS-CoV-2 spike protein or polypeptide immunogen (or immunogenic fragment and/or immunogenic variant thereof)) or the nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) can be arranged in orientation so long as each functional part of the fusion protein or polypeptide or conjugate maintains the ability to mediate its function.

### 5.5 Vaccine Compositions

In some embodiments, a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2) (or a fusion or conjugate thereof (see, e.g., § 5.4)) or a nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.3) (or a fusion or conjugate thereof (see, e.g., § 5.4)) forms the basis for a vaccine composition (e.g., a prime vaccine composition, a vaccine booster composition, a vaccine prime and booster composition). Therefore, provided herein are, inter alia, vaccine compositions (e.g., prime vaccine compositions, vaccine booster compositions, vaccine prime and booster compositions) comprising at least one SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2) (or a fusion or conjugate thereof (see, e.g., § 5.4)) or a nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.3) (or a fusion or conjugate thereof (see, e.g., § 5.4)).

In some embodiments, the vaccine composition is a prime vaccine composition of a prime-boost vaccine regimen. In some embodiments, the vaccine composition is a vaccine booster composition of a prime-boost vaccine regimen. In some embodiments, the vaccine composition is a vaccine prime and booster composition of a prime-boost vaccine regimen. In some embodiments, the vaccine composition can be utilized as a prime and/or a booster (e.g., as described herein). In some embodiments, the vaccine composition forms a single dose vaccine that does not require a booster.

### 5.5.1 Peptide and Protein-Based Vaccines

In some embodiments, a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2) (or a fusion or conjugate thereof (see, e.g., § 5.4)) forms the basis for a vaccine composition (e.g., a prime vaccine composition, a vaccine boost composition, a vaccine prime and booster composition). Therefore, provided herein are, inter alia, vaccine compositions (e.g., prime vaccine compositions, vaccine boost compositions, vaccine prime and booster compositions) comprising at least one SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2) (or a fusion or conjugate thereof (see, e.g., § 5.4)).

In some embodiments, the vaccine composition is a prime vaccine composition of a prime-boost vaccine regimen. In some embodiments, the vaccine composition is a vaccine boost composition of a prime-boost vaccine regimen. In some embodiments, the vaccine composition is a vaccine prime and booster composition of a prime-boost vaccine regimen. In some embodiments, the prime boost composition can be utilized as a prime and/or a booster (e.g., as described herein). In some embodiments, the vaccine composition forms a single dose vaccine that does not require a booster.

In some embodiments, the vaccine composition comprises a plurality of SARS-CoV-2 spike proteins or polypeptides (e.g., immunogens (or immunogenic fragments and/or immunogenic variants thereof)).

In some embodiments, the plurality comprises at least 2, 3, 4, 5, 6, 7, 8, 9, 10, 11, 12, 13, 14, 15, 16, 17, 18, 19, 20, 30, 40, 50, 60, 70, 80, 90, 100 or more SARS-CoV-2 spike proteins or polypeptides (e.g., immunogens (or immunogenic fragments and/or immunogenic variants thereof)). In some embodiments, the plurality comprises from about 2-100, 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 SARS-CoV-2 spike proteins or polypeptides (e.g., immunogens (or immunogenic fragments and/or immunogenic variants thereof)). In some embodiments, the plurality comprises at least 2 but no more than 100, 90, 80, 70, 60, 50, 40, 30, 20, 19, 18, 17, 16, 15, 14, 13, 12, 11, 10, 9, 8, 7, 6, 5, 4, 3 SARS-CoV-2 spike proteins or polypeptides (e.g., immunogens (or immunogenic fragments or immunogenic variants thereof)).

In some embodiments, the amino acid sequence of each of the SARS-CoV-2 spike proteins or polypeptides (e.g., immunogens (or immunogenic fragments and/or immunogenic variants thereof)) of the plurality is different.

In some embodiments, the amino acid sequence of at least one of the SARS-CoV-2 spike proteins or polypeptides (e.g., immunogens (or immunogenic fragments and/or immunogenic variants thereof)) of the plurality is derived from a circulating strain of SARS-CoV-2.

In some embodiments, the plurality comprises at least one SARS-CoV-2 spike protein or polypeptide (e.g., immunogen (or immunogenic fragment and/or immunogenic variant thereof)) described herein. In some embodiments, the plurality comprises at least one SARS-CoV-2 spike protein or polypeptide (e.g., immunogen (or immunogenic fragment and/or immunogenic variant thereof)) described herein, wherein the amino acid sequence of the at least one SARS-CoV-2 spike protein or polypeptide (e.g., immunogen (or immunogenic fragment and/or immunogenic variant thereof)) comprises a set of amino acid substitutions set forth in Table 2 or Table 4.

In some embodiments, the plurality comprises at least two SARS-CoV-2 spike proteins or polypeptides (e.g., immunogens (or immunogenic fragments and/or immunogenic variants thereof)) described herein, wherein the amino acid sequence of each of the at least two SARS-CoV-2 spike proteins or polypeptides (e.g., immunogens (or immunogenic fragments and/or immunogenic variants thereof)) comprises a different set of amino acid substitutions set forth in Table 2 or Table 4 or a different combination of sets of amino acid substitutions set forth in Table 2 or Table 4.

In some embodiments, the amino acid sequence of each of the SARS-CoV-2 spike proteins or polypeptides (e.g., immunogens (or immunogenic fragments and/or immunogenic variants thereof)) of the plurality comprises a different set of amino acid substitutions described herein (e.g., in Table 2 or Table 4) or a different combination of sets of amino acid substitutions described herein (e.g., in Table 2 or Table 4).

In some embodiments, the plurality comprises (a) at least one SARS-CoV-2 spike protein or polypeptide (e.g., immunogen (or immunogenic fragment and/or immunogenic variant thereof)), wherein the amino acid sequence of the at least one SARS-CoV-2 spike protein or polypeptide (e.g., immunogen (or immunogenic fragment and/or immunogenic variant thereof)) comprises at least one set of amino acid substitutions set forth in Table 2; (b) at least one SARS-CoV-2 spike protein or polypeptide (e.g., immunogen (or immunogenic fragment and/or immunogenic variant thereof)), wherein the amino acid sequence of the at least one SARS-CoV-2 spike protein or polypeptide (e.g., immunogen (or immunogenic fragment and/or immunogenic variant thereof)) comprises at least one set of amino acid substitutions set forth in Table 4; and/or (c) at least one SARS-CoV-2 spike protein or polypeptide (e.g., immunogen (or immunogenic fragment and/or immunogenic variant thereof)), wherein the amino acid sequence of the at least one SARS-CoV-2 spike protein or polypeptide (e.g., immunogen (or immunogenic fragment and/or immunogenic variant thereof)) comprises at least one set of amino acid substitutions set forth in Table 2 and at least one set of amino acid substitutions set forth in Table 4.

In some embodiments, plurality comprises at least 2, 3, 4, 5, or 6 SARS-CoV-2 spike proteins or polypeptides (e.g., immunogens (or immunogenic fragments and/or immunogenic variants thereof)), wherein the amino acid sequence of each of said 2, 3, 4, 5, or 6 SARS-CoV-2 spike proteins or polypeptides (e.g., immunogens (or immunogenic fragments and/or immunogenic variants thereof)) comprises a different set of amino acid substitutions set forth in Table 2 or a different combination of sets of amino acid substitutions set forth in Table 2.

In some embodiments, the plurality comprises at least 2, 3, 4, 5, 6, 7, 8, 9, 10, 11, 12, 13, 14, 15, or 16 SARS-CoV-2 spike proteins or polypeptides (e.g., immunogens (or immunogenic fragments and/or immunogenic variants thereof)), wherein the amino acid sequence of each of the 2, 3, 4, 5, 6, 7, 8, 9, 10, 11, 12, 13, 14, 15, or 16 SARS-CoV-2 spike proteins or polypeptides (e.g., immunogens (or immunogenic fragments and/or immunogenic variants thereof)) comprises a different set of amino acid substitutions set forth in Table 4 or a different combination of sets of amino acid substitutions set forth in Table 4.

In some embodiments, plurality comprises (a) at least one SARS-CoV-2 spike protein or polypeptide (e.g., immunogen (or immunogenic fragment and/or immunogenic variant thereof)), wherein the amino acid sequence of the at least one SARS-CoV-2 spike protein or polypeptide (e.g., immunogen (or immunogenic fragment and/or immunogenic variant thereof)) comprises at least one set of amino acid substitutions set forth in Table 2; and (b) at least one SARS-CoV-2 spike protein or polypeptide (e.g., immunogen (or immunogenic fragment and/or immunogenic variant thereof)), wherein the amino acid sequence of the at least one SARS-CoV-2 spike protein or polypeptide (e.g., immunogen (or immunogenic fragment and/or immunogenic variant thereof)) comprises at least one set of amino acid substitutions set forth in Table 4. In some embodiments, the plurality comprises (a) at least 2, 3, 4, 5, or 6 SARS-CoV-2 spike proteins or polypeptides (e.g., immunogens (or immunogenic fragments and/or immunogenic variants thereof)), wherein the amino acid sequence of each of the 2, 3, 4, 5, or 6 SARS-CoV-2 spike proteins or polypeptides (e.g., immunogens (or immunogenic fragments and/or immunogenic variants thereof)) comprises a different set of amino acid substitutions set forth in Table 2 or a different combination of sets of amino acid substitutions set forth in Table 2; and (b) at least 2, 3, 4, 5, 6, 7, 8, 9, 10, 11, 12, 13, 14, 15, or 16 SARS-CoV-2 spike proteins or polypeptides (e.g., immunogens (or immunogenic fragments and/or immunogenic variants thereof)), wherein the amino acid sequence of each of said 2, 3, 4, 5, 6, 7, 8, 9, 10, 11, 12, 13, 14, 15, or 16 SARS-CoV-2 spike proteins or polypeptides (e.g., immunogens (or immunogenic fragments and/or immunogenic variants thereof)) comprises a different set of amino acid substitutions set forth in Table 4 or a different combination of sets of amino acid substitutions set forth in Table 4.

In some embodiments, the composition further comprises a (e.g., at least one) SARS-CoV-2 spike protein or polypeptide (e.g., immunogen (or immunogenic fragment and/or immunogenic variant thereof)), that does not comprise a set of amino acid substitutions set forth in Table 2 or Table 4. In some embodiments, the amino acid sequence of the SARS-CoV-2 spike protein is derived from a circulating strain of SARS-CoV-2. In some embodiments, the amino acid sequence of the SARS-CoV-2 spike protein comprises one or more amino acid variation found in one or more circulating variants of SARS-CoV-2. In some embodiments, the amino acid sequence of the SARS-CoV-2 spike protein is derived from a strain of SARS-CoV-2 known to have previously circulated. In some embodiments, the amino acid sequence of the SARS-CoV-2 spike protein comprises one or more amino acid variation found in one or more SARS-CoV-2 variants known to have previously circulated.

In some embodiments, the vaccine composition further comprises at least one immunogen (or immunogenic fragment and/or immunogenic variant thereof) from a non-SARS-CoV-2 virus, e.g., an influenza virus (e.g., influenza A, influenza B), a respiratory syncytial virus (RSV), a rhinovirus, a parvovirus, a parainfluenza virus, an adenovirus. In some embodiments, vaccine composition comprises one or more immunogen (or immunogenic fragment or immunogenic variant thereof) from an influenza virus (e.g., influenza A, influenza B), a respiratory syncytial virus (RSV), a rhinovirus, a parvovirus, a parainfluenza virus, and/or an adenovirus (or any combination thereof).

In some embodiments, the SARS-CoV-2 spike protein or polypeptide (e.g., a SARS-CoV-2 spike protein or polypeptide immunogen (or immunogenic fragment and/or immunogenic variant thereof)) (e.g., ectodomain of the SARS-CoV-2 spike protein or polypeptide) is modified to improve expression of the protein in host cells (e.g., insect cells, mammalian cells, eggs) as described below.

Polypeptides and proteins (e.g., immunogens) described herein, e.g., SARS-CoV-2 spike proteins and polypeptides (e.g., immunogens (and immunogenic fragments and/or immunogenic variants thereof)) may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the SARS-CoV-2 spike protein or polypeptide (e.g., the immunogen (or the immunogenic fragment and/or immunogenic variant thereof)). Such general methods are common knowledge in the art. The expression vector typically contains an expression cassette that includes nucleic acid sequences capable of bringing about expression of the nucleic acid molecule encoding the SARS-CoV-2 spike protein or polypeptide (e.g., the immunogen (or the immunogenic fragment and/or immunogenic variant thereof)), such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the SARS-CoV-2 spike protein or polypeptide (e.g., the immunogen (or the immunogenic fragment and/or immunogenic variant thereof)) are cultured under conditions conducive to expression of the nucleic acid molecule encoding the SARS-CoV-2 spike protein or polypeptide (e.g., the immunogen (or the immunogenic fragment and/or immunogenic variant thereof)). Culture media is available from various vendors, and a suitable medium can be routinely chosen for a host cell to express a polypeptide or protein of interest, here the SARS-CoV-2 spike protein or polypeptide (e.g., the immunogen (or the immunogenic fragment and/or immunogenic variant thereof)). Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced immunogenic peptide or protein may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The peptide or protein may be concentrated, buffer exchanged by ultrafiltration, and the retentate from the ultrafiltration may be filtered through an appropriate filter, e.g., a 0.22 μm filter. See, e.g., McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes.

The SARS-CoV-2 spike proteins and polypeptides (e.g., immunogens (or immunogenic fragments and/or immunogenic variants thereof)) described herein may also be produced synthetically. The SARS-CoV-2 spike proteins and polypeptides (e.g., immunogens (or immunogenic fragments and/or immunogenic variants thereof)) described herein may be produced by using an egg-based manufacturing method.

In some embodiments, the SARS-CoV-2 spike proteins and polypeptides (e.g., immunogens (or immunogenic fragments and/or immunogenic variants thereof)) of the vaccine composition are formulated in one or more carrier (e.g., a carrier described herein (see, e.g., § 5.7)).

In some embodiments, the vaccine compositions are pharmaceutical compositions (e.g., described herein, e.g., see § 5.8). In some embodiments, the vaccine compositions comprise an adjuvant (e.g., described herein, e.g., see § 5.9).

### 5.5.2 Nucleic Acid-Based Vaccines

In some embodiments, a nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.3) (or a fusion or conjugate thereof (see, e.g., § 5.4)) forms the basis for a vaccine composition (e.g., a prime vaccine composition, a booster composition, a vaccine prime and booster composition). Therefore, provided herein are, inter alia, vaccine compositions (e.g., prime vaccine compositions, booster compositions, vaccine prime and booster compositions) comprising a nucleic acid molecule comprising a coding region encoding at least one SARS-CoV-2 spike protein or polypeptide (e.g., a SARS-CoV-2 spike protein or polypeptide immunogen (or immunogenic fragment and/or immunogenic variant thereof)) described herein (see, e.g., § 5.3) (or a fusion or conjugate thereof (see, e.g., § 5.4)).

In some embodiments, the vaccine composition is a prime vaccine composition of a prime-boost vaccine regimen. In some embodiments, the vaccine composition is a vaccine booster composition of a prime-boost vaccine regimen. In some embodiments, the vaccine composition is a vaccine prime and booster composition of a prime-boost vaccine regimen. In some embodiments, the prime boost composition can be utilized as a prime and/or a booster (e.g., as described herein). In some embodiments, the vaccine composition forms a single dose vaccine that does not require a booster.

In some embodiments, the vaccine composition comprises a plurality of nucleic acid molecules, each comprising a coding region encoding at least one SARS-CoV-2 spike protein or polypeptide (e.g., a SARS-CoV-2 spike protein or polypeptide immunogen (or immunogenic fragment and/or immunogenic variant thereof)) (e.g., described herein).

In some embodiments, each of the nucleic acid molecules of the plurality are part of the same larger nucleic acid molecule. In some embodiments, each of the nucleic acid molecules of the plurality are separate (i.e., not connected) nucleic acid molecules. In some embodiments, at least two of the nucleic acid molecules of the plurality are part of the same larger nucleic acid molecule. In some embodiments, at least two of the nucleic acid molecules of the plurality are separate (i.e., not connected) nucleic acid molecules. In some embodiments, at least two of the nucleic acid molecules of the plurality are part of the same larger nucleic acid molecule; and at least one (e.g., at least 2, 3, 4, 5, etc.) of the nucleic acid molecules of the plurality is a separate (i.e., not connected) nucleic acid molecule.

In some embodiments, the plurality comprises at least 2, 3, 4, 5, 6, 7, 8, 9, 10, 11, 12, 13, 14, 15, 16, 17, 18, 19, 20, 30, 40, 50, 60, 70, 80, 90, 100 or more nucleic acid molecules. In some embodiments, the plurality comprises from about 2-100, 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleic acid molecules. In some embodiments, the plurality comprises at least 2 but no more than 100, 90, 80, 70, 60, 50, 40, 30, 20, 19, 18, 17, 16, 15, 14, 13, 12, 11, 10, 9, 8, 7, 6, 5, 4, 3 nucleic acid molecules.

In some embodiments, the amino acid sequence of each of the encoded SARS-CoV-2 spike proteins or polypeptides (e.g., immunogens (or immunogenic fragments and/or immunogenic variants thereof)) of the plurality is different.

In some embodiments, the amino acid sequence of at least one of the encoded SARS-CoV-2 spike proteins or polypeptides (e.g., immunogens (or immunogenic fragments and/or immunogenic variants thereof)) of the plurality is derived from a circulating strain of SARS-CoV-2.

In some embodiments, the plurality comprises at least one encoded SARS-CoV-2 spike protein or polypeptide (e.g., immunogen (or immunogenic fragment and/or immunogenic variant thereof)) described herein. In some embodiments, the plurality comprises at least one encoded SARS-CoV-2 spike protein or polypeptide (e.g., immunogen (or immunogenic fragment and/or immunogenic variant thereof)) described herein, wherein the amino acid sequence of the at least one encoded SARS-CoV-2 spike protein or polypeptide (e.g., immunogen (or immunogenic fragment and/or immunogenic variant thereof)) comprises a set of amino acid substitutions set forth in Table 2 or Table 4.

In some embodiments, the plurality comprises at least two encoded SARS-CoV-2 spike proteins or polypeptides (e.g., immunogens (or immunogenic fragments and/or immunogenic variants thereof)) described herein, wherein the amino acid sequence of each of the encoded at least two SARS-CoV-2 spike proteins or polypeptides (e.g., immunogens (or immunogenic fragments and/or immunogenic variants thereof)) comprises a different set of amino acid substitutions set forth in Table 2 or Table 4 or a different combination of sets of amino acid substitutions set forth in Table 2 or Table 4.

In some embodiments, the amino acid sequence of each of the encoded SARS-CoV-2 spike proteins or polypeptides (e.g., immunogens (or immunogenic fragments and/or immunogenic variants thereof)) of the plurality comprises a different set of amino acid substitutions described herein (e.g., in Table 2 or Table 4) or a different combination of sets of amino acid substitutions described herein (e.g., in Table 2 or Table 4).

In some embodiments, the plurality comprises (a) at least one encoded SARS-CoV-2 spike protein or polypeptide (e.g., immunogen (or immunogenic fragment and/or immunogenic variant thereof)), wherein the amino acid sequence of the at least one encoded SARS-CoV-2 spike protein or polypeptide (e.g., immunogen (or immunogenic fragment and/or immunogenic variant thereof)) comprises at least one set of amino acid substitutions set forth in Table 2; (b) at least one encoded SARS-CoV-2 spike protein or polypeptide (e.g., immunogen (or immunogenic fragment and/or immunogenic variant thereof)), wherein the amino acid sequence of the at least one encoded SARS-CoV-2 spike protein or polypeptide (e.g., immunogen (or immunogenic fragment and/or immunogenic variant thereof)) comprises at least one set of amino acid substitutions set forth in Table 4; and/or (c) at least one encoded SARS-CoV-2 spike protein or polypeptide (e.g., immunogen (or immunogenic fragment and/or immunogenic variant thereof)), wherein the amino acid sequence of the at least one encoded SARS-CoV-2 spike protein or polypeptide (e.g., immunogen (or immunogenic fragment and/or immunogenic variant thereof)) comprises at least one set of amino acid substitutions set forth in Table 2 and at least one set of amino acid substitutions set forth in Table 4.

In some embodiments, plurality comprises at least 2, 3, 4, 5, or 6 encoded SARS-CoV-2 spike proteins or polypeptides (e.g., immunogens (or immunogenic fragments and/or immunogenic variants thereof)), wherein the amino acid sequence of each of said 2, 3, 4, 5, or 6 encoded SARS-CoV-2 spike proteins or polypeptides (e.g., immunogens (or immunogenic fragments and/or immunogenic variants thereof)) comprises a different set of amino acid substitutions set forth in Table 2 or a different combination of sets of amino acid substitutions set forth in Table 2.

In some embodiments, the plurality comprises at least 2, 3, 4, 5, 6, 7, 8, 9, 10, 11, 12, 13, 14, 15, or 16 encoded SARS-CoV-2 spike proteins or polypeptides (e.g., immunogens (or immunogenic fragments and/or immunogenic variants thereof)), wherein the amino acid sequence of each of the 2, 3, 4, 5, 6, 7, 8, 9, 10, 11, 12, 13, 14, 15, or 16 encoded SARS-CoV-2 spike proteins or polypeptides (e.g., immunogens (or immunogenic fragments and/or immunogenic variants thereof)) comprises a different set of amino acid substitutions set forth in Table 4 or a different combination of sets of amino acid substitutions set forth in Table 4.

In some embodiments, plurality comprises (a) at least one encoded SARS-CoV-2 spike protein or polypeptide (e.g., immunogen (or immunogenic fragment and/or immunogenic variant thereof)), wherein the amino acid sequence of the at least one encoded SARS-CoV-2 spike protein or polypeptide (e.g., immunogen (or immunogenic fragment and/or immunogenic variant thereof)) comprises at least one set of amino acid substitutions set forth in Table 2; and (b) at least one encoded SARS-CoV-2 spike protein or polypeptide (e.g., immunogen (or immunogenic fragment and/or immunogenic variant thereof)), wherein the amino acid sequence of the at least one encoded SARS-CoV-2 spike protein or polypeptide (e.g., immunogen (or immunogenic fragment and/or immunogenic variant thereof)) comprises at least one set of amino acid substitutions set forth in Table 4. In some embodiments, the plurality comprises (a) at least 2, 3, 4, 5, or 6 encoded SARS-CoV-2 spike proteins or polypeptides (e.g., immunogens (or immunogenic fragments and/or immunogenic variants thereof)), wherein the amino acid sequence of each of the 2, 3, 4, 5, or 6 encoded SARS-CoV-2 spike proteins or polypeptides (e.g., immunogens (or immunogenic fragments and/or immunogenic variants thereof)) comprises a different set of amino acid substitutions set forth in Table 2 or a different combination of sets of amino acid substitutions set forth in Table 2; and (b) at least 2, 3, 4, 5, 6, 7, 8, 9, 10, 11, 12, 13, 14, 15, or 16 encoded SARS-CoV-2 spike proteins or polypeptides (e.g., immunogens (or immunogenic fragments and/or immunogenic variants thereof)), wherein the amino acid sequence of each of said 2, 3, 4, 5, 6, 7, 8, 9, 10, 11, 12, 13, 14, 15, or 16 encoded SARS-CoV-2 spike proteins or polypeptides (e.g., immunogens (or immunogenic fragments and/or immunogenic variants thereof)) comprises a different set of amino acid substitutions set forth in Table 4 or a different combination of sets of amino acid substitutions set forth in Table 4.

In some embodiments, the composition further comprises a nucleic acid molecule (e.g., at least one) comprising a coding region encoding a SARS-CoV-2 spike protein or polypeptide (e.g., immunogen (or immunogenic fragment and/or immunogenic variant thereof)), wherein the amino acid sequence of the encoded SARS-CoV-2 spike protein or polypeptide (e.g., immunogen (or immunogenic fragment and/or immunogenic variant thereof)) does not comprise a set of amino acid substitutions set forth in Table 2 or Table 4. In some embodiments, the amino acid sequence of the encoded SARS-CoV-2 spike protein is derived from a circulating strain of SARS-CoV-2. In some embodiments, the amino acid sequence of the encoded SARS-CoV-2 spike protein comprises one or more amino acid variation found in one or more circulating variants of SARS-CoV-2. In some embodiments, the amino acid sequence of the encoded SARS-CoV-2 spike protein is derived from a strain of SARS-CoV-2 known to have previously circulated. In some embodiments, the amino acid sequence of the encoded SARS-CoV-2 spike protein comprises one or more amino acid variation found in one or more SARS-CoV-2 variants known to have previously circulated.

In some embodiments, the vaccine composition further comprises at least one nucleic acid comprising a coding region encoding an immunogen (or immunogenic fragment and/or immunogenic variant thereof) from a non-SARS-CoV-2 virus, e.g., an influenza virus (e.g., influenza A, influenza B), a respiratory syncytial virus (RSV), a rhinovirus, a parvovirus, a parainfluenza virus, an adenovirus. In some embodiments, vaccine composition comprises one or more nucleic acid molecules encoding an immunogen (or immunogenic fragment or immunogenic variant thereof) from an influenza virus (e.g., influenza A, influenza B), a respiratory syncytial virus (RSV), a rhinovirus, a parvovirus, a parainfluenza virus, and/or an adenovirus (or any combination thereof).

In some embodiments, the encoded SARS-CoV-2 spike protein or polypeptide (e.g., a SARS-CoV-2 spike protein or polypeptide immunogen (or immunogenic fragment and/or immunogenic variant thereof)) (e.g., ectodomain of the SARS-CoV-2 spike protein or polypeptide) is modified to improve expression of the protein in host cells (e.g., insect cells, mammalian cells, eggs).

In some embodiments, the nucleic acid molecules are comprised within one or more vectors (e.g., vectors described herein (see, e.g., § 5.6)). In some embodiments, the nucleic acid molecules or the vectors of the vaccine composition are formulated in one or more carrier (e.g., a carrier described herein (see, e.g., § 5.7)).

In some embodiments, the vaccine compositions are pharmaceutical compositions (e.g., described herein, e.g., see § 5.8). In some embodiments, the vaccine compositions comprise an adjuvant (e.g., described herein, e.g., see § 5.9).

Nucleic acid molecules can be generated using common methods known in the art and described above in § 5.3.

### 5.6 Vectors

In some embodiments, the nucleic acid molecules described herein (e.g., DNA molecules, RNA molecules (e.g., mRNA molecules)) (see, e.g., § 5.3) (or a fusion or conjugate thereof (see, e.g., § 5.4)) are contained in a vector (e.g., a non-viral vector, a viral vector). Thus, also provided herein are, inter alia, vectors (e.g., viral vectors, non-viral vectors (e.g., plasmids, minicircles)) comprising one or more nucleic acid molecule described herein (e.g., nucleic acid molecules encoding a SARS-CoV-2 spike protein or polypeptide (e.g., a SARS-CoV-2 spike protein or polypeptide immunogen (or an immunogenic fragment and/or immunogenic variant thereof)). Such vectors can be easily manipulated by methods well known to the ordinary person of skill in the art.

In some embodiments, the vector is a non-viral vector (e.g., a plasmid, minicircle). In some embodiments, the vector is a plasmid. A person of ordinary skill in the art is aware of suitable plasmids for expression of the DNA of interest. For example, Suitable plasmid DNA may be generated to allow efficient production of the encoded peptides or proteins (e.g., SARS-CoV-2 proteins or polypeptides (e.g., immunogens) in cell lines, e.g., in insect cell lines, for example using vectors as described in WO2009150222A2 and as defined in PCT claims 1 to 33, the disclosure relating to claim 1 to 33 of WO2009150222A2 the entire contents of which is incorporated by reference herein for all purposes.

In some embodiments, the vector is a viral vector. Viral vectors include both RNA and DNA based vectors. The vectors can be designed to meet a variety of specifications. For example, viral vectors can be engineered to be capable or incapable of replication in prokaryotic and/or eukaryotic cells. In some embodiments, the vector is replication deficient. In some embodiments, the vector is replication competent. Viral vectors can be engineered or selected that either will (or will not) integrate in whole or in part into the genome of host cells, resulting (or not (e.g., episomal expression)) in stable host cells comprising the desired nucleic acid in their genome.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors. In some embodiments, the viral vector is an adenovirus vector, adeno-associated virus vector, or a lentivirus vector.

In some embodiments, the vector is an adenovirus vector (e.g., a human adenoviral vector, e.g., HAdV or AdHu). In some embodiments, the adenovirus vector has the E1 region deleted, rendering it replication-deficient in human cells. Other regions of the adenovirus such as E3 and E4 may also be deleted. Exemplary adenovirus vectors include, but are not limited to, those described in e.g., WO2005071093 or WQ2006048215, the entire contents of each of which is incorporated herein by reference for all purposes. In some embodiments, the adenovirus-based vector used is a simian adenovirus, thereby avoiding dampening of the immune response after vaccination by pre-existing antibodies to common human entities such as AdHu5. Exemplary, simian adenovirus vectors include AdCh63 (see, e.g., WO2005071093, the entire contents of which is incorporated herein by reference for all purposes) or AdCh68.

Viral vectors can be generated through the use of a packaging/producer cell line (e.g., a mammalian cell line) using standard methods known to the person of ordinary skill in the art. Generally, a nucleic acid construct (e.g., a plasmid) encoding the peptide or protein of interest (e.g., a peptide or protein described herein (e.g., SARS-CoV-2 peptide or protein described herein (e.g., a SARS-CoV-2 immunogen (or immunogenic fragment and/or immunogenic variant thereof)) (along with additional elements e.g., a promoter, inverted terminal repeats (ITRs) flanking the transgene, a plasmid encoding e.g., viral replication and structural proteins, along with one or more helper plasmids a host cell (e.g., a host cell line) are transfected into a host cell line (i.e., the packing/producer cell line). In some instances, depending on the viral vector, a helper plasmid may also be needed that include helper genes from another virus (e.g., in the instance of adeno-associated viral vectors). Eukaryotic expression plasmids are commercially available from a variety of suppliers, for example the plasmid series: pcDNA™, pCR3.1™, pCMV™, pFRT™, pVAX1™, pCI™, Nanoplasmid™ and Pcaggs. The person of ordinary skill in the art is aware of numerous transfection methods and any suitable method of transfection may be employed (e.g., using a biochemical substance as carrier (e.g., lipofectamine), by mechanical means, by electroporation). The cells are cultured under conditions suitable and for a sufficient time for plasmid expression. The viral particles may be purified from the cell culture medium using standard methods known to the person of ordinary skill in the art. For example, by centrifugation followed by e.g., chromatography and/or ultrafiltration.

### 5.7 Carriers

In some embodiments, a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), or a vector comprising a nucleic acid molecule comprising a coding region encoding the SARS-CoV-2 spike protein or polypeptide (e.g., a SARS-CoV-2 spike protein or polypeptide immunogen (or an immunogenic fragment and/or immunogenic variant thereof)) described herein is formulated within one or more carrier.

Therefore, further provided herein are carriers comprising any one or more of a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein).

Any of the foregoing (e.g., one or more of a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein)) can be encapsulated within a carrier, chemically conjugated to a carrier, associated with the carrier. In this context, the term “associated” refers to the essentially stable combination of an agent described herein (or a conjugate comprising the same) (or a vector comprising the same) with one or more molecules of a carrier (e.g., one or more lipids of a lipid-based carrier, e.g., an LNP, liposome, lipoplex, and/or nanoliposome) into larger complexes or assemblies without covalent binding. In this context, the term “encapsulation” refers to the incorporation of a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) into a carrier (e.g., a lipid-based carrier, e.g., an LNP, liposome, lipoplex, and/or nanoliposome) wherein the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) is entirely contained within the interior space of the carrier (e.g., the lipid-based carrier, e.g., the LNP, liposome, lipoplex, and/or nanoliposome).

Exemplary carriers includes, but are not limited to, lipid-based carriers (e.g., lipid nanoparticles (LNPs), liposomes, lipoplexes, and nanoliposomes). In some embodiments, the carrier is a lipid-based carrier. In some embodiments, the carrier is an LNP. In some embodiments, the LNP comprises a cationic lipid, a neutral lipid, a cholesterol, and/or a PEG lipid. Lipid based carriers are further described below in § 5.7.1.

### 5.7.1 Lipid Based Carriers/Lipid Nanoformulations

In some embodiments, a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) is encapsulated or associated with one or more lipids (e.g., cationic lipids and/or neutral lipids), thereby forming lipid-based carriers such as lipid nanoparticles (LNPs), liposomes, lipoplexes, or nanoliposomes.

In some embodiments, a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) is encapsulated in one or more lipids (e.g., cationic lipids and/or neutral lipids), thereby forming lipid-based carriers such as lipid nanoparticles (LNPs), liposomes, lipoplexes, or nanoliposomes. In some embodiments, a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) is associated with one or more lipids (e.g., cationic lipids and/or neutral lipids), thereby forming lipid-based carriers such as lipid nanoparticles (LNPs), liposomes, lipoplexes, or nanoliposomes. In some embodiments, a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) is encapsulated in LNPs (e.g., as described herein).

The SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) may be completely or partially located in the interior space of the LNPs, liposomes, lipoplexes, and/or nanoliposomes, within the lipid layer/membrane, or associated with the exterior surface of the lipid layer/membrane. One purpose of incorporating a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) into LNPs, liposomes, lipoplexes, and/or nanoliposomes is to protect the agent from an environment which may contain enzymes or chemicals or conditions that degrade the agent from molecules or conditions that cause the rapid excretion of the agent. Moreover, incorporating a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) into LNPs, liposomes, lipoplexes, and/or nanoliposomes may promote the uptake of the agent, and hence, may enhance the therapeutic effect of the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein). Accordingly, incorporating a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein), into LNPs, liposomes, lipoplexes, and/or nanoliposomes may be particularly suitable for a pharmaceutical composition described herein, e.g., for intramuscular and/or intradermal administration.

In some embodiments, a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) is formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), nucleic acid molecule comprising a coding region encoding the foregoing (e.g., a nucleic acid molecule described herein), or vector comprising the foregoing nucleic acid molecule (e.g., a vector described herein), or a pharmaceutically acceptable salt thereof.

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), nucleic acid molecule comprising a coding region encoding the foregoing (e.g., a nucleic acid molecule described herein), or vector comprising the foregoing nucleic acid molecule (e.g., a vector described herein), the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

### 5.7.1.1 Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in e.g. Martin et al., Current Pharmaceutical Design, pages 1-394, the entire contents of which are incorporated by reference herein for all purposes. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, the entire contents of which are incorporated by reference herein for all purposes.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

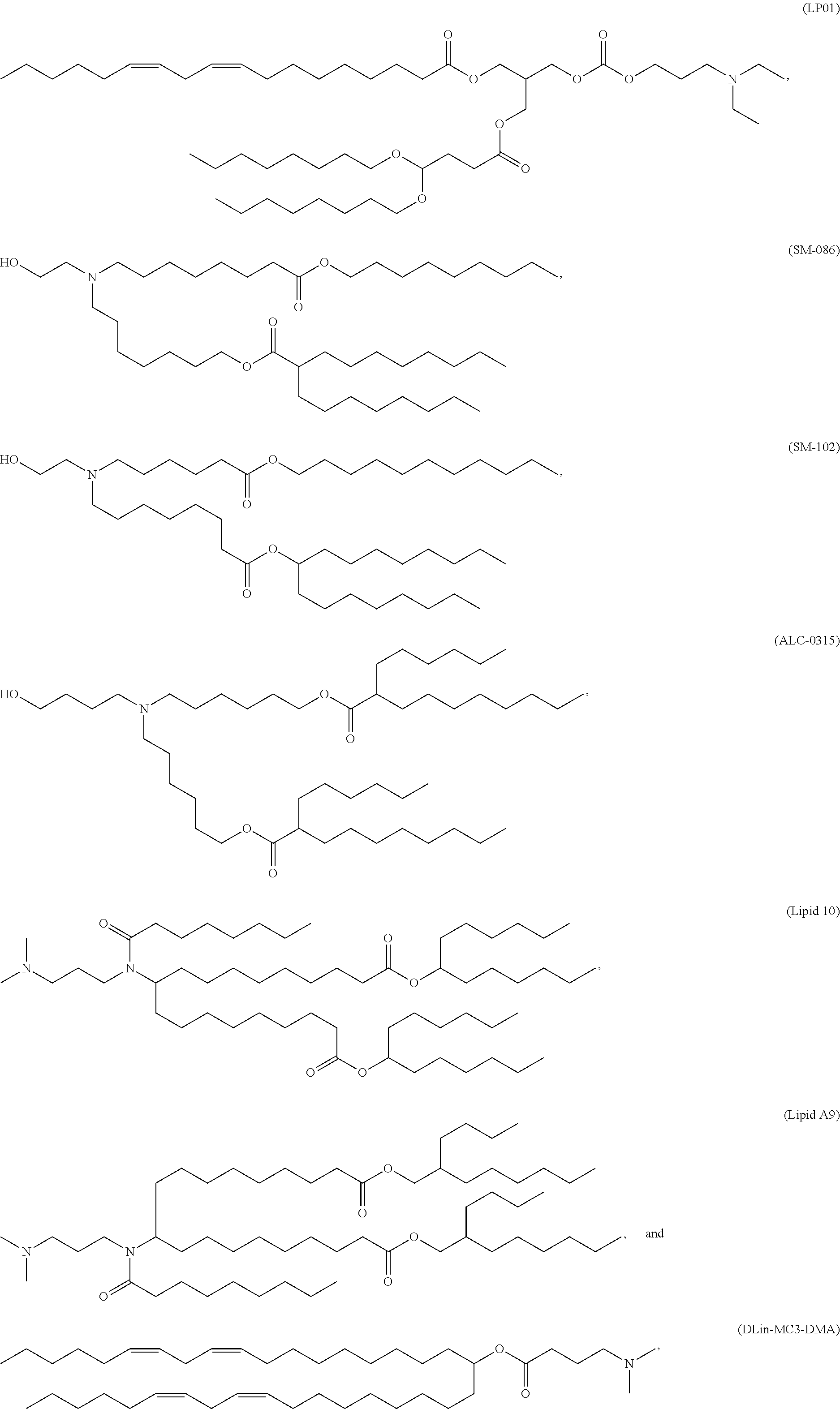

(see WO2017004143A1, the entire contents of which is incorporated herein by reference for all purposes).

In some embodiments, the lipid-based carrier (or lipid nanoformulation) further comprises one or more compounds described by WO 2021/113777 (e.g., a lipid of Formula (3) such as a lipid of Table 3 of WO 2021/113777), the entire contents of which are incorporated by reference herein for all purposes.

In one embodiment, the ionizable lipid is a lipid disclosed in Hou, X., et al. Nat Rev Mater 6, 1078-1094 (2021). https://doi.org/10.1038/s41578-021-00358-0 (e.g., L319, C12-200, and DLin-MC3-DMA), (the entire contents of which are incorporated by reference herein for all purposes).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y-Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; III-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U.S. Pat. No. 9,708,628; I of WO 2020/106946; I of WO 2020/106946; (1), (2), (3), or (4) of WO 2021/113777; and any one of Tables 1-16 of WO 2021/113777, the entire contents of each of which are incorporated by reference herein for all purposes.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, the entire contents of each of which are incorporated by reference herein for all purposes.

### 5.7.1.2 Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, the entire contents of which are incorporated by reference herein for all purposes. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, the entire contents of each of which are incorporated by reference herein for all purposes.

In one embodiment, the lipid-based carrier (or lipid nanoformulation) further comprises one or more non-cationic lipid that is oleic acid or a compound of Formula I, II, or IV of US 2018/0028664, the entire contents of which are incorporated by reference herein for all purposes.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

### 5.7.1.3 Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, the entire contents of each of which are incorporated by reference herein for all purposes. Additional exemplary sterols include phytosterols, including those described in Eygeris et al. (2020), Nano Lett. 2020; 20(6):4543-4549, the entire contents of which are incorporated by reference herein for all purposes.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, the entire contents of each of which are incorporated by reference herein for all purposes.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### 5.7.1.4 Polymers and Polyethylene Glycol (PEG)-Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-350] (mPEG 350 PE); 1,2-Diacyl-sn-Glycero-3-Phosphoethanolamine-N-[Methoxy(Polyethylene glycol)-550] (mPEG 550 PE); 1,2-Diacyl-sn-Glycero-3-Phosphoethanolamine-N-[Methoxy(Polyethylene glycol)-750] (mPEG 750 PE); 1,2-Diacyl-sn-Glycero-3-Phosphoethanolamine-N-[Methoxy(Polyethylene glycol)-1000](mPEG 1000 PE); 1,2-Diacyl-sn-Glycero-3-Phosphoethanolamine-N-[Methoxy(Polyethylene glycol)-2000] (mPEG 2000 PE); 1,2-Diacyl-sn-Glycero-3-Phosphoethanolamine-N-[Methoxy(Polyethylene glycol)-3000] (mPEG 3000 PE); 1,2-Diacyl-sn-Glycero-3-Phosphoethanolamine-N-[Methoxy(Polyethylene glycol)-5000] (mPEG 5000 PE); N-Acyl-Sphingosine-1-[Succinyl(Methoxy Polyethylene Glycol) 750] (mPEG 750 Ceramide); N-Acyl-Sphingosine-1-[Succinyl(Methoxy Polyethylene Glycol) 2000] (mPEG 2000 Ceramide); and N-Acyl-Sphingosine-1-[Succinyl(Methoxy Polyethylene Glycol) 5000] (mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, the entire contents of which are incorporated by reference herein for all purposes). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (the entire contents of which are incorporated by reference herein for all purposes), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, the entire contents of each of which are incorporated by reference herein for all purposes.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, the entire contents of each of which are incorporated by reference herein for all purposes. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

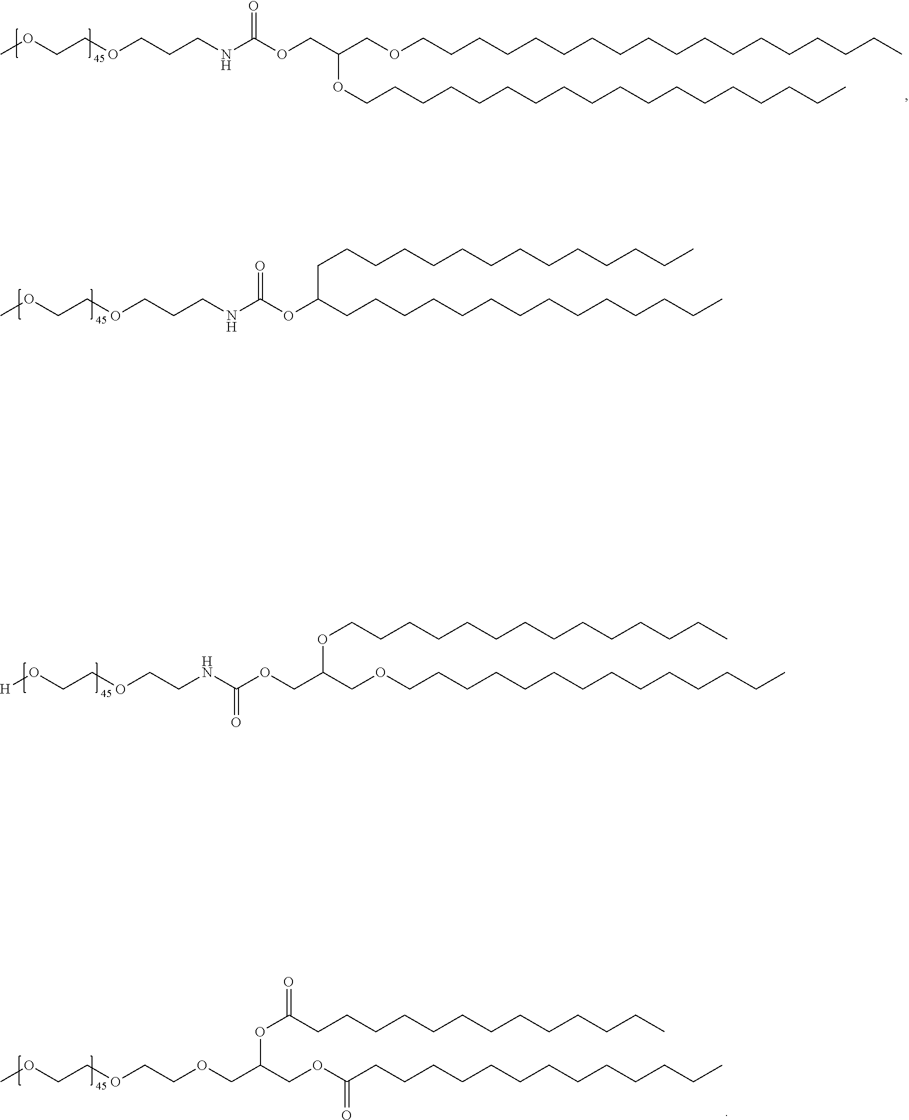

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, the entire contents of which are incorporated by reference herein for all purposes.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), the entire contents of each of which are incorporated by reference herein for all purposes.

### 5.7.1.5 Percentages of Lipid Nanoformulation Components

In some embodiments, the lipid-based carrier (or lipid nanoformulation) comprises one of more of a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein), optionally a non-cationic lipid (e.g., a phospholipid), a sterol, a neutral lipid, and optionally conjugated lipid (e.g., a PEGylated lipid) that inhibits aggregation of particles. In some embodiments, the lipid-based carrier (or lipid nanoformulation) further comprises a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein). The amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 100%.

In some embodiments, the lipid-based carrier (or lipid nanoformulation) comprises about 25-100 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) that is formulated in a lipid nanoparticle, wherein the lipid nanoparticle comprises about 25-100 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) may be at least 70%.

In one embodiment, the lipid-based carrier (or lipid nanoformulation) comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) that is formulated in a lipid nanoparticle, wherein the lipid nanoparticle comprises about 25-100 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) that is formulated in a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) may be at least 70%.

In some embodiments, molar ratios of ionizable lipid/sterol/phospholipid (or another structural lipid)/PEG-lipid/additional components is varied in the following ranges: ionizable lipid (25-100%); phospholipid (DSPC) (0-40%); sterol (0-50%); and PEG lipid (0-5%).

In some embodiments, the lipid-based carrier comprises a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) that is formulated in a lipid nanoparticle, wherein the lipid nanoparticle comprises molar ratios of ionizable lipid/sterol/phospholipid (or another structural lipid)/PEG-lipid/additional components in the following ranges: ionizable lipid (25-100%); phospholipid (DSPC) (0-40%); sterol (0-50%); and PEG lipid (0-5%). In some embodiments, the encapsulation efficiency of the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

In some embodiments, the lipid-based carrier comprises a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein) that is formulated in a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) may be at least 70%.

In some embodiments, the lipid-based carrier (or lipid nanoformulation) comprises (i) a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein); (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein); (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (d) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) can be varied as desired. For example, the total lipid components to the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) (mass or weight) ratio can be from about 10:1 to about 30:1. In some embodiments, the total lipid components to the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation's) overall lipid content can range from about 5 mg/ml to about 30 mg/mL. Nitrogen:phosphate ratios (N:P ratio) is evaluated at values between 0.1 and 100.

The efficiency of encapsulation of a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the foregoing (e.g., a nucleic acid molecule described herein), or a vector comprising the foregoing nucleic acid molecule (e.g., a vector described herein), describes the amount of the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) that is encapsulated or otherwise associated with a lipid nanoformulation (e.g., liposome or LNP) after preparation, relative to the initial amount provided. The encapsulation efficiency is desirably high (e.g., at least 70%, 80%. 90%, 95%, close to 100%). The encapsulation efficiency may be measured, for example, by comparing the amount of the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) in a solution containing the liposome or LNP before and after breaking up the liposome or LNP with one or more organic solvents or detergents. An anion exchange resin may be used to measure the amount of free the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) in a solution. Fluorescence may be used to measure the amount of free the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), the nucleic acid molecule comprising a coding region encoding the foregoing (e.g., a nucleic acid molecule described herein), or the vector comprising the foregoing nucleic acid molecule (e.g., a vector described herein) in a solution. For the lipid-based carrier (or lipid nanoformulation) described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

### 5.8 Pharmaceutical Compositions

Provided herein are, inter alia, pharmaceutical compositions comprising a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule described herein (e.g., a nucleic acid molecule comprising a coding region encoding a SARS-CoV-2 spike protein or polypeptide (e.g., a SARS-CoV-2 spike protein or polypeptide immunogen (or an immunogenic fragment and/or immunogenic variant thereof)) (or a fusion or conjugate thereof), a vector described herein (e.g., a vector comprising a nucleic acid molecule described herein (e.g., a nucleic acid molecule comprising a coding region encoding a SARS-CoV-2 spike protein or polypeptide (e.g., a SARS-CoV-2 spike protein or polypeptide immunogen (or an immunogenic fragment and/or immunogenic variant thereof)), a carrier described herein (e.g., a carrier comprising a SARS-CoV-2 spike protein or polypeptide (e.g., a SARS-CoV-2 spike protein or polypeptide immunogen (or an immunogenic fragment and/or immunogenic variant thereof)) described herein or a nucleic acid molecule described herein (e.g., a nucleic acid molecule comprising a coding region encoding a SARS-CoV-2 spike protein (e.g., a SARS-CoV-2 spike protein or polypeptide immunogen (or an immunogenic fragment and/or immunogenic variant thereof))), and/or a composition described herein (e.g., a vaccine composition), and a pharmaceutically acceptable excipient (see, e.g., Remington's Pharmaceutical Sciences (1990) Mack Publishing Co., Easton, PA, the entire contents of which is incorporated by reference herein for all purposes).

In one aspect, also provided herein are methods of making pharmaceutical compositions described herein comprising providing a SARS-CoV-2 spike protein or polypeptide (e.g., a SARS-CoV-2 spike protein or polypeptide immunogen (or an immunogenic fragment and/or immunogenic variant thereof)) described herein, a nucleic acid molecule described herein (e.g., a nucleic acid molecule comprising a coding region encoding a SARS-CoV-2 spike protein (e.g., a SARS-CoV-2 spike protein or polypeptide immunogen (or an immunogenic fragment and/or immunogenic variant thereof)) described herein), a vector described herein (e.g., a vector comprising a nucleic acid molecule described herein (e.g., a nucleic acid molecule comprising a coding region encoding a SARS-CoV-2 spike protein or polypeptide (e.g., a SARS-CoV-2 spike protein or polypeptide immunogen (or an immunogenic fragment and/or immunogenic variant thereof)))), a carrier described herein (e.g., a carrier comprising a SARS-CoV-2 spike protein (or polypeptide e.g., immunogen (or an immunogenic fragment and/or immunogenic variant thereof)) described herein or a nucleic acid molecule described herein (e.g., a nucleic acid molecule encoding a SARS-CoV-2 spike protein or polypeptide (e.g., a SARS-CoV-2 spike protein or polypeptide immunogen (or an immunogenic fragment and/or immunogenic variant thereof))), and/or a composition described herein (e.g., a vaccine composition), and formulating it into a pharmaceutically acceptable composition by the addition of one or more pharmaceutically acceptable excipient.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

A pharmaceutical composition may be formulated for any route of administration to a subject. The skilled person knows the various possibilities to administer a pharmaceutical composition described herein a in order to induce an immune response to the immunogens(s) and/or antigen(s) in the pharmaceutical composition. Non-limiting embodiments include parenteral administration, such as intramuscular, intradermal, subcutaneous, transcutaneous, or mucosal administration, e.g., inhalation, intranasal, oral, and the like. In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; orsodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.9 Adjuvants

Any of the foregoing, e.g., SARS-CoV-2 spike proteins or polypeptides (e.g., immunogens (or immunogenic fragments and/or immunogenic variants thereof)) described herein (or a fusion or conjugate thereof), nucleic acid molecules described herein (e.g., nucleic acid molecules comprising a coding region encoding a SARS-CoV-2 spike protein or polypeptide (e.g., a SARS-CoV-2 spike protein or polypeptide immunogen (or an immunogenic fragment and/or immunogenic variant thereof)) (or a fusion or conjugate thereof), vectors described herein (e.g., vectors comprising a nucleic acid molecule described herein (e.g., a nucleic acid molecule comprising a coding region encoding a SARS-CoV-2 spike protein or polypeptide (e.g., a SARS-CoV-2 spike protein or polypeptide immunogen (or an immunogenic fragment and/or immunogenic variant thereof)), carriers described herein (e.g., a carrier comprising a SARS-CoV-2 spike protein or polypeptide (e.g., a SARS-CoV-2 spike protein or polypeptide immunogen (or an immunogenic fragment and/or immunogenic variant thereof)) described herein or a nucleic acid molecule described herein (e.g., a nucleic acid molecule comprising a coding region encoding a SARS-CoV-2 spike protein or polypeptide (e.g., a SARS-CoV-2 spike protein or polypeptide immunogen (or an immunogenic fragment and/or immunogenic variant thereof))), compositions described herein (e.g., a vaccine composition), and/or pharmaceutical compositions described herein may be co-formulated with and/or administered in combination with an adjuvant.

Adjuvants are known in the art to further increase the immune response (e.g., to an immunogen). General categories of adjuvants include, but are not limited to, inorganic adjuvants, small molecule adjuvants, oil in water emulsions, lipids, polymers, peptides, peptidoglycans, carbohydrates, polysaccharides, RNA-based adjuvants, DNA-based adjuvants, viral particles, bacterial adjuvants, nanoparticles (e.g., inorganic nanoparticles), and multi-component adjuvants. Examples of adjuvants include, but are not limited to, aluminum salts such as aluminum hydroxide and/or aluminum phosphate; oil-emulsion compositions (or oil-in-water compositions), including squalene-water emulsions, such as MF59 (see, e.g., WO90/14837, the entire contents of which is incorporated herein by reference for all purposes), MF59, AS03, and Montanide; saponin formulations, such as for example QS21 and Immunostimulating Complexes (ISCOMS) (see, e.g., U.S. Pat. No. 5,057,540; WO90/03184, WO96/11711, WO2004/004762, WO2005/002620, the entire contents of each of which is incorporated herein by reference for all purposes); protamine or a protamine salt (e.g., protamine sulfate); calcium salt; bacterial or microbial derivatives, examples of which include monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g., antibodies or fragments thereof (e.g., directed against the antigen itself or CD1a, CD3, CD7, CD80) and ligands to receptors (e.g., CD40L, GMCSF, GCSF, etc.).

Exemplary RNA-based adjuvants include, but are not limited to, Poly IC, Poly IC:LC, hairpin RNAs, e.g., with a 5′PPP containing sequence, viral sequences, polyU containing sequences, dsRNA, natural or synthetic immunostimulatory RNA sequences, nucleic acids analogs, optionally cyclic GMP-AMP or a cyclic dinucleotide such as cyclic di-GMP, and immunostimulatory base analogs, e.g., C8-substitued or an N7,C8-disubstituted guanine ribonucleotide. Exemplary DNA-based adjuvants, include, but are not limited to, CpGs, dsDNA, or natural or synthetic immunostimulatory DNA sequences. Exemplary bacteria-based adjuvants include, but are not limited, to bacterial adjuvant is flagellin, LPS, or a bacterial toxin, e.g., enterotoxins, heat-labile toxins, and Cholera toxins. Exemplary carbohydrate or polysaccharide adjuvants include, but are not limited to, dextran (branched microbial polysaccharide), dextran-sulfate, Lentinan, zymosan, Betaglucan, Deltin, Mannan, and Chitin. Exemplary small molecule adjuvants, include, but are not limited to, imiquimod, resiquimod, and gardiquimod. Exemplary lipid or polymer adjuvants, include, but are not limited to, polymeric nanoparticles (e.g., PLGA, PLG, PLA, PGA, or PHB), liposomes (e.g., Virosomes and CAFO1), LNPs or a component thereof, lipopolysaccharide (LPS) (e.g., monophosphoryl lipid A (MPLA) or glucopyranosyl Lipid A (GLA)), lipopeptides (e.g., Pam2 (Pam2CSK4) or Pam3 (Pam3CSK4)), and glycolipid (e.g., trehalose dimycolate). Exemplary peptides or peptidoglycan include, but are not limited to, N-acetyl-muramyl-L-alanyl-D-isoglutamine (MDP), flagellin-fusion protein, mannose-binding lectin (MBL), cytokines, and chemokine. Exemplary inorganic nanoparticle adjuvants, include, but are not limited to, gold nanorods, silica-based nanoparticles (e.g., mesoporous silica nanoparticles (MSN)). Exemplary multicomponent adjuvants include, but are not limited to, ASO1, ASO3, ASO4, Complete Freunds Adjuvant, and CAFO1.

### 5.10 Host Cells

Provided herein are host cells comprising a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a vector comprising a nucleic acid molecule comprising a coding region encoding the SARS-CoV-2 spike protein or polypeptide (e.g., a SARS-CoV-2 spike protein or polypeptide immunogen (or an immunogenic fragment and/or immunogenic variant thereof)); a carrier described herein, a composition described herein, a vaccine composition described herein, or a pharmaceutical composition described herein. In some embodiments, the host cell (or population of host cells) is in vitro. In some embodiments, the host cell (or population of host cells) is in vivo. In some embodiments, the host cell (or population of host cells) is ex vivo.

### 5.11 Methods of Making Polypeptides & Proteins

Proteins described herein (e.g., SARS-CoV-2 spike proteins or polypeptides (e.g., SARS-CoV-2 spike protein or polypeptide immunogens (or immunogenic fragments and/or immunogenic variants thereof)), fusion proteins, and protein conjugates, fusion proteins, and protein conjugates) may be produced using standard methods known in the art. For example, each may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the protein of interest (e.g., the SARS-CoV-2 spike protein or polypeptide (e.g., the SARS-CoV-2 spike protein or polypeptide immunogen (or immunogenic fragment and/or immunogenic variant thereof)). Such general methods are common knowledge in the art. The expression vector typically contains an expression cassette that includes nucleic acid sequences capable of bringing about expression of the nucleic acid molecule encoding the protein of interest, such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein of interest are cultured under conditions conducive to expression of the nucleic acid molecule encoding the protein. Culture media is available from various vendors, and a suitable medium can be routinely chosen for a host cell to express a protein of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein may be concentrated, buffer exchanged by ultrafiltration, and the retentate from the ultrafiltration may be filtered through an appropriate filter, e.g., a 0.22 μm filter. See, e.g., Hacker, David (Ed.), Recombinant Protein Expression in Mammalian Cells: Methods and Protocols (Methods in Molecular Biology), Humana Press (2018); and McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes.

Proteins described herein (e.g., SARS-CoV-2 spike proteins or polypeptides (e.g., SARS-CoV-2 spike protein or polypeptide immunogens (or immunogenic fragments and/or immunogenic variants thereof)), fusion proteins, and protein conjugates) may be produced synthetically. Proteins described herein, and particularly the immunogenic proteins described herein, may be produced by using an egg-based manufacturing method.

In embodiments, the invention features methods of making the proteins described herein (e.g., the SARS-CoV-2 spike proteins or polypeptides (e.g., SARS-CoV-2 spike protein or polypeptide immunogens (or immunogenic fragments and/or immunogenic variants thereof)), fusion proteins, and protein conjugates). In some embodiments, the method comprises introducing into a cell or a population of population of cells a nucleic acid molecule (or a vector or carrier comparing the same) into the host cell or population of host cells; culturing the cell or population of cells in a culture medium under conditions suitable for expression of the protein; isolating the protein from the culture medium; and optionally purifying the protein. In some embodiments the cell (or population of cells) is in vitro. In some embodiments the cell (or population of cells) is in in vivo. In some embodiments the cell (or population of cells) is ex vivo.

In some embodiments, the method comprises (a) recombinantly expressing a protein described herein (e.g., a SARS-CoV-2 spike protein or polypeptide (e.g., SARS-CoV-2 spike protein or polypeptide immunogen (or immunogenic fragment and/or immunogenic variant thereof)), fusion protein, or protein conjugate); (b) enriching, e.g., purifying, the protein; (c) evaluating the protein described herein for the presence of a process impurity or contaminant, and (d) formulating the protein as a pharmaceutical composition if the protein meets a threshold specification for the process impurity or contaminant. The process impurity or contaminant evaluated may be one or more of, e.g., a process-related impurity such as host cell proteins, host cell DNA, or a cell culture component (e.g., inducers, antibiotics, or media components); a product-related impurity (e.g., precursors, fragments, aggregates, degradation products); or contaminants, e.g., endotoxin, bacteria, viral contaminants.

### 5.12 Methods of Making Nucleic Acid Molecules

Nucleic acid molecules described herein can be generated using common methods known in the art (e.g., chemical synthesis).

In some embodiments, the nucleic acid molecule is modified or varied (compared to the sequence of a reference nucleic acid molecule), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide variation (see, e.g., description below), etc.

In some embodiments, the sequence of the nucleic acid molecule is codon optimized, e.g., for expression in humans. Codon optimization, in some embodiments, may be used to match codon frequencies in target and host organisms to ensure proper folding; bias guanosine (G) and/or cytosine (C) content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g. glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified or varied to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 5.13 Methods of Use

Provided herein are various methods of utilizing the SARS-CoV-2 spike proteins and polypeptides (e.g., immunogens (and immunogenic fragments and/or immunogenic variants thereof)) described herein (or fusions or conjugates thereof), the nucleic acid molecules described herein (e.g., nucleic acid molecules comprising a coding region encoding a SARS-CoV-2 spike protein or polypeptide (e.g., a SARS-CoV-2 spike protein or polypeptide immunogen (or immunogenic fragment and/or immunogenic variant thereof))) (or fusions or conjugates thereof), the vectors described herein (e.g., vectors comprising a nucleic acid molecule described herein (e.g., a nucleic acid molecule comprising a coding region encoding a SARS-CoV-2 spike protein e.g., immunogen (or immunogenic fragment and/or immunogenic variant thereof)), the carriers described herein (e.g., carriers comprising a SARS-CoV-2 spike protein or polypeptide (e.g., a SARS-CoV-2 spike protein or polypeptide immunogen (or immunogenic fragment and/or immunogenic variant thereof) or a nucleic acid molecule described herein (e.g., a nucleic acid molecule comprising a encoding a SARS-CoV-2 spike protein or polypeptide (e.g., a SARS-CoV-2 spike protein or polypeptide immunogen (or immunogenic fragment and/or immunogenic variant thereof), vaccine compositions (e.g., a vaccine composition comprising any of the foregoing), and/or pharmaceutical compositions described herein (e.g., a pharmaceutical composition comprising any of the foregoing).

Some of the following methods include administering one or more of the foregoing (e.g., protein (or a fusion or conjugate thereof), polypeptide (or a fusion or conjugate thereof), immunogen (or a fusion or conjugate thereof), nucleic acid molecule (or a fusion or conjugate thereof), vector, carrier, vaccine composition, pharmaceutical composition) to a subject. Exemplary subjects include mammals, e.g., humans, non-human mammals, e.g., non-human primates. In some embodiments, the subject is a human.

In some embodiments, the subject is elderly, pregnant, a newborn, immunocompromised, or immunosuppressed. In some embodiments, the subject has one or more of the following cancer, heart disease, obesity, diabetes, asthma, chronic lung disease, and/or sickle cell disease. In some embodiments, the subject has a weakened immune system or weakened immune response (e.g., a weakened immune response to a vaccine). In some embodiments, the subject is immunocompromised or immunosuppressed. In some embodiments, the subject is clinically vulnerable to the infection. In some embodiments, the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, and/or is at least 50 (e.g., at least 55, 60, 65, 70, 75, 80, 85, 90, or 100) years of age. In some embodiments, the subject at least 50, 60, 65, 70, or 75 years of age. In some embodiments, the subject is at least 50, 55, 60, 65, 70, 75, 80, 85, 90, 100, 110, or 120 years of age. In some embodiments, the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

The dosage of one or more of the foregoing (e.g., protein, polypeptide, immunogen, nucleic acid molecule, vector, carrier, vaccine composition, pharmaceutical composition) to be administered to a subject can be determined in accordance with standard techniques well known to those of ordinary skill in the art, including the type (if any) adjuvant is used, the route of administration, and the age and weight of the subject. In some embodiments, a single dose of any one of the foregoing is administered to a subject in need thereof. In some embodiments, a series of doses of any one of the foregoing are administered to a subject in need thereof (e.g., two doses given at a set interval (e.g., 2 weeks, 3 weeks) apart or within a range (e.g., 2-6 weeks apart)). In some embodiments, the SARS-CoV-2 spike protein or polypeptide (e.g., a SARS-CoV-2 spike protein or polypeptide immunogen (or immunogenic fragment and/or immunogenic variant thereof)) described herein, the nucleic acid molecule described herein, the vector described herein, the carrier described herein, the vaccine composition, or the pharmaceutical composition described herein (e.g., any one of the foregoing) is administered in a therapeutically effective amount. In some embodiments, a dose of an mRNA molecule encoding a SARS-CoV-2 spike protein or polypeptide (e.g., a SARS-CoV-2 spike protein or polypeptide immunogen (or immunogenic fragment and/or immunogenic variant thereof)) described herein (e.g., a vaccine or pharmaceutical composition comprising the same) is between 30-200 mcg, e.g., 30 mcg, 50 mcg, 75 mcg, 100 mcg, 150 mcg, or 200 mcg.

### 5.13.1 Methods of Delivery

Provided herein are methods of delivering (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein or polypeptide described herein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule) comprising a coding region encoding a SARS-CoV-2 spike protein or polypeptide described herein (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing) to a subject in need thereof, the method comprising administering to the subject (a) the SARS-CoV-2 spike protein or polypeptide (e.g., the SARS-CoV-2 spike protein or polypeptide immunogen (or immunogenic fragment and/or immunogenic variant thereof)), (b) the nucleic acid molecule (e.g., the RNA molecule, e.g., the mRNA molecule), (c) the vector, (d) the carrier, (e) the vaccine composition, or (f) the pharmaceutical composition to the subject, to thereby deliver the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition to the subject.

In some embodiments, the SARS-CoV-2 spike protein or polypeptide (e.g., the SARS-CoV-2 spike protein or polypeptide immunogen (or immunogenic fragment and/or immunogenic variant thereof)), the nucleic acid molecule, the vector, the carrier, the vaccine composition, or the pharmaceutical composition is administered to the subject in an amount and for a time sufficient to deliver the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., an mRNA molecule), the vector, the carrier, the vaccine composition, or the pharmaceutical composition to the subject.

### 5.13.2 Methods of Inducing or Enhancing an Immune Response

Provided herein are methods of inducing and/or enhancing an immune response in a subject in need thereof, the method comprising administering to the subject (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing), to thereby induce and/or enhance an immune response in the subject.

In some embodiments, the method comprises a method of inducing an immune response in the subject in need thereof. In some embodiments, the method comprises a method of enhancing an immune response in the subject in need thereof. In some embodiments, the method comprises a method of including and enhancing an immune response in the subject in need thereof.

In some embodiments, the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to induce and/or enhance an immune response the subject. In some embodiments, the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to induce an immune response the subject. In some embodiments, the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to induce enhance an immune response the subject. In some embodiments, the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to induce and enhance an immune response the subject.

Further provided herein are (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing) for use in the manufacture of a medicament for the induction and/or enhancement of an immune response in a subject in need thereof.

Further provided herein are (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing) for use in a method of inducing and/or enhancing an immune response in a subject need in thereof, the method comprising administering the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition to the subject in need thereof.

An immune response in a subject can be measured by common methods known to those of skill in the art. For example, serological assays can be employed to detect a humoral response by measuring titers of anti-antigen (e.g., anti-SARS-CoV-2 spike protein, anti-SARS-CoV-2 spike protein RBD) IgG antibodies post administration. For example, an enzyme-linked immunosorbent assay (ELISA) is a standard laboratory test for detecting and quantifying antibodies well known to the person of skill in the art. Generally, blood is collected from a consenting subject, centrifuged, and the serum isolated according to standard techniques. The recombinant target antigen (e.g., SARS-CoV-2 spike protein, SARS-CoV-2 spike protein RBD) is immobilized in microplate wells. The microplate is blocked by through the incubation with an irrelevant antigen (e.g., bovine serum albumin). The serum sample from the subject is prepared and added to the blocked wells to allow for binding of an antigen specific antibodies to the immobilized antigen. The bound antibodies are detected using a secondary tagged antibody that binds to the previously bound antibodies (e.g., anti-human IgG antibodies). See, e.g., Front. Immunol., Forgacs David et al., SARS-CoV-2 mRNA Vaccines Elicit Different Responses in Immunologically Naïve and Pre-Immune Humans; Vol 12 (27 Sep. 2021) https://doi.org/10.3389/fimmu.2021.728021, the entire contents of which is incorporated by reference herein for all purposes.

Cell based assays can also be utilized to detect a cell based immune response (e.g., T cell immune response). For example, antigen specific T cells (e.g., CD4+ or CD8+ T cells) can be measured using an enzyme-linked immunospot (ELISpot), an intracellular cytokine staining (ICS) assay, or an activation induced marker assay (AIM). Each of these assays is commonly used to detect cell based (e.g., T cell) immune responses to vaccines and well known to the person of ordinary skill in the art. See, e.g., Bowyer, Georgina et al. “Activation-induced Markers Detect Vaccine-Specific CD4⁺ T Cell Responses Not Measured by Assays Conventionally Used in Clinical Trials.” Vaccines vol. 6, 3 50. 31 Jul. 2018, doi:10.3390/vaccines6030050, the entire contents of which is incorporated by reference herein for all purposes.

5.13.3 Methods of Preventing, Ameliorating, and/or Treating Infection

Provided herein are methods of preventing, ameliorating, and/or treating a SARS-CoV-2 infection in a subject in need thereof, the method comprising administering to the subject (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing), to thereby prevent, ameliorate, and/or treat the SARS-CoV-2 infection in the subject.

In some embodiments, the method comprises preventing a SARS-CoV-2 infection in the subject in need thereof. In some embodiments, the method comprises ameliorating a SARS-CoV-2 infection in the subject in need thereof. In some embodiments, the method comprises treating a SARS-CoV-2 infection in the subject in need thereof.

In some embodiments, the SARS-CoV-2 spike protein or polypeptide (e.g., the SARS-CoV-2 protein or peptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to prevent, ameliorate, and/or treat the SARS-CoV-2 infection the subject. In some embodiments, the SARS-CoV-2 spike protein or polypeptide (e.g., the SARS-CoV-2 protein or peptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to prevent the SARS-CoV-2 infection the subject. In some embodiments, the SARS-CoV-2 spike protein or polypeptide (e.g., the SARS-CoV-2 protein or peptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to ameliorate the SARS-CoV-2 infection the subject. In some embodiments, the SARS-CoV-2 spike protein or polypeptide (e.g., the SARS-CoV-2 protein or peptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to treat the SARS-CoV-2 infection the subject.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, or the pharmaceutical composition is administered to the subject as a prophylactic treatment. In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, or the pharmaceutical composition is administered as a treatment after the onset of at least one symptom of a SARS-CoV-2 infection or a SARS-CoV-2 infection associated disease.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject after a determination that the subject does or does not have a SARS-CoV-2 infection.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition prevents infection with SARS-CoV-2, reduces the likelihood of infection with SARS-CoV-2, reduces the likelihood of developing an established infection after challenge with SARS-CoV-2, reduces the duration of a SARS-CoV-2 infection, prevents or delays onset one or more symptoms of COVID-19, reduces the frequency and/or severity one or more symptoms of COVID-19, and/or reduces the risk of hospitalization or death associated with COVID-19, or any combination of thereof.

Exemplary COVID-19 symptoms include, but are not limited to, shortness of breath, difficulty breathing, respiratory rate greater than or equal to 20 breaths per minutes, abnormal SpO2, clinical or radiological evidence of lower respiratory tract disease, radiological evidence of deep vein thrombosis, respiratory failure, evidence of shock, significant renal, hepatic, and neurological dysfunction.

The SARS-CoV-2 spike proteins (e.g., immunogens (or immunogenic fragments or immunogenic variants thereof)), the nucleic acid molecules (e.g., RNA molecules, e.g., mRNA molecules), the vectors, the carriers, the vaccine compositions, and the pharmaceutical compositions described herein may be administered as a prime and/or a boost in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a prime in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a boost in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a vaccine prime and a vaccine boost in a homologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a prime in a heterologous prime-boost regimen. The boost vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, the SARS-CoV-2 spike proteins (e.g., immunogens (or immunogenic fragments or immunogenic variants thereof)), the nucleic acid molecules (e.g., RNA molecules, e.g., mRNA molecules), the vectors, the carriers, the vaccine compositions, and the pharmaceutical compositions described herein are administered to a subject as a boost in a heterologous prime-boost regimen. The prime vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, a single dose of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition is administered to the subject. In some embodiments, a series of doses of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition are administered to the subject (e.g., two doses given at a set interval (e.g., 2 weeks, 3 weeks apart) or within a range (e.g., 2-6 weeks apart)).

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition is administered to the subject in a therapeutically effective amount. In some embodiments, wherein an mRNA molecule encoding a SARS-CoV-2 spike protein (e.g., a SARS-CoV-2 spike protein or polypeptide immunogen (or immunogenic fragment and/or immunogenic variant thereof)) described herein (e.g., a vaccine or pharmaceutical composition comprising the same) is administered to the subject, the mRNA molecule is administered at a dose from about 30-200 mcg (e.g., 30 mcg, 50 mcg, 75 mcg, 100 mcg, 150 mcg, or 200 mcg).

Further provided herein are (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing) for use in the manufacture of a medicament for the prevention, amelioration, or treatment of a SARS-CoV-2 infection in a subject in need thereof.

Further provided herein are (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing) for use in a method of preventing, ameliorating, and/or treating a SARS-CoV-2 infection in a subject in need thereof, the method comprising administering the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition to the subject in need thereof.

### 5.13.4 Methods of Vaccination Against SARS-CoV-2

Provided herein are, inter alia, various methods of vaccinating subjects (e.g., human subjects) against SARS-CoV-2. As such, provided below in §§ 5.13.4.1 and 5.13.4.2, are various methods of vaccinating subjects utilizing one or more of the SARS-CoV-2 spike proteins (e.g., immunogens (or immunogenic fragments or immunogenic variants thereof)), the nucleic acid molecules (e.g., RNA molecules, e.g., mRNA molecules), the vectors, the carriers, the vaccine compositions, and the pharmaceutical compositions described herein.

### 5.13.4.1 Methods of Vaccinating a Subject Against SARS-CoV-2

Provided herein are methods of vaccinating a subject against SARS-CoV-2, the method comprising administering to the subject to the subject (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing), to thereby vaccinate the subject against SARS-CoV-2.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to vaccinate the subject against SARS-CoV-2.

In some embodiments, the SARS-CoV-2 spike proteins (e.g., immunogens (or immunogenic fragments or immunogenic variants thereof)), the nucleic acid molecules (e.g., RNA molecules, e.g., mRNA molecules), the vectors, the carriers, the vaccine compositions, and the pharmaceutical compositions described herein are administered as a prime and/or a boost in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a prime in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a boost in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a vaccine prime and a vaccine boost in a homologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a prime in a heterologous prime-boost regimen. The boost vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, the SARS-CoV-2 spike proteins (e.g., immunogens (or immunogenic fragments or immunogenic variants thereof)), the nucleic acid molecules (e.g., RNA molecules, e.g., mRNA molecules), the vectors, the carriers, the vaccine compositions, and the pharmaceutical compositions described herein are administered to a subject as a boost in a heterologous prime-boost regimen. The prime vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, a single dose of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition is administered to the subject. In some embodiments, a series of doses of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition are administered to the subject (e.g., two doses given at a set interval (e.g., 2 weeks, 3 weeks apart) or within a range (e.g., 2-6 weeks apart)).

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition is administered to the subject in a therapeutically effective amount. In some embodiments, wherein an mRNA molecule encoding a SARS-CoV-2 spike protein (e.g., a SARS-CoV-2 spike protein or polypeptide immunogen (or immunogenic fragment and/or immunogenic variant thereof)) described herein (e.g., a vaccine or pharmaceutical composition comprising the same) is administered to the subject, the mRNA molecule is administered at a dose from about 30-200 mcg (e.g., 30 mcg, 50 mcg, 75 mcg, 100 mcg, 150 mcg, or 200 mcg).

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, or the pharmaceutical composition is administered to the subject as a prophylactic treatment. In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, or the pharmaceutical composition is administered as a treatment after the onset of at least one symptom of a SARS-CoV-2 infection or a SARS-CoV-2 infection associated disease.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject after a determination that the subject does or does not have a SARS-CoV-2 infection.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition prevents infection with SARS-CoV-2, reduces the likelihood of infection with SARS-CoV-2, reduces the likelihood of developing an established infection after challenge with SARS-CoV-2, reduces the duration of a SARS-CoV-2 infection, prevents or delays onset one or more symptoms of COVID-19, reduces the frequency and/or severity one or more symptoms of COVID-19, and/or reduces the risk of hospitalization or death associated with COVID-19, or any combination of thereof.

Exemplary COVID-19 symptoms include, but are not limited to, shortness of breath, difficulty breathing, respiratory rate greater than or equal to 20 breaths per minutes, abnormal SpO2, clinical or radiological evidence of lower respiratory tract disease, radiological evidence of deep vein thrombosis, respiratory failure, evidence of shock, significant renal, hepatic, and neurological dysfunction.

Further provided herein are (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing) for use in the manufacture of a medicament for the vaccination a subject against SARS-CoV-2.

Further provided herein are (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing) for use in a method of vaccinating a subject against SARS-CoV-2, the method comprising administering the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition to the subject in need thereof.

5.13.4.2 Methods of Vaccinating a Subject Utilizing a SARS-CoV-2 mRNA Vaccine

Provided herein are methods of vaccinating in a subject against SARS-CoV-2, the method comprising administering to the subject to the subject (a) an mRNA molecule (e.g., an mRNA molecule described herein) encoding a SARS-CoV-2 spike protein (e.g., a SARS-CoV-2 spike protein or polypeptide immunogen (or immunogenic fragment and/or immunogenic variant thereof)) described herein (or a fusion or conjugate thereof), (b) a vector comprising the mRNA molecule, (c) a carrier comprising the mRNA molecule or the vector, (d) a vaccine composition comprising the mRNA molecule, the vector, or the carrier, or (e) a pharmaceutical composition comprising the mRNA molecule, the vector, the carrier, or the vaccine composition, to thereby vaccinate the subject against SARS-CoV-2.

In some embodiments, the mRNA molecule is formulated in a lipid nanoparticle, the vaccine composition having the following characteristics: (a) the LNPs comprise a cationic lipid, a neutral lipid, a cholesterol, and a PEG lipid, (b) the LNPs have a mean particle size of between 80 nm and 160 nm, and (c) the mRNA comprises: (i) a 5′-cap structure; (ii) a 5′-UTR; (iii) N1-methyl-pseudouridine, cytosine, adenine, and guanine; (iv) a 3′-UTR; and (v) a poly-A region

In some embodiments, the SARS-CoV-2 spike protein or polypeptide (e.g., a SARS-CoV-2 spike protein or polypeptide immunogen (or immunogenic fragment and/or immunogenic variant thereof)), the mRNA molecule, the vector, the carrier, the vaccine composition, or the pharmaceutical composition is administered to the subject in an amount and for a time sufficient to vaccinate the subject against SARS-CoV-2.

In some embodiments, the mRNA molecules, the vectors, the carriers, the vaccine compositions, and the pharmaceutical compositions described herein are administered as a prime and/or a boost in a homologous or heterologous prime-boost regimen.

In some embodiments, the mRNA molecule, the vector, the carrier, the vaccine composition, or the pharmaceutical composition described herein is administered to the subject as a prime in a homologous or heterologous prime-boost regimen.

In some embodiments, the mRNA molecule, the vector, the carrier, the vaccine composition, or the pharmaceutical composition described herein is administered to the subject as a boost in a homologous or heterologous prime-boost regimen.

In some embodiments, the mRNA molecule, the vector, the carrier, the vaccine composition, or the pharmaceutical composition described herein is administered to the subject as a vaccine prime and a vaccine boost in a homologous prime-boost regimen.

In some embodiments, the mRNA molecule, the vector, the carrier, the vaccine composition, or the pharmaceutical composition described herein is administered to the subject as a prime in a heterologous prime-boost regimen. The boost vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, the mRNA molecules, the vectors, the carriers, the vaccine compositions, and the pharmaceutical compositions described herein are administered to a subject as a boost in a heterologous prime-boost regimen. The prime vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, a single dose of the mRNA molecule, the vector, the carrier, the vaccine composition, or the pharmaceutical composition is administered to the subject. In some embodiments, a series of doses of the mRNA molecule, the vector, the carrier, the vaccine composition, or the pharmaceutical composition are administered to the subject (e.g., two doses given at a set interval (e.g., 2 weeks, 3 weeks apart) or within a range (e.g., 2-6 weeks apart)).

In some embodiments, the mRNA molecule, the vector, the carrier, the vaccine composition, or the pharmaceutical composition is administered to the subject in a therapeutically effective amount. In some embodiments, the mRNA molecule is administered at a dose from about 30-200 mcg (e.g., 30 mcg, 50 mcg, 75 mcg, 100 mcg, 150 mcg, or 200 mcg).

In some embodiments, the mRNA molecule, the vector, the carrier, or the pharmaceutical composition is administered to the subject as a prophylactic treatment. In some embodiments, the mRNA molecule, the vector, the carrier, or the pharmaceutical composition is administered as a treatment after the onset of at least one symptom of a SARS-CoV-2 infection or a SARS-CoV-2 infection associated disease.

In some embodiments, the mRNA molecule, the vector, the carrier, the vaccine composition, or the pharmaceutical composition is administered to the subject after a determination that the subject does or does not have a SARS-CoV-2 infection.

In some embodiments, the mRNA molecule, the vector, the carrier, the vaccine composition, or the pharmaceutical composition prevents infection with SARS-CoV-2, reduces the likelihood of infection with SARS-CoV-2, reduces the likelihood of developing an established infection after challenge with SARS-CoV-2, reduces the duration of a SARS-CoV-2 infection, prevents or delays onset one or more symptoms of COVID-19, reduces the frequency and/or severity one or more symptoms of COVID-19, and/or reduces the risk of hospitalization or death associated with COVID-19, or any combination of thereof.

Exemplary COVID-19 symptoms include, but are not limited to, shortness of breath, difficulty breathing, respiratory rate greater than or equal to 20 breaths per minutes, abnormal SpO2, clinical or radiological evidence of lower respiratory tract disease, radiological evidence of deep vein thrombosis, respiratory failure, evidence of shock, significant renal, hepatic, and neurological dysfunction.

Further provided herein are (a) an mRNA molecule (e.g., an mRNA molecule described herein) encoding a SARS-CoV-2 spike protein (e.g., a SARS-CoV-2 spike protein or polypeptide immunogen (or immunogenic fragment and/or immunogenic variant thereof)) described herein (or a fusion or conjugate thereof), (b) a vector comprising the mRNA molecule, (c) a carrier comprising the mRNA molecule or the vector, (d) a vaccine composition comprising the mRNA molecule, the vector, or the carrier, or (e) a pharmaceutical composition comprising the mRNA molecule, the vector, the carrier, or the vaccine composition for use in the manufacture of a medicament for the vaccination a subject against SARS-CoV-2.

Further provided herein are (a) an mRNA molecule (e.g., an mRNA molecule described herein) encoding a SARS-CoV-2 spike protein (e.g., a SARS-CoV-2 spike protein or polypeptide immunogen (or immunogenic fragment and/or immunogenic variant thereof)) described herein (or a fusion or conjugate thereof), (b) a vector comprising the mRNA molecule, (c) a carrier comprising the mRNA molecule or the vector, (d) a vaccine composition comprising the mRNA molecule, the vector, or the carrier, or (e) a pharmaceutical composition comprising the mRNA molecule, the vector, the carrier, or the vaccine composition for use in the vaccination a subject against SARS-CoV-2. In some embodiments, the method comprises administering the mRNA molecule, the vector, the carrier, the vaccine composition, or the pharmaceutical composition to the subject in need thereof.

### 5.14 Kits

Provided herein are kits comprising at least one SARS-CoV-2 spike protein or polypeptide (e.g., a SARS-CoV-2 spike protein or polypeptide immunogen (or immunogenic fragment and/or immunogenic variant thereof)) described herein, a nucleic acid molecule (e.g., RNA molecule, e.g., mRNA molecule) described herein, a vector described herein, a carrier described herein, a vaccine composition described herein, and/or a pharmaceutical composition described herein. In addition, the kit may comprise a liquid vehicle for solubilizing or diluting any one of the foregoing, and/or technical instructions. The technical instructions of the kit may contain information about administration and dosage and subjects (e.g., subject groups).

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is provided in a separate part of the kit, wherein the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is optionally lyophilized, spray-dried, or spray-freeze dried. The kit may further contain as a part a vehicle (e.g., buffer solution) for solubilizing the dried or lyophilized pharmaceutical composition.

In some embodiments, the kit comprises a single dose container of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition. In some embodiments, the kit comprises a multi-dose container for administration of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein and/or an administration device (e.g., an injector for intradermal injection or a syringe for intramuscular injection).

In some embodiments, the kit comprises an adjuvant in a separate container from the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein. The kit may further contain technical instructions for mixing the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein and the adjuvant prior to administration or for co-administration.

Any of the kits described herein may be used in any of the methods described herein, e.g., in § 5.13. Any of the kits described herein may be used in a treatment or prophylaxis as defined herein (e.g., for the treatment, amelioration, and/or prophylaxis of SARS-CoV-2 infection).

### 6. EXAMPLES

### 6.1 Example 1. SARS-CoV-2 RNA Vaccines

The following example provides an exemplary method of preparing an mRNA vaccine comprising an mRNA encoding any one or a plurality of the immunogens identified herein (e.g., an immunogen comprising an amino acid substitution set forth in Table 2).

### 6.1.1 DNA Preparation

DNA constructs comprising SARS-CoV-2 proteins (e.g., immunogens) comprising at least one of the amino acid substitutions set forth in Table 2 are prepared and used for subsequent RNA in vitro transcription. Preparation of the DNA coding sequences can include codon optimization for stabilization and expression by introducing specific codons to generate a DNA coding sequence with an optimized G/C content (as discussed herein). Optimized coding sequences are introduced into a DNA plasmid comprising a 3′-UTR, a 5-UTR, and polyadenylation sequence. The obtained DNA plasmids are transformed and propagated in bacteria using common protocols known in the art. The DNA plasmids are subsequently extracted, purified, and used for RNA in vitro transcription.

### 6.1.2 RNA In Vitro Transcription

The DNA plasmids are enzymatically linearized using a restriction enzyme used for DNA dependent RNA in vitro transcription using T7 RNA polymerase in the presence of a nucleotide mixture (ATP/GTP/CTP/UTP) and a 5′ cap (or analog) under suitable buffer conditions. The obtained RNA constructs are purified using a suitable method known in the art e.g., RP-HPLC. The RNA in vitro transcription is performed in the presence of modified nucleotides for incorporation in the RNA e.g., pseudouridine or N1-methylpseudouridine (mlψ) instead of UTP. In some instances, the 5′ cap is enzymatically added to the RNA after in vitro transcription using capping enzymes as commonly known in the art.

### 6.1.3 Preparation of LNPs and Encapsulated RNAs

LNPs are prepared using according to the general procedures known in the art using e.g., cationic lipids, structural lipids, a PEG-lipids, and cholesterol see, e.g., WO2015199952, WO2017004143 and WO2017075531, the full contents of each of which is incorporated by reference herein for al purposes. The lipid solution (in ethanol) is mixed with RNA (in aqueous solution) in a small T-piece to encapsulate the mRNA in the LNPs, see, e.g., Reichmuth, Andreas M et al. “mRNA vaccine delivery using lipid nanoparticles.” Therapeutic delivery vol. 7, 5 (2016): 319-34. doi:10.4155/tde-2016-0006, the entire contents of which is incorporated by reference herein for all purposes. The LNP formulated mRNA is rebuffered as needed via dialysis and concentrated to a target concentration using ultracentrifugation.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. A nucleic acid molecule comprising at least one coding region encoding a SARS-CoV-2 spike protein,
wherein the amino acid sequence of the encoded SARS-CoV-2 spike protein comprises at least one set of amino acid substitutions set forth in Table 2 or Table 4.

2.-82. (canceled)

83. A vector comprising the nucleic acid molecule of claim 1.

84. (canceled)

85. A conjugate comprising the nucleic acid molecule of claim 1 operably connected to a heterologous moiety.

86. A composition comprising the nucleic acid molecule of claim 1.

87.-93. (canceled)

94. The composition of claim 86, further comprising an adjuvant.

95.-98. (canceled)

99. A SARS-CoV-2 spike protein comprising an amino acid sequence that comprises at least one set of amino acid substitutions set forth in Table 2 or Table 4.

100.-171. (canceled)

172. A fusion protein comprising the SARS-CoV-2 spike protein of claim 99 operably connected to a heterologous protein.

173. A conjugate comprising the SARS-CoV-2 spike protein of claim 99 operably connected to a heterologous moiety.

174. A composition comprising the SARS-CoV-2 spike protein of claim 99.

175.-186. (canceled)

187. A carrier comprising the nucleic acid molecule of claim 1.

188.-191. (canceled)

192. A vaccine composition comprising the nucleic acid molecule of claim 1.

193.-198. (canceled)

199. A pharmaceutical composition comprising the nucleic acid molecule of claim 1, and a pharmaceutically acceptable excipient.

200.-201. (canceled)

202. A host cell comprising the nucleic acid molecule of claim 1.

203. A kit comprising the nucleic acid molecule of claim 1.

204. (canceled)

205. A method of delivering a nucleic acid molecule to a subject in need thereof, the method comprising administering to the subject the nucleic acid molecule of claim 1, to thereby deliver the nucleic acid molecule to the subject.

206. A method of inducing or enhancing an immune response in a subject in need thereof, the method comprising administering to the subject the nucleic acid molecule of claim 1, to thereby induce or enhance an immune response the subject.

207. A method of preventing, ameliorating, or treating a SARS-CoV-2 infection in a subject in need thereof, the method comprising administering to the subject the nucleic acid molecule of claim 1, to thereby prevent, ameliorate, or treat the SARS-CoV-2 infection the subject.

208. A method of vaccinating a subject against SARS-CoV-2, the method comprising administering to the subject the nucleic acid molecule of claim 1, to thereby vaccinate the subject against SARS-CoV-2.

209. A method of vaccinating a subject against SARS-CoV-2, the method comprising administering to the subject (a) an mRNA molecule encoding the SARS-CoV-2 spike protein of claim 99, (b) a vector comprising the mRNA molecule, (c) a carrier comprising the mRNA molecule or the vector, (d) a vaccine composition comprising the mRNA molecule, the vector, or the carrier, or (e) a pharmaceutical composition comprising the mRNA molecule, the vector, the carrier, or the vaccine composition, to thereby vaccinate the subject against SARS-CoV-2, to thereby vaccinate the subject against SARS-CoV-2.

210. A method of vaccinating a subject against SARS-CoV-2, the method comprising administering to the subject the vaccine composition of claim 192, to thereby vaccinate the subject against SARS-CoV-2.

211.-220. (canceled)
